SEQUENCE LISTING (678 sequences)

SEQ ID NO: 1 - protein, 25 aa, Aquifex aeolicus
SEEEFYTALSETSIFGGSKEKAVVI

SEQ ID NO: 2 - protein, 25 aa, Thermotoga maritima
KIDFIRSLLRTKTIFSNKTIIDIVN

SEQ ID NO: 3 - protein, 22 aa, Chloroflexus aurantiacus
QLVAACEAHPFLAERRLVIVYD

SEQ ID NO: 4 - protein, 25 aa, Deinococcus radiodurans
VSAETLGPHLAPSLFGDGGVVVDFE

SEQ ID NO: 5 - protein, 25 aa, Porphyromonas gingivalis
SVADIANEARRFPMMGRRQLIVVRE

SEQ ID NO: 6 - protein, 25 aa, Bacteroides fragilis
DVATVINAAKRYPMMSEHQVVIVKE

SEQ ID NO: 7 - protein, 25 aa, Cytophaga hutchinsonii
NVSTILQNARKYPMFSERQVVMVKE

SEQ ID NO: 8 - protein, 25 aa, Chlorobium tepidum
TLGQIVSAASEYPMFTEKKLVVVRQ

SEQ ID NO: 9 - protein, 25 aa, Chlamydia trachomatis
LQQELLSWTDHFGLFASQETIGIYQ

SEQ ID NO: 10 - protein, 25 aa, Chlamydophila pneumoniae
MPATLMSWTETFALFQEHETLGIIH

SEQ ID NO: 11 - protein, 25 aa, Nostoc punctiforme
AAIQALNQVMTPTFGAGGRLVWLIN

SEQ ID NO: 12 - protein, 25 aa, Anabaena sp.
AAIQALNQVMTPAFGAGGRLVWLMN

SEQ ID NO: 13 - protein, 25 aa, Synechocystis sp.
ATQRGLEQALTPPFGSGDRLVWVVD

SEQ ID NO: 14 - protein, 25 aa, Prochlorococcus marinus
QIKQAFDEILTPPLGDGSRVVVLKN

SEQ ID NO: 15 - protein, 25 aa, Prochlorococcus marinus
QASQALAEARTPPFGSGGRLVLLQR

SEQ ID NO: 16 - protein, 25 aa, Synechococcus sp.
QAAQALDEARTPPFASGERLVLLQR

SEQ ID NO: 17 - protein, 25 aa, Treponema denticola
GMGDVISLLQNASLFSSAKLIILKS

SEQ ID NO: 18 - protein, 25 aa, Treponema pallidum
PVADLVDLLRTRALFADAVCVVLYN

SEQ ID NO: 19 - protein, 25 aa, Borrelia burgdorferi
SAVGFAEKLFSNSFFSKKEIFIVYE

SEQ ID NO: 20 - protein, 25 aa, Magnetospirillum magnetotacticum
IPSRLADEAAAMALGGGRRVVVLRD

SEQ ID NO: 21 - protein, 25 aa, Magnetospirillum magnetotacticum
DPGRLVDEAGTVGLFGGSRTIWVRS

SEQ ID NO: 22 - protein, 25 aa, Rhodopseudomonas palustris
EPSRLVDEALAIPMFGGRRAIRVRA

SEQ ID NO: 23 - protein, 25 aa, Mesorhizobium loti
DEGRLLDEARTVPMFSDRRLLWVRN

SEQ ID NO: 24 - protein, 25 aa, Brucella suis
DPAKLADEAGTISMFGGQRLIWIKN

SEQ ID NO: 25 - protein, 25 aa, Sinorhizobium meliloti
GAGSVLDEVNAIGLFGGDKLVWVRG

SEQ ID NO: 26 - protein, 25 aa, Agrobacterium tumefaciens
DPGRLLDEVNAIGLFGGEKLVWVKS

SEQ ID NO: 27 - protein, 25 aa, Caulobacter crescentus
DPAKLEDELSAMSLMGGRRLVRLRL

SEQ ID NO: 28 - protein, 25 aa, Rhodobacter sphaeroides
DPAALMDAMTAKGFFEGPRAVLVEE

SEQ ID NO: 29 - protein, 25 aa, Rickettsia conorii
NISSLEILLNSSNFFGQKELIKIRS

SEQ ID NO: 30 - protein, 25 aa, Rickettsia prowazekii
NILSLDILLNSPNFFGQKELIKVRS

SEQ ID NO: 31 - protein, 25 aa, Wolbachia sp.
SPSLLFSELANVSMFTSKKLIKLIN

SEQ ID NO: 32 - protein, 25 aa, Neisseria gonorrhoeae
DWNELLQTAGNAGLFADLKLLELHI

SEQ ID NO: 33 - protein, 25 aa, Neisseria meningitidis
DWNELLQTAGSAGLFADLKLLELHI

SEQ ID NO: 34 - protein, 25 aa, Nitrosomonas europaea
DWMNLFQWGRQSSLFSERRMLDLRI

SEQ ID NO: 35 - protein, 25 aa, Bordetella pertussis
DWSAVAAATQSVSLFGDRRLLELKI

SEQ ID NO: 36 - protein, 25 aa, Burkholderia pseudomallei
DWSTLIGASQAMSLFGERQLVELRI

SEQ ID NO: 37 - protein, 25 aa, Burkholderia cepacia
DWSSLLGASQSMSLFGDRQLVELRI

SEQ ID NO: 38 - protein, 25 aa, Burkholderia mallei
DWSTLIGASQAMSLFGERQLVELRI

SEQ ID NO: 39 - protein, 25 aa, Ralstonia metallidurans
QWGQVIEAQQSMSLFGDRKIVELRI

SEQ ID NO: 40 - protein, 25 aa, Acidothiobacillus ferrooxidans
IWDALRDERDAGSLFAAQRVLLLRL

SEQ ID NO: 41 - protein, 25 aa, Xylella fastidiosa
DWQQLASSFNAPSLFSSRRLIEIRL

SEQ ID NO: 42 - protein, 25 aa, Legionella pneumophila
EWHVVLEETNNYSLFYQTVILTIFF

SEQ ID NO: 43 - protein, 25 aa, Coxiella burnetii
HWQSLTQSFDNFSLLSDKTLIELRN

SEQ ID NO: 44 - protein, 25 aa, Methylococcus capsulatus
SWSTFLEAGDSVPLFGDRRILDLRL

SEQ ID NO: 45 - protein, 25 aa, Pseudomonas aeruginosa
DWGLLLEAGASLSLFAEKRLIELRL

SEQ ID NO: 46 - protein, 25 aa, Pseudomonas putida
DWGTLLQAGASLSLFAQRRLLELRL

SEQ ID NO: 47 - protein, 25 aa, Pseudomonas syringae
DWGTLLQAGASMSLFAERRLLELRL

SEQ ID NO: 48 - protein, 25 aa, Pseudomonas fluorescens
DWGTLLQAGASMSLFAEKRLLELRL

SEQ ID NO: 49 - protein, 25 aa, Shewanella putrefaciens
NWGDLTQEWQAMSLFSSRRIIELTL

SEQ ID NO: 50 - protein, 25 aa, Vibrio cholerae
DWNAVYDCCQALSLFSSRQLIEIEI

SEQ ID NO: 51 - protein, 25 aa, Pasteurella multocida
NWSDLFERCQSIGLFFNKQILFLNL

SEQ ID NO: 52 - protein, 25 aa, Haemophilus influenzae
DWAQLIESCQSIGLFFSKQILSLNL

SEQ ID NO: 53 - protein, 25 aa, Haemophilus ducreyi
KWEQLFESVQNFGLFFSRQIIILNL

SEQ ID NO: 54 - protein, 25 aa, Actinobacillus actinomycetemcomitans
DWNDLFERVQSMGLFFNKQLIILDL

SEQ ID NO: 55 - protein, 25 aa, Buchnera sp.
DWKKIILFYKTNNLFFKKTTLVINF

SEQ ID NO: 56 - protein, 25 aa, Escherichia coli
DWNAIFSLCQAMSLFASRQTLLLLL

SEQ ID NO: 57 - protein, 25 aa, Salmonella typhi
DWGSLFSLCQAMSLFASRQTLVLQL

SEQ ID NO: 58 - protein, 25 aa, Salmonella typhimurium
DWGSLFSLCQAMSLFASRQTLVLQL

SEQ ID NO: 59 - protein, 25 aa, Klebsiella pneumoniae
PTGRRFSLKPGDELFASRQTLLLIL

SEQ ID NO: 60 - protein, 25 aa, Yersinia pestis
EWEHIFSLCQALSLFASRQTLLLSF

SEQ ID NO: 61 - protein, 25 aa, Yersinia pseudotuberculosis
EWEHIFSLCQALSLFASRQTLLLSF

SEQ ID NO: 62 - protein, 25 aa, Desulfovibrio vulgaris
LPPVFWEHLTLQGLFGSPRALVVRN

SEQ ID NO: 63 - protein, 25 aa, Geobacter sulfurreducens
KGDDIATAAQTLPMFADRRMVLVKR

SEQ ID NO: 64 - protein, 25 aa, Helicobacter pylori
EKSQIATLLEQDSLFGGSSLVILKL

SEQ ID NO: 65 - protein, 25 aa, Campylobacter jejuni
NFTRASDFLSAGSLFSEKKLLEIKT

SEQ ID NO: 66 - protein, 25 aa, Streptomyces coelicolor
LQPGTLAELTSPSLFAERKVVVVRN

SEQ ID NO: 67 - protein, 25 aa, Thermobifida fusca
VSAGKLVEVTSPSLFGDRRVVVLRS

SEQ ID NO: 68 - protein, 25 aa, Mycobacterium avium
VSTYELAELLSPSLFAEERIVVLEA

SEQ ID NO: 69 - protein, 25 aa, Mycobacterium leprae
VGTYELTELLSPSLFADERIVVLEA

SEQ ID NO: 70 - protein, 25 aa, Mycobacterium smegmatis
VSTSELAELLSPSLFAEERLVVLEA

SEQ ID NO: 71 - protein, 25 aa, Mycobacterium tuberculosis
VGAYELAELLSPSLFAEERIVVLGA

SEQ ID NO: 72 - protein, 25 aa, Corynebacterium diptheriae
VNASELIQLTSPSLFGEDRIIVLTN

SEQ ID NO: 73 - protein, 25 aa, Dehalococcoides ethenogenes
TAAELQNYVQTIPFLAPARLVMVNG

SEQ ID NO: 74 - protein, 20 aa, Clostridium difficile
VLNHLISSIETLPFMDDRKI

SEQ ID NO: 75 - protein, 25 aa, Carboxydothermus hydrogenoformans
LPEEVVARAETVSFFGQRFIVVKNC

SEQ ID NO: 76 - protein, 25 aa, Bacillus halodurans
PIEAALEEAETVPFFGSKRVVILKD

SEQ ID NO: 77 - protein, 25 aa, Bacillus stearothermophilus
PIEAALEEAETVPFFGERRVILIKH

SEQ ID NO: 78 - protein, 25 aa, Bacillus subtilis
PLDQAIADAETFPFMGERRLVIVKN

SEQ ID NO: 79 - protein, 25 aa, Staphylococcus aureus
EIAPIVEETLTLPFFSDKKAILVKN

SEQ ID NO: 80 - protein, 25 aa, Staphylococcus epidermidis
DLTPIIEETLTMPFFSNKKAIVVKN

SEQ ID NO: 81 - protein, 25 aa, Bacillus anthracis
YLEDVVEDARTLPFFGERKVLLIKS

SEQ ID NO: 82 - protein, 25 aa, Listeria innocua
PIEVVIQEAESMPFFGDKRLVMANN

SEQ ID NO: 83 - protein, 25 aa, Listeria monocytogenes
PIEVVIQEAESMPFFGDKRLVMANN

SEQ ID NO: 84 - protein, 25 aa, Listeria monocytogenes
PIEVVVQEAESMPFFGDKRLVMANN

SEQ ID NO: 85 - protein, 25 aa, Enterococcus faecalis
PLSAAIAEAETIPFFGDYRLVFVEN

SEQ ID NO: 86 - protein, 25 aa, Enterococcus faecium
SLDEVVAEAETLPFFGDQRLVFVEN

SEQ ID NO: 87 - protein, 25 aa, Lactococcus lactis
NSDLALEDLESLPFFSDSRLVILEN

SEQ ID NO: 88 - protein, 25 aa, Streptococcus equi
LYQTAEMDLVSMPFFADQKVVIFDH

SEQ ID NO: 89 - protein, 25 aa, Streptococcus agalactiae
DYQNAELDLESLPFLSDYKVVIFDQ

SEQ ID NO: 90 - protein, 25 aa, Streptococcus pyogenes
AYQDAEMDLVSLPFFAEQKVVIFDH

SEQ ID NO: 91 - protein, 25 aa, Streptococcus mutans
SYQDAEMDLESLPFFADEKIVIFDN

SEQ ID NO: 92 - protein, 25 aa, Streptococcus gordonii
DYQQVELDLVSLPFFSDEKIIILDH

SEQ ID NO: 93 - protein, 25 aa, Streptococcus pneumoniae
VYKDVELELVSLPFFADEKIVILDY

SEQ ID NO: 94 - protein, 25 aa, Ureaplasma urealyticum
SLISFKNLIEQDDLFNSNKIYLFKN

SEQ ID NO: 95 - protein, 25 aa, Mycoplasma genitalium
KDLKQLYDLFSQPLFGSNNEKFIVN

SEQ ID NO: 96 - protein, 25 aa, Mycoplasma pneumoniae
DVNKLYDVVLNQNLFAEDTKPILIH

SEQ ID NO: 97 - protein, 25 aa, Mycoplasma pulmonis
EIDDLLNDIVQKDLFSPNKIIHIKN

SEQ ID NO: 98 - protein, 25 aa, Clostridium acetobutylicum
EFEDILNACETVPFMSEKRMVVVYR

SEQ ID NO: 99 - protein, 15 aa, Magnetococcus sp.
SSQTATTQPQQLSLF

SEQ ID NO: 100 - protein, 25 aa, Cytophaga hutchinsonii
KLSNLVHGNYQISLFEDSEKNQNLY

SEQ ID NO: 101 - protein, 25 aa, Treponema denticola
MNIESDIPEAQTELFYSEKNVKKRK

SEQ ID NO: 102 - protein, 20 aa, Magnetospirillum magnetotacticum
TDLCPAEDADPPDLFGPRPA

SEQ ID NO: 103 - protein, 25 aa, Magnetospirillum magnetotacticum
LGELSRTERRQLDLLTNDEPVRKRL

SEQ ID NO: 104 - protein, 25 aa, Methylobacterium extorquens
GDLCGAIHADRGDLADQGIERVARR

SEQ ID NO: 105 - protein, 25 aa, Rhodopseudomonas palustris
SALTEQTGPAEDDMLDRRSAHAERA

SEQ ID NO: 106 - protein, 16 aa, Mesorhizobium loti
LGDVLPPDQRQLRFEL

SEQ ID NO: 107 - protein, 25 aa, Mesorhizobium loti
SDLSDDDKADPPDLVDVQSRKRAMA

SEQ ID NO: 108 - protein, 26 aa, Mesorhizobium loti
VSHLEESAELQLDLPLGLADEKRRPG

SEQ ID NO: 109 - protein, 25 aa, Brucella suis
SDLSPSDRADPPDLVDIQATKRAVA

SEQ ID NO: 110 - protein, 25 aa, Sinorhizobium meliloti
SDLVDPDLADPPDLVDPQASRRAAA

SEQ ID NO: 111 - protein, 16 aa, Sinorhizobium meliloti
LDTVDDRSEPQLALAL

SEQ ID NO: 112 - protein, 25 aa, Agrobacterium tumefaciens
SDLRDAGLADPPDLVDRQATRRAAA

SEQ ID NO: 113 - protein, 16 aa, Agrobacterium tumefaciens
DQEAEDEEQPQLDLAL

SEQ ID NO: 114 - protein, 25 aa, Caulobacter crescentus
LTEFVDADTAGADMFADEERRALKS

SEQ ID NO: 115 - protein, 25 aa, Rhodobacter sphaeroides
AGAAEADLTGTGDLLDPNAGRRIAA

SEQ ID NO: 116 - protein, 25 aa, Rhodobacter capsulatus
DLSPAGGRDPIGDLLDPQATARAAA

SEQ ID NO: 117 - protein, 16 aa, Sphingomonas aromaticivorans
AEDGPSGAALQAELPF

SEQ ID NO: 118 - protein, 16 aa, Neisseria gonorrhoeae
GVGRLVPKNQQQDLWA

SEQ ID NO: 119 - protein, 16 aa, Neisseria meningitidis
GVGHLVPKNQQQDLWA

SEQ ID NO: 120 - protein, 15 aa, Nitrosomonas europaea
SALLKENYYFQEELF

SEQ ID NO: 121 - protein, 19 aa, Bordetella pertussis
FPDAQAEAPRQAELFGDAF

SEQ ID NO: 122 - protein, 15 aa, Burkholderia pseudomallei
IDEDTAERHGQIALF

SEQ ID NO: 123 - protein, 20 aa, Burkholderia cepacia
ALTPPRRLPVQADLPFASDE

SEQ ID NO: 124 - protein, 25 aa, Burkholderia mallei
IDEDTAERHGQIALFDDEDMSDEDA

SEQ ID NO: 125 - protein, 26 aa, Ralstonia metallidurans
ADQGDDPAPVQEELRFDAEPDSPVFR

SEQ ID NO: 126 - protein, 22 aa, Acidothiobacillus ferrooxidans
NVEAVPPEALQMNLLEEPVDLR

SEQ ID NO: 127 - protein, 17 aa, Legionella pneumophila
LKQENTYQSVQLPLLDL

SEQ ID NO: 128 - protein, 16 aa, Coxiella burnetii
SFSEDPLLELQRTFEW

SEQ ID NO: 129 - protein, 15 aa, Pseudomonas aeruginosa
RLLDLQGAHEQLRLF

SEQ ID NO: 130 - protein, 18 aa, Pseudomonas putida
RLRDLRGAHEQLELFPPK

SEQ ID NO: 131 - protein, 17 aa, Pseudomonas syringae
RLHDLRDAHEQLELFST

SEQ ID NO: 132 - protein, 17 aa, Pseudomonas fluorescens
RLEDLRGGFEQMELFER

SEQ ID NO: 133 - protein, 16 aa, Shewanella putrefaciens
LISEVDPLQTQLVLSI

SEQ ID NO: 134 - protein, 21 aa, Vibrio cholerae
VMLKPELQMKQLSMFPSDGWQ

SEQ ID NO: 135 - protein, 15 aa, Pasteurella multocida
PETTESKTQVQMSLW

SEQ ID NO: 136 - protein, 15 aa, Haemophilus influenzae
VNLPEENKQEQMSLW

SEQ ID NO: 137 - protein, 15 aa, Actinobacillus actinomycetemcomitans
VTLPEEKQSEQMSLW

SEQ ID NO: 138 - protein, 16 aa, Escherichia coli
VTLLDPQMERQLVLGL

SEQ ID NO: 139 - protein, 16 aa, Salmonella typhi
VTLLDPQLERQLVLGL

SEQ ID NO: 140 - protein, 16 aa, Salmonella typhimurium
VTLLDPQLERQLVLGL

SEQ ID NO: 141 - protein, 16 aa, Klebsiella pneumoniae
VTLLDPQLERQLLLGI

SEQ ID NO: 142 - protein, 17 aa, Yersinia pestis
VTLLDPQLERQLLLDWG

SEQ ID NO: 143 - protein, 26 aa, Desulfovibrio vulgaris
LGVSHFGGERQMSLPIGGMPRRDDTR

SEQ ID NO: 144 - protein, 25 aa, Geobacter sulfurreducens
AISNLVHASEQLPLFPEERRLTTLS

SEQ ID NO: 145 - protein, 25 aa, Geobacter sulfurreducens
RITNLCYQREQLPLFEKERRKALAT

SEQ ID NO: 146 - protein, 26 aa, Streptomyces coelicolor
SLTSAEHASHQLTFDPVDEKVRRIEE

SEQ ID NO: 147 - protein, 25 aa, Thermobifida fusca
GLVSADRVHHQLALDEEGPGWRAVE

SEQ ID NO: 148 - protein, 26 aa, Mycobacterium avium
VSGIDRDGAQQLMLPFEGRPPDAIDA

SEQ ID NO: 149 - protein, 25 aa, Mycobacterium avium
VGFSGLSEVRQESLFPDLEMPAPQS

SEQ ID NO: 150 - protein, 26 aa, Mycobacterium smegmatis
VSNIDRGGTQQLELPFAEQPDPVAID

SEQ ID NO: 151 - protein, 25 aa, Mycobacterium smegmatis
VGFSGLSDIRQESLFPDLEQPEEFP

SEQ ID NO: 152 - protein, 25 aa, Mycobacterium tuberculosis
VGFSGLSDIRQESLFADSDLTQETA

SEQ ID NO: 153 - protein, 25 aa, Corynebacterium diptheriae
VGLSGLEDARQDILFPELDRVVPVK

SEQ ID NO: 154 - protein, 26 aa, Dehalococcoides ethenogenes
GISDFCGPEKQLEIDPARARLEKLDA

SEQ ID NO: 155 - protein, 25 aa, Desulfitobacterium hafniense
TASRLQKGIEQLSLFQEESEEQTEL

SEQ ID NO: 156 - protein, 23 aa, Clostridium difficile
NLSDKKETYKDITLFEYMDSIQM

SEQ ID NO: 157 - protein, 25 aa, Carboxydothermus hydrogenoformans
TPLVPVGGGRQISLFGEDLRRENLY

SEQ ID NO: 158 - protein, 25 aa, Bacillus halodurans
DVIDKKYAYEPLDLFRYEEQIKQAT

SEQ ID NO: 159 - protein, 25 aa, Bacillus stearothermophilus
HVFDEREEGKQLDLFRYEEEAKVEE

SEQ ID NO: 160 - protein, 25 aa, Bacillus subtilis
DLVEKEQAYKQLDLFSFNEDAKDEP

SEQ ID NO: 161 - protein, 18 aa, Staphylococcus aureus
VGNLEQSTYKNMTIYDFI

SEQ ID NO: 162 - protein, 18 aa, Staphylococcus epidermidis
VGSLEQSDFKNLTIYDFI

SEQ ID NO: 163 - protein, 25 aa, Bacillus anthracis
EIEWKTESVKQLDLFSFEEDAKEEP

SEQ ID NO: 164 - protein, 17 aa, Listeria innocua
VTNLKPVYFENLRLEGL

SEQ ID NO: 165 - protein, 17 aa, Listeria monocytogenes
VTNLKPVYFENLRLEGL

SEQ ID NO: 166 - protein, 17 aa, Listeria monocytogenes
VTNLKPVYFENLRLEGL

SEQ ID NO: 167 - protein, 18 aa, Enterococcus faecalis
NLDPLAYENIVLPLWEKS

SEQ ID NO: 168 - protein, 20 aa, Enterococcus faecium
NLDPMTYENIVLPLWENQEI

SEQ ID NO: 169 - protein, 17 aa, Lactococcus lactis
GVTVTEFGAQKATLDMQ

SEQ ID NO: 170 - protein, 19 aa, Streptococcus equi
TMTGLKDKVTDILLDLSFN

SEQ ID NO: 171 - protein, 16 aa, Streptococcus pyogenes
TMTMLEDKVADISLDL

SEQ ID NO: 172 - protein, 21 aa, Streptococcus mutans
VTALEDSTREELSLTADDFKT

SEQ ID NO: 173 - protein, 16 aa, Ureaplasma urealyticum
KLVKKENVKKQLFLFD

SEQ ID NO: 174 - protein, 25 aa, Mycoplasma genitalium
LKKIDTDEGQKKSLFYQFIPKSISK

SEQ ID NO: 175 - protein, 25 aa, Mycoplasma pneumoniae
LKNNPSSSRPEGLLFYEYQQAKPKQ

SEQ ID NO: 176 - protein, 25 aa, Mycoplasma pulmonis
DFGDIYQSDLSFDLFDQKYDSKKEK

SEQ ID NO: 177 - protein, 25 aa, Clostridium acetobutylicum
LSGLCSGSSVQISMFDEKTDTRNEI

SEQ ID NO: 178 - protein, 25 aa, Fibrobacter succinogenes
ANNVLEATQESYDLFTDVKKIEREK

SEQ ID NO: 179 - protein, 25 aa, Bacillus halodurans
LSNLTSDEAWQLSFFGNRDRAHQLG

SEQ ID NO: 180 - protein, 25 aa, Bacillus subtilis
LSNIEDDVNQQLSLFEVDNEKRRKL

SEQ ID NO: 181 - protein, 25 aa, Bacillus subtilis
LSQLSSDDIWQLNLFQDYAKKMSLG

SEQ ID NO: 182 - protein, 25 aa, Staphylococcus aureus
LSQFINEDERQLSLFEDEYQRKRDE

SEQ ID NO: 183 - protein, 25 aa, Staphylococcus epidermidis
LTQFIKESDRQLNLFIDEYERKKDV

SEQ ID NO: 184 - protein, 25 aa, Bacillus anthracis
LTNLLQEGEEQISLFDNVTQREQEV

SEQ ID NO: 185 - protein, 25 aa, Bacillus anthracis
LTKLIGEGEEQISLFDNIIQREKEI

SEQ ID NO: 186 - protein, 25 aa, Listeria innocua
CGKLTLKTGLQLNLFEDATRTLNHE

SEQ ID NO: 187 - protein, 25 aa, Listeria innocua
CAGIKRKTSMQLSVFEDYTKTLQQE

SEQ ID NO: 188 - protein, 25 aa, Listeria monocytogenes
CGKITLKTGLQLNLFEDATRTLNHE

SEQ ID NO: 189 - protein, 25 aa, Listeria monocytogenes
CGKITLKTGLQLNLFEDFTQTLNHE

SEQ ID NO: 190 - protein, 25 aa, Enterococcus faecalis
YGRLVWNKNLQLDLFPVPEEQIHET

SEQ ID NO: 191 - protein, 25 aa, Enterococcus faecalis
YGKLVWNESLQLDLFSEPEEQISEM

SEQ ID NO: 192 - protein, 25 aa, Enterococcus faecalis
FGKLVWDTTLQIDLFSPPEEQIINN

SEQ ID NO: 193 - protein, 25 aa, Enterococcus faecium
CSDLVYATGLQLNLFEDPEKQINEA

SEQ ID NO: 194 - protein, 25 aa, Enterococcus faecium
CSKLVYSNALQLDLFEDPNEQVKDL

SEQ ID NO: 195 - protein, 25 aa, Lactococcus lactis
GNQLSDSSVKQLSLFESVQENQTNK

SEQ ID NO: 196 - protein, 25 aa, Lactococcus lactis
ANNLIDEPYQLISLFDSDEENEETI

SEQ ID NO: 197 - protein, 25 aa, Streptococcus gordonii
YSDFVDQEYGLISLFDDPLQVQKEE

SEQ ID NO: 198 - protein, 25 aa, Streptococcus gordonii
GNQLSDSSVKQLSLFESVQENQTNK

SEQ ID NO: 199 - protein, 25 aa, Streptococcus pneumoniae
YSGLVDESFGLISLFDDIEKIEKEE

SEQ ID NO: 200 - protein, 25 aa, Magnetospirillum magnetotacticum
AEEVVPAGAEQPRLWGASSGEDARA

SEQ ID NO: 201 - protein, 25 aa, Methylobacterium extorquens
ASRVEPLAERQNSHLAAGQQAPDLA

SEQ ID NO: 202 - protein, 25 aa, Rhodopseudomonas palustris
ASVSVAVTEAQRGFDTTAHQAEDVA

SEQ ID NO: 203 - protein, 25 aa, Mesorhizobium loti
VLAAAAFDMAQADLTGEVTDDGADI

SEQ ID NO: 204 - protein, 25 aa, Brucella suis
ALRSSTVAQRQTGLDQHEEDEAGFS

SEQ ID NO: 205 - protein, 25 aa, Sinorhizobium meliloti
VLRSERLDPAQQDFSGAPDESQLLA

SEQ ID NO: 206 - protein, 25 aa, Agrobacterium tumefaciens
AVMTEPLEEAQKASALIGDDVTDVT

SEQ ID NO: 207 - protein, 25 aa, Agrobacterium tumefaciens
ATHAEPLVAAQARSSLLDEGRAEIA

SEQ ID NO: 208 - protein, 25 aa, Agrobacterium tumefaciens
AVMAEPLEERQKSSSLVEDEVTDVT

SEQ ID NO: 209 - protein, 25 aa, Caulobacter crescentus
AFAVEPMAAAQARLDADAAASADET

SEQ ID NO: 210 - protein, 25 aa, Rhodobacter capsulatus
ATRVEPLAPAQLGTTPAASPDRLAD

SEQ ID NO: 211 - protein, 25 aa, Sphingomonas aromaticivorans
LPVTEPLAASQPTLDGSGQETTEVA

SEQ ID NO: 212 - protein, 25 aa, Bordetella bronchiseptica
APDTVPQPAASTCLFPEPGGTPADH

SEQ ID NO: 213 - protein, 25 aa, Bordetella parapertussis
APDTVPQPAASTCLFPEPGGTPADH

SEQ ID NO: 214 - protein, 25 aa, Burkholderia pseudomallei
ATRVESVAPPADDLFPEPGGTREAR

SEQ ID NO: 215 - protein, 25 aa, Burkholderia cepacia
ADQVGEYAGQSDTLFPMPESDGDSI

SEQ ID NO: 216 - protein, 25 aa, Burkholderia mallei
ATRIESVAPPADDLFPEPGGTREAR

SEQ ID NO: 217 - protein, 25 aa, Ralstonia metallidurans
VEAMEICVPQSDSLFPEPGAEPAEL

SEQ ID NO: 218 - protein, 25 aa, Acidothiobacillus ferrooxidans
ALAPQHWPGRQATWWQDGVEEARWQ

SEQ ID NO: 219 - protein, 25 aa, Methylococcus capsulatus
SADIQPFTLPTADLFTPGAAGGESW

SEQ ID NO: 220 - protein, 25 aa, Pseudomonas aeruginosa
ARELPPFTPQHRELFDERPQQYLGW

SEQ ID NO: 221 - protein, 25 aa, Pseudomonas putida
AEDLPPFVPQHRELFDERPQQYLGW

SEQ ID NO: 222 - protein, 25 aa, Pseudomonas syringae
ARDLPDFVPAHRELFDERVQQTLPW

SEQ ID NO: 223 - protein, 25 aa, Pseudomonas fluorescens
AEDLPSFVPQFQELFDDRPQQTLPW

SEQ ID NO: 224 - protein, 19 aa, Mycobacterium avium
AVEVVSAEALQLPLWGGLG

SEQ ID NO: 225 - protein, 25 aa, Mycobacterium smegmatis
PVEVVSSAALQLPLWGGIGEEDRLR

SEQ ID NO: 226 - protein, 25 aa, Mycobacterium tuberculosis
VETVSASEGLQLPLWGGLGEQDRLR

SEQ ID NO: 227 - protein, 25 aa, Corynebacterium diptheriae
LRPYECMRPSQPQLWGTNKSDEESE

SEQ ID NO: 228 - protein, 25 aa, Corynebacterium glutamicum
PLECVPPDMASGGLWDTGRSQQHVA

SEQ ID NO: 229 - protein, 25 aa, Magnetococcus sp.
LLFLVSAQHFQPSLFAPPPRLPNSR

SEQ ID NO: 230 - protein, 25 aa, Porphyromonas gingivalis
ILSDLVAEAYQLNLFDPIDRMRQER

SEQ ID NO: 231 - protein, 25 aa, Bacteroides fragilis
VIITEITDSTQLGLFDSVDREKRKR

SEQ ID NO: 232 - protein, 25 aa, Cytophaga hutchinsonii
VSGIVPEDRVQQNLFDTVDRSKHNK

SEQ ID NO: 233 - protein, 25 aa, Cytophaga hutchinsonii
VIDIVPEEKIQLNLFEPQKNARLHA

SEQ ID NO: 234 - protein, 25 aa, Prochlorococcus marinus
MQDLTNCKYLQQSIINYESQEESKK

SEQ ID NO: 235 - protein, 25 aa, Prochlorococcus marinus
MQNLQSADHLQQHLLVAVHADEQHR

SEQ ID NO: 236 - protein, 25 aa, Synechococcus sp.
MQHLQGTELLQSHLLVPLSEAQQQR

SEQ ID NO: 237 - protein, 25 aa, Methylobacterium extorquens
STDLVPLEASQRALIGAFDRERGGA

SEQ ID NO: 238 - protein, 25 aa, Acidothiobacillus ferrooxidans
LLEITSADALQADLFLSAEEEARAH

SEQ ID NO: 239 - protein, 16 aa, Legionella pneumophila
LEDLIPKKPRQLDMFH

SEQ ID NO: 240 - protein, 25 aa, Legionella pneumophila
LGDLIEKNCLQLDLFNQVSEKELNQ

SEQ ID NO: 241 - protein, 25 aa, Pseudomonas syringae
LMDICQPGEFTDDLFTIDQPASADR

SEQ ID NO: 242 - protein, 25 aa, Shewanella putrefaciens
LGDFYAPGVFQLGLFDEAKPQPKSK

SEQ ID NO: 243 - protein, 25 aa, Shewanella putrefaciens
LIELMPTKHIQYDLFHAPTENPALM

SEQ ID NO: 244 - protein, 25 aa, Morganella morganii
MLSDLQGYETQLDLFSPAAVRPGSE

SEQ ID NO: 245 - protein, 25 aa, Providencia rettgeri
LSDFYDPGMFQPGLFDDVSTRSNSQ

SEQ ID NO: 246 - protein, 25 aa, Escherichia coli
MLADFSGKEAQLDLFDSATPSAGSE

SEQ ID NO: 247 - protein, 25 aa, Escherichia coli
LGDFFSQGVAQLNLFDDNAPRPGSE

SEQ ID NO: 248 - protein, 25 aa, Shigella flexneri
LADFTPSGIAQPGLFDEIQPRKNSE

SEQ ID NO: 249 - protein, 25 aa, Salmonella typhi
MLSSMTDGTEQLSLFDERPARRGSE

SEQ ID NO: 250 - protein, 25 aa, Salmonella typhi
LNDFTPTGISQLNLFDEVQPHERSE

SEQ ID NO: 251 - protein, 25 aa, Salmonella typhi
LGGFFSQGVAQLNLFDDNAPRAGSA

SEQ ID NO: 252 - protein, 25 aa, Salmonella typhimurium
LADFTPSGIAQPGLFDEIQPRKNSE

SEQ ID NO: 253 - protein, 25 aa, Salmonella typhimurium
MLADFSGKEAQLDLFDSATPSAGSE

SEQ ID NO: 254 - protein, 25 aa, Salmonella typhimurium
LNDFTPTGVSQLNLFDEVQPRERSE

SEQ ID NO: 255 - protein, 25 aa, Salmonella typhimurium
LGDFFSQGVAQLNLFDDNAPRAGSA

SEQ ID NO: 256 - protein, 25 aa, Klebsiella pneumoniae
LNDFTGSGVSQLQLFDERPPRPHSA

SEQ ID NO: 257 - protein, 25 aa, Klebsiella pneumoniae
LGDFYSQGVAQLNLFDDNAPRKGSE

SEQ ID NO: 258 - protein, 25 aa, Klebsiella pneumoniae
LGDFYSQGVAQLNLFDELAPRHNSA

SEQ ID NO: 259 - protein, 25 aa, Serratia marcescens
MLSDLQGHETQLDLFAPAAVRPGSE

SEQ ID NO: 260 - protein, 25 aa, Desulfovibrio vulgaris
LFGLEPAAGRQGSLLDLLDGSHEHK

SEQ ID NO: 261 - protein, 22 aa, Magnetococcus sp.
MHTGSAQLLIAFPLDPVLSWEN

SEQ ID NO: 262 - protein, 20 aa, Magnetospirillum magnetotacticum
MSEAQLPLAFGHVPSLAAED

SEQ ID NO: 263 - protein, 20 aa, Rhodopseudomonas palustris
VEPRQLALDLPHAESLSRED

SEQ ID NO: 264 - protein, 26 aa, Mesorhizobium loti
MTAQRTDPPRQLPLDLGHGTGYSRDE

SEQ ID NO: 265 - protein, 23 aa, Sinorhizobium meliloti
MKRHLSEQLPLVFGHAPATGRDD

SEQ ID NO: 266 - protein, 26 aa, Agrobacterium tumefaciens
KTDNARSKAEQLPLAFSHQSASGRED

SEQ ID NO: 267 - protein, 19 aa, Caulobacter crescentus
MSTQFKLPLASPLTHGRED

SEQ ID NO: 268 - protein, 19 aa, Rhodobacter sphaeroides
VKGQLAFDLPIRPALSRED

SEQ ID NO: 269 - protein, 19 aa, Rhodobacter capsulatus
MTRQLPLPLPVRVAEGRED

SEQ ID NO: 270 - protein, 18 aa, Rickettsia conorii
VQQYIFRFTTSSKYHPDE

SEQ ID NO: 271 - protein, 18 aa, Rickettsia prowazekii
MQQYIFHFTPSNKYHPDE

SEQ ID NO: 272 - protein, 25 aa, Wolbachia sp.
RKRLRKRFNVQLNLFNNNQADYSRQ

SEQ ID NO: 273 - protein, 18 aa, Neisseria gonorrhoeae
MNQLIFDFAAHDYPSFDK

SEQ ID NO: 274 - protein, 18 aa, Neisseria meningitidis
MNQLIFDFAAHDYPSFDK

SEQ ID NO: 275 - protein, 18 aa, Nitrosomonas europaea
MRQQLLDITEIGPPSLDN

SEQ ID NO: 276 - protein, 19 aa, Bordetella parapertussis
MNRQLLLDVLPAPAPTLNN

SEQ ID NO: 277 - protein, 19 aa, Burkholderia fungorum
VLRQLTLDLGTPPPSTFDN

SEQ ID NO: 278 - protein, 19 aa, Burkholderia pseudomallei
VTRQLTLDLGTPPPSTFDN

SEQ ID NO: 279 - protein, 19 aa, Burkholderia mallei
VTRQLTLDLGTPPPSTFDN

SEQ ID NO: 280 - protein, 22 aa, Ralstonia metallidurans
MSPRQKQLSLELGSPPPSTFEN

SEQ ID NO: 281 - protein, 20 aa, Acidothiobacillus ferrooxidans
MGNRQRILPLGVQAPATLEG

SEQ ID NO: 282 - protein, 20 aa, Xylella fastidiosa
MSVSQLPLALRYSSDQRFET

SEQ ID NO: 283 - protein, 19 aa, Legionella pneumophila
MNKQLALAIKLNDEATLDD

SEQ ID NO: 284 - protein, 19 aa, Coxiella burnetii
MIDQLPLRVQLREETTFAN

SEQ ID NO: 285 - protein, 19 aa, Methylococcus capsulatus
MAQQIPLHFAVDPLQTFEA

SEQ ID NO: 286 - protein, 20 aa, Pseudomonas aeruginosa
MKPIQLPLSVRLRDDATFAN

SEQ ID NO: 287 - protein, 21 aa, Pseudomonas putida
MKPPIQLPLGVRLRDDATFIN

SEQ ID NO: 288 - protein, 20 aa, Pseudomonas syringae
MKPIQLPLSVRLRDDATFVN

SEQ ID NO: 289 - protein, 20 aa, Pseudomonas fluorescens
MKPIQLPLGVRLRDDATFIN

SEQ ID NO: 290 - protein, 26 aa, Shewanella putrefaciens
DVRVPLNSPLQLSLPVYLPDDETFNS

SEQ ID NO: 291 - protein, 26 aa, Pasteurella multocida
FVGCFLLENFQLPLPIHQLDDETLDN

SEQ ID NO: 292 - protein, 19 aa, Haemophilus influenzae
MNKQLPLPIHQIDDATLEN

SEQ ID NO: 293 - protein, 26 aa, Haemophilus ducreyi
NWSIRFKNSLQLLLPIHQIDDETLDS

SEQ ID NO: 294 - protein, 22 aa, Actinobacillus actinomycetemcomitans
MSEPHFQLPLPIHQLDDDTLEN

SEQ ID NO: 295 - protein, 25 aa, Escherichia coli
VEVSLNTPAQLSLPLYLPDDETFAS

SEQ ID NO: 296 - protein, 25 aa, Salmonella typhi
VEVSLNTPAQLSLPLYLPDDETFAS

SEQ ID NO: 297 - protein, 25 aa, Salmonella typhimurium
VEVSLNTPAQLSLPLYLPDDETFAS

SEQ ID NO: 298 - protein, 26 aa, Yersinia pestis
MVEVLLNTPAQLSLPLYLPDDETFAS

SEQ ID NO: 299 - protein, 26 aa, Geobacter sulfurreducens
ARSSRPFPAMQLVFDFPVTPKYSFDN

SEQ ID NO: 300 - protein, 16 aa, Nostoc punctiforme
PWNNLEHPPNQLSLWS

SEQ ID NO: 301 - protein, 15 aa, Anabaena sp.
PWNHLDYPPHQLNLW

SEQ ID NO: 302 - protein, 15 aa, Pseudomonas aeruginosa
PEPIPAPEVEQLGLL

SEQ ID NO: 303 - protein, 15 aa, Pseudomonas putida
PELPRAPEVEQLGLL

SEQ ID NO: 304 - protein, 15 aa, Pseudomonas syringae
PELDRGPQVEQLGLL

SEQ ID NO: 305 - protein, 15 aa, Pseudomonas fluorescens
PELYREPAAEQLGLL

SEQ ID NO: 306 - protein, 16 aa, Shewanella putrefaciens
LDKKPEETSTQMGLSW

SEQ ID NO: 307 - protein, 15 aa, Vibrio cholerae
APFPVTPEQPQLSMF

SEQ ID NO: 308 - protein, 15 aa, Pasteurella multocida
VKPKPEFLTGQQSLF

SEQ ID NO: 309 - protein, 15 aa, Escherichia coli
EIGAVPAIPQQSSLF

SEQ ID NO: 310 - protein, 15 aa, Salmonella typhi
EIGTAPSIPQQSSLF

SEQ ID NO: 311 - protein, 15 aa, Salmonella typhimurium
EIGTAPSIPQQSSLF

SEQ ID NO: 312 - protein, 15 aa, Yersinia pestis
TLPTAPDWPEQETLF

SEQ ID NO: 313 - protein, 16 aa, Bacillus halodurans
EIEYRGLTPKQLNLFE

SEQ ID NO: 314 - protein, 15 aa, Bacillus stearothermophilus
GIEYTGLAPRQLGLF

SEQ ID NO: 315 - protein, 15 aa, Bacillus subtilis
DIEYSGLAPRQLDLF

SEQ ID NO: 316 - protein, 15 aa, Staphylococcus aureus
NIEYEGLAPQQLKLF

SEQ ID NO: 317 - protein, 15 aa, Staphylococcus epidermidis
DIDYEGLAPQQLKLF

SEQ ID NO: 318 - protein, 16 aa, Bacillus anthracis
NITYGEPKPEQLNLFE

SEQ ID NO: 319 - protein, 17 aa, Listeria innocua
QVEFQGLAPMQMDLFSE

SEQ ID NO: 320 - protein, 17 aa, Listeria monocytogenes
QVEFQGLAPMQMDLFSE

SEQ ID NO: 321 - protein, 15 aa, Pediococcus acidilactici
GIHFTGLGPMQLDLF

SEQ ID NO: 322 - protein, 15 aa, Enterococcus faecalis
NLSYDDLNPKQLDLF

SEQ ID NO: 323 - protein, 15 aa, Enterococcus faecium
NIKPDGLNPTQMDLF

SEQ ID NO: 324 - protein, 25 aa, Magnetococcus sp.
QGHAPASQPYQLTLFEDAPPSPALL

SEQ ID NO: 325 - protein, 25 aa, Aquifex aeolicus
RELEEKENKKEDIVPLLEETFKKSE

SEQ ID NO: 326 - protein, 25 aa, Aquifex pyrophilus
LKELEGEKGKQEVLPFLEETYKKSV

SEQ ID NO: 327 - protein, 17 aa, Thermotoga maritima
KNGKSNRFSQQIPLFPV

SEQ ID NO: 328 - protein, 25 aa, Chloroflexus aurantiacus
VPAQETGQGMQLSFFDLAPHPVVEY

SEQ ID NO: 329 - protein, 25 aa, Porphyromonas gingivalis
DEKGRSIDGYQLSFFQLDDPVLSQI

SEQ ID NO: 330 - protein, 25 aa, Bacteroides fragilis
AEVSENRGGMQLSFFQLDDPILCQI

SEQ ID NO: 331 - protein, 25 aa, Cytophaga hutchinsonii
KLKEVPKSTLQMSLFEAADPAWDSI

SEQ ID NO: 332 - protein, 25 aa, Chlorobium tepidum
QALPLRVESRQISLFEEEESRLRKA

SEQ ID NO: 333 - protein, 15 aa, Chlamydia trachomatis
DLRPEPEKAQQLVMF

SEQ ID NO: 334 - protein, 15 aa, Chlamydophila pneumoniae
ITRPAQDKMQQLTLF

SEQ ID NO: 335 - protein, 17 aa, Synechocystis sp.
AAEAAEDQAKQLDIFGF

SEQ ID NO: 336 - protein, 25 aa, Fibrobacter succinogenes
AQNKKIKAQPQMDLFAPPDENTLLL

SEQ ID NO: 337 - protein, 25 aa, Treponema denticola
EKTPSSPAEKGLSLFPEEELILNEI

SEQ ID NO: 338 - protein, 25 aa, Treponema pallidum
AASKPCAQRVSADLFTQEELIGAEI

SEQ ID NO: 339 - protein, 25 aa, Borrelia burgdorferi
VGREGNSCLEFLPHVSSDGNDKEIL

SEQ ID NO: 340 - protein, 25 aa, Magnetospirillum magnetotacticum
QASGMARLADDLPLFAALAKPVAAS

SEQ ID NO: 341 - protein, 25 aa, Magnetospirillum magnetotacticum
RERPTRRRIEDLPLFASLAAAPPPP

SEQ ID NO: 342 - protein, 25 aa, Rhodopseudomonas palustris
DRGQPKTLIDDLPLFAITARAPAEA

SEQ ID NO: 343 - protein, 25 aa, Mesorhizobium loti
VSGKTNRLVDDLPLFSVAMKREAPK

SEQ ID NO: 344 - protein, 25 aa, Brucella suis
TSGKADRLIDDLPLFSVMLQQEKPK

SEQ ID NO: 345 - protein, 25 aa, Sinorhizobium meliloti
RKNPASQLIDDLPLFQVAVRREEAA

SEQ ID NO: 346 - protein, 25 aa, Agrobacterium tumefaciens
RKNPASQLIDDLPLFQIAVRREETR

SEQ ID NO: 347 - protein, 25 aa, Caulobacter crescentus
SKDQSPAKLDDLPLFAVSQAVAVTS

SEQ ID NO: 348 - protein, 25 aa, Rhodobacter sphaeroides
SGGRRQTLIDDLPLFRAAPPPPAPA

SEQ ID NO: 349 - protein, 25 aa, Rickettsia conorii
GKNILSTESNNLSLFYLEPNKTTIS

SEQ ID NO: 350 - protein, 25 aa, Rickettsia prowazekii
EKNILSNASNNLSLFNFEHEKPISN

SEQ ID NO: 351 - protein, 25 aa, Sphingomonas aromaticivorans
ATGGLAAGLDDLPLFAAAIEAAEEK

SEQ ID NO: 352 - protein, 25 aa, Neisseria gonorrhoeae
LENQAAANRPQLDIFSTMPSEKGDE

SEQ ID NO: 353 - protein, 25 aa, Neisseria meningitidis
LENQAAANRPQLDIFSTMPSEKGDE

SEQ ID NO: 354 - protein, 25 aa, Nitrosomonas europaea
LEQETLSRSPQQTLFETVEENAKAV

SEQ ID NO: 355 - protein, 25 aa, Bordetella bronchiseptica
RLEAQGAPTPQLGLFAAALDADVQS

SEQ ID NO: 356 - protein, 25 aa, Bordetella pertussis
RLEAQGAPTPQLGLFAAALDADVQS

SEQ ID NO: 357 - protein, 25 aa, Burkholderia pseudomallei
EQQSAAQATPQLDLFAAPPVVDEPE

SEQ ID NO: 358 - protein, 25 aa, Burkholderia cepacia
EQQSAAQPAPQLDLFAAPMPMLLED

SEQ ID NO: 359 - protein, 25 aa, Burkholderia mallei
EQQSAAQATPQLDLFAAPPVVDEPE

SEQ ID NO: 360 - protein, 25 aa, Ralstonia metallidurans
EQSADATPTPQMDLFSAQSSPSADD

SEQ ID NO: 361 - protein, 25 aa, Acidothiobacillus ferrooxidans
RSSLSHTAPAQLSLFQAAPHPAVYR

SEQ ID NO: 362 - protein, 25 aa, Xylella fastidiosa
ITPLALDAPQQCSLFASAPSAAQEA

SEQ ID NO: 363 - protein, 25 aa, Xylella fastidiosa
ITPLALDAPQQCSLFASAPSAAQEA

SEQ ID NO: 364 - protein, 25 aa, Xylella fastidiosa
ITPLALDAPQQCSLFASAPSAAQEA

SEQ ID NO: 365 - protein, 25 aa, Legionella pneumophila
QIQDTQSILVQTQIIKPPTSPVLTE

SEQ ID NO: 366 - protein, 25 aa, Coxiella burnetii
PVISETQQPQQNELFLPIENPVLTQ

SEQ ID NO: 367 - protein, 25 aa, Methylococcus capsulatus
SAHQQAAPVAQLDLFLPPVVDEPEC

SEQ ID NO: 368 - protein, 25 aa, Pseudomonas aeruginosa
QQSGKPASPMQSDLFASLPHPVIDE

SEQ ID NO: 369 - protein, 25 aa, Azotobacter vinelandii
REAGKPQPPIQSDLFASLPHPLMEE

SEQ ID NO: 370 - protein, 25 aa, Pseudomonas putida
KAKDAPQVPHQSDLFASLPHPAIEK

SEQ ID NO: 371 - protein, 25 aa, Pseudomonas syringae
AKPGKPAIPQQSDMFASLPHPVLDE

SEQ ID NO: 372 - protein, 25 aa, Pseudomonas fluorescens
AAKGKPAAPQQSDMFASLPHPVLDE

SEQ ID NO: 373 - protein, 25 aa, Shewanella putrefaciens
HQVEGTKTPIQTLLALPEPVENPAV

SEQ ID NO: 374 - protein, 25 aa, Vibrio parahaemolyticus
PRPSTVDVANQLSLIPEPSEIEQAL

SEQ ID NO: 375 - protein, 25 aa, Vibrio cholerae
RKPSRVDIANQLSLIPEPSAVEQAL

SEQ ID NO: 376 - protein, 25 aa, Pasteurella multocida
DLRQLNQTQGELALMEEDDSKTAVW

SEQ ID NO: 377 - protein, 25 aa, Haemophilus influenzae
IQDLRLLNQRQGELFFEQETDALRE

SEQ ID NO: 378 - protein, 25 aa, Haemophilus ducreyi
QQTKMAQQHPQADLLFTVEMPEEEK

SEQ ID NO: 379 - protein, 25 aa, Actinobacillus actinomycetemcomitans
IQDLRLLNQRQGELAFESAEDENKD

SEQ ID NO: 380 - protein, 25 aa, Escherichia coli
NAAATQVDGTQMSLLSVPEETSPAV

SEQ ID NO: 381 - protein, 25 aa, Salmonella enteritidis
NAAATQVDGTQMSLLAAPEETSPAV

SEQ ID NO: 382 - protein, 25 aa, Salmonella typhi
NAAATQVDGTAMSLLAAPEETSPAV

SEQ ID NO: 383 - protein, 25 aa, Salmonella typhimurium
NAAATQVDGTQMSLLAAPEETSPAV

SEQ ID NO: 384 - protein, 25 aa, Yersinia pestis
NAAASTIDGSQMTLLNEEIPPAVEA

SEQ ID NO: 385 - protein, 25 aa, Yersinia pseudotuberculosis
NAAASTIDGSQMTLLNEEIPPAVEA

SEQ ID NO: 386 - protein, 25 aa, Geobacter sulfurreducens
KRAGAPKPSPQLSLFDQGDDLLRRR

SEQ ID NO: 387 - protein, 25 aa, Desulfitobacterium hafniense
EHLLNKEKATQLSLFEVQPLDPLLQ

SEQ ID NO: 388 - protein, 25 aa, Clostridium difficile
EDSVKEVALTQISFDSVNRDILSEE

SEQ ID NO: 389 - protein, 25 aa, Carboxydothermus hydrogenoformans
GLKVKDTVPVQLSLFEEKPEPSGVI

SEQ ID NO: 390 - protein, 25 aa, Bacillus halodurans
KEVASTNEPTQLSLFEPEPLEAYKP

SEQ ID NO: 391 - protein, 25 aa, Bacillus stearothermophilus
EGVLAEAAFEQLSMFPDLAPAPVEP

SEQ ID NO: 392 - protein, 25 aa, Bacillus subtilis
QKPQVKEEPAQLSFFDEAEKPAETP

SEQ ID NO: 393 - protein, 25 aa, Staphylococcus aureus
TLSQKDFEQASFDLFENDQKSEIEL

SEQ ID NO: 394 - protein, 25 aa, Staphylococcus epidermidis
HTSNHNYEQATFDLFDGYNQQSEVE

SEQ ID NO: 395 - protein, 25 aa, Bacillus anthracis
ETKVDNEEESQLSFFGAEQSSKKQD

SEQ ID NO: 396 - protein, 25 aa, Listeria innocua
KQPEEIHEEVQLSMFPVEPEEKASS

SEQ ID NO: 397 - protein, 25 aa, Listeria monocytogenes
KQPEEVHEEVQLSMFPLEPEKKASS

SEQ ID NO: 398 - protein, 25 aa, Enterococcus faecalis
EVSEVHEETEQLSLFKEVSTEELSV

SEQ ID NO: 399 - protein, 25 aa, Enterococcus faecium
IQDRVKEENQQLSLFSELSENETEV

SEQ ID NO: 400 - protein, 25 aa, Streptococcus equi
VRETQQLANQQLSLFTDDGSSSEII

SEQ ID NO: 401 - protein, 25 aa, Streptococcus pyogenes
VESSSAVRQGQLSLFGDEEKAHEIR

SEQ ID NO: 402 - protein, 25 aa, Streptococcus mutans
ETKESQPVEEQLSLFAIDNNYEELI

SEQ ID NO: 403 - protein, 25 aa, Streptococcus pneumoniae
PMRQTSAVTEQISLFDRAEEHPILA

SEQ ID NO: 404 - protein, 25 aa, Clostridium acetobutylicum
VKEEPKKDSYQIDFNYLERESILKE

SEQ ID NO: 405 - protein, 25 aa, Chlorobium tepidum
KPQDFSSIFSADTLFAFSPEGIKVI

SEQ ID NO: 406 - protein, 15 aa, Anabaena sp.
APTTLESNKRQLSLF

SEQ ID NO: 407 - protein, 15 aa, Burkholderia cepacia
RDDFTALMSGQKPLF

SEQ ID NO: 408 - protein, 17 aa, Ralstonia metallidurans
DDDFETLLTGQMTLFPQ

SEQ ID NO: 409 - protein, 15 aa, Pseudomonas aeruginosa
GDDFATLVDRQMALF

SEQ ID NO: 410 - protein, 15 aa, Pseudomonas putida
GDDFARLTDHQLLLF

SEQ ID NO: 411 - protein, 15 aa, Pseudomonas syringae
DDDFSTLIGGQLGLF

SEQ ID NO: 412 - protein, 15 aa, Pseudomonas fluorescens
DDDFSTLIGGQLGLF

SEQ ID NO: 413 - protein, 15 aa, Shewanella putrefaciens
KLNYTNIASKQLSLI

SEQ ID NO: 414 - protein, 15 aa, Vibrio cholerae
GKQFDELIAPQLGLF

SEQ ID NO: 415 - protein, 15 aa, Escherichia coli
EDNFATLMTGQLGLF

SEQ ID NO: 416 - protein, 15 aa, Salmonella typhi
EDNFATLLTGQLGLF

SEQ ID NO: 417 - protein, 15 aa, Salmonella typhimurium
EDNFATVLTGQLGLF

SEQ ID NO: 418 - protein, 15 aa, Klebsiella pneumoniae
NDNFATIVTGQLGLF

SEQ ID NO: 419 - protein, 15 aa, Yersinia pestis
QDDFTTLITGQMGLF

SEQ ID NO: 420 - protein, 16 aa, Geobacter sulfurreducens
MKKFAPFLPRERTLFD

SEQ ID NO: 421 - protein, 25 aa, Magnetococcus sp.
TQHQKDQKLGFMNLFGDEEAENSES

SEQ ID NO: 422 - protein, 25 aa, Aquifex aeolicus
ANSEKALMATQNSLFGAPKEEVEEL

SEQ ID NO: 423 - protein, 25 aa, Thermotoga maritima
NKRVEKDILEIRSLFGEKVEQESSN

SEQ ID NO: 424 - protein, 25 aa, Chloroflexus aurantiacus
IEAQKAREIGQSSLFDIFGEATTAN

SEQ ID NO: 425 - protein, 25 aa, Thermus aquaticus
AETRERGRSGLVGLFAEVEEPPLVE

SEQ ID NO: 426 - protein, 25 aa, Deinococcus radiodurans
AEINARAQSGMSMMFGMEEVKKERP

SEQ ID NO: 427 - protein, 25 aa, Porphyromonas gingivalis
SVVQEEKHSQSNSLFGEEEDLMIPR

SEQ ID NO: 428 - protein, 25 aa, Bacteroides fragilis
NRYQADKAAAVNSLFGGDNVIDIAT

SEQ ID NO: 429 - protein, 25 aa, Cytophaga hutchinsonii
NAFQTEDDSNQSSLFGDSSSAKPAP

SEQ ID NO: 430 - protein, 25 aa, Chlorobium tepidum
QIQNKAVTLGQGGFFNDDFSDGQAG

SEQ ID NO: 431 - protein, 25 aa, Chlamydia trachomatis
SREKKEAATGVLTFFSLDSMARDPV

SEQ ID NO: 432 - protein, 25 aa, Chlamydophila pneumoniae
AKDKKEAASGVMTFFTLGAMDRKNE

SEQ ID NO: 433 - protein, 25 aa, Nostoc punctiforme
QSRAKDRASGQGNLFDLLGDGFSST

SEQ ID NO: 434 - protein, 25 aa, Anabaena sp.
QSRARDRASGQGNLFDLLGGYSSTN

SEQ ID NO: 435 - protein, 25 aa, Synechocystis sp.
QKRAKEKETGQLNIFDSLTAGESIK

SEQ ID NO: 436 - protein, 25 aa, Prochlorococcus marinus
SSRNRDRISGQGNLFDSISKNDTKE

SEQ ID NO: 437 - protein, 25 aa, Prochlorococcus marinus
ASRARDRLSGQGNLFDLVAGAADEQ

SEQ ID NO: 438 - protein, 25 aa, Synechococcus sp.
SSRAKDRDSGQGNLFDLMAAPNDED

SEQ ID NO: 439 - protein, 25 aa, Treponema denticola
SQKKENESTGQGSLFEGSGIKEFSD

SEQ ID NO: 440 - protein, 25 aa, Treponema pallidum
ARKKAVTSSRQASLFDETDLGECSE

SEQ ID NO: 441 - protein, 25 aa, Borrelia burgdorferi
SEDKNNKKLGQNSLFGALESQDPIQ

SEQ ID NO: 442 - protein, 25 aa, Magnetospirillum magnetotacticum
AQAAEDRQSSQMSLLGGSNAPTLKL

SEQ ID NO: 443 - protein, 25 aa, Rhodopseudomonas palustris
QRNHEAATSGQNDMFGGLSDAPSII

SEQ ID NO: 444 - protein, 25 aa, Mesorhizobium loti
SLAQQNAVSGQADIFGASLGAQSQA

SEQ ID NO: 445 - protein, 25 aa, Brucella suis
QRTQENAVSGQSDIFGLSGAPRETL

SEQ ID NO: 446 - protein, 25 aa, Sinorhizobium meliloti
QRAQENKVSGQSDMFGAGAATGPEK

SEQ ID NO: 447 - protein, 25 aa, Agrobacterium tumefaciens
QMAQNNRTIGQSDMFGSGGGTGPEK

SEQ ID NO: 448 - protein, 25 aa, Caulobacter crescentus
QSCHADRQGGQGGLFGSDPGAGRPR

SEQ ID NO: 449 - protein, 25 aa, Rhodobacter sphaeroides
AAIHEALNSSQVSLFGEAGADIPEP

SEQ ID NO: 450 - protein, 25 aa, Rhodobacter capsulatus
AAVAEAKSSAQVSLFGEAGDDLPPR

SEQ ID NO: 451 - protein, 25 aa, Rickettsia conorii
TAYHEEQESNQFSLIKVSSLSPTIL

SEQ ID NO: 452 - protein, 25 aa, Rickettsia helvetica
TSYHEEQESNQLSLIKVSSLSPTIL

SEQ ID NO: 453 - protein, 25 aa, Rickettsia prowazekii
TSYHQEQESNQFSLIKVSSLSPTIL

SEQ ID NO: 454 - protein, 25 aa, Rickettsia rickettsii
TAYHEEQESNQFSLIKVSSLSPTIL

SEQ ID NO: 455 - protein, 25 aa, Cowdria ruminantium
EYNKYNSSFNQISLFNDKNHYKLVE

SEQ ID NO: 456 - protein, 25 aa, Wolbachia sp.
NKNKQDKESSQAALFGSLDVLKPKL

SEQ ID NO: 457 - protein, 25 aa, Sphingomonas aromaticivorans
EEASRSRTSGQGGLFGGDDHATPAT

SEQ ID NO: 458 - protein, 25 aa, Neisseria gonorrhoeae
NADQKAANANQGGLFDMMEDAIEPV

SEQ ID NO: 459 - protein, 25 aa, Neisseria meningitidis
NADQKAANANQGGLFDMMEDAIEPV

SEQ ID NO: 460 - protein, 25 aa, Nitrosomonas
YAEQCSLAASQVSLFDENTDLIQPP

SEQ ID NO: 461 - protein, 25 aa, Bordetella bronchiseptica
AAEQAARSANQSSLFGDDSGDVVAG

SEQ ID NO: 462 - protein, 25 aa, Bordetella pertussis
AAEQAARSANQSSLFGDDSGDVVAG

SEQ ID NO: 463 - protein, 25 aa, Burkholderia pseudomallei
AAEQAAANALQAGLFDIGGVPAHQH

SEQ ID NO: 464 - protein, 25 aa, Burkholderia cepacia
AAEQASANALQAGLFDMGDAPSQGH

SEQ ID NO: 465 - protein, 25 aa, Burkholderia mallei
AAEQAAANALQAGLFDIGGVPAHQH

SEQ ID NO: 466 - protein, 25 aa, Ralstonia metallidurans
LDRTEGESANQVSLFDLMDDAGASH

SEQ ID NO: 467 - protein, 25 aa, Acidothiobacillus ferrooxidans
AQFQSSQASLQESLFSGQEALRVAP

SEQ ID NO: 468 - protein, 25 aa, Xylella fastidiosa
EQMSRERESGQNPLFGNADPSTPAI

SEQ ID NO: 469 - protein, 25 aa, Xylella fastidiosa
EQMSRERESGQNSLFGNADPGTPAI

SEQ ID NO: 470 - protein, 25 aa, Xylella fastidiosa
EQMSRERESGQNSLFGNADPGTPAI

SEQ ID NO: 471 - protein, 25 aa, Legionella pneumophila
EKEHQNQSSGQFDLFSLLEDKADEQ

SEQ ID NO: 472 - protein, 25 aa, Coxiella burnetii
EQRNRDMILGQHDLFGEEVKGIDED

SEQ ID NO: 473 - protein, 25 aa, Methylococcus capsulatus
EQQGAMSAAGQDDLFGGFTAESPAA

SEQ ID NO: 474 - protein, 25 aa, Pseudomonas aeruginosa
EQTARSHDSGHMDLFGGVFAEPEAD

SEQ ID NO: 475 - protein, 25 aa, Pseudomonas putida
EQAAHTADSGHVDLFGSMFDAADVD

SEQ ID NO: 476 - protein, 25 aa, Pseudomonas syringae
EQTARSHDSGHSDLFGGLFVEADAD

SEQ ID NO: 477 - protein, 25 aa, Pseudomonas fluorescens
EQTARTRDSGHADLFGGLFVEEDAD

SEQ ID NO: 478 - protein, 25 aa, Shewanella putrefaciens
DQHAKAEAIGQHDMFGLLNSDPEDS

SEQ ID NO: 479 - protein, 25 aa, Vibrio cholerae
SQHHQAEAFGQADMFGVLTDAPEEV

SEQ ID NO: 480 - protein, 25 aa, Pasteurella multocida
DQHAKDAAMGQADMFGVLTESHEDV

SEQ ID NO: 481 - protein, 25 aa, Haemophilus influenzae
DQHAKDEAMGQTDMFGVLTETHEDV

SEQ ID NO: 482 - protein, 25 aa, Haemophilus ducreyi
DQHSKMEALGQSDMFGVLTETPEQV

SEQ ID NO: 483 - protein, 25 aa, Actinobacillus actinomycetemcomitans
DQHAKDEALGQVDMFGVLTETNEEV

SEQ ID NO: 484 - protein, 25 aa, Buchnera sp.
KESFRIKSFKQDSLFGIFQNELNQV

SEQ ID NO: 485 - protein, 25 aa, Escherichia coli
DQHAKAEAIGQADMFGVLAEEPEQI

SEQ ID NO: 486 - protein, 25 aa, Salmonella typhi
DQHAKAEAIGQTDMFGVLAEEPEQI

SEQ ID NO: 487 - protein, 25 aa, Salmonella typhimurium
DQHAKAEAIGQTDMFGVLAEEPEQI

SEQ ID NO: 488 - protein, 25 aa, Yersinia pestis
DQHAKAEAIGQVDMFGVLADAPEQV

SEQ ID NO: 489 - protein, 25 aa, Desulfovibrio vulgaris
QKKLKERDSNQVSLFTMIKEEPKVC

SEQ ID NO: 490 - protein, 25 aa, Geobacter sulfurreducens
QKIQQEKESAQVSLFGAEEIVRTNG

SEQ ID NO: 491 - protein, 25 aa, Helicobacter pylori
KDKANEMMQGGNSLFGAMEGGIKEQ

SEQ ID NO: 492 - protein, 25 aa, Campylobacter jejuni
RKMAEVRKNAASSLFGEEELTSGVQ

SEQ ID NO: 493 - protein, 25 aa, Streptomyces coelicolor
VAVKRKEAEGQFDLFGGMGDEQSDE

SEQ ID NO: 494 - protein, 25 aa, Saccharopolyspora erythraea
IGLKRQQALGQFDLFGGGDDAGGEE

SEQ ID NO: 495 - protein, 25 aa, Thermobifida fusca
LSSKKQEAHGQFDLFGGGDEEDGGE

SEQ ID NO: 496 - protein, 25 aa, Mycobacterium avium
LGTKKAEAMGQFDLFGGDGGCTESV

SEQ ID NO: 497 - protein, 25 aa, Mycobacterium leprae
LGTKKAEAIGQFDLFGGTDGTDAVF

SEQ ID NO: 498 - protein, 25 aa, Mycobacterium smegmatis
LGTKKAEAMGQFDLFGGGEDTGTDA

SEQ ID NO: 499 - protein, 25 aa, Mycobacterium tuberculosis
LGTKKAEALGQFDLFGSNDDGTGTA

SEQ ID NO: 500 - protein, 25 aa, Corynebacterium diptheriae
TSTKKAADKGQFDLFAGLGADAEEV

SEQ ID NO: 501 - protein, 25 aa, Dehalococcoides ethenogenes
QREQKLKDSNQTTMFDLFGQQSPMP

SEQ ID NO: 502 - protein, 25 aa, Clostridium difficile
SMDRKKNVQGQISLFDAFGDSEEDS

SEQ ID NO: 503 - protein, 25 aa, Carboxydothermus hydrogenoformans
EFYSKKSNGVQLTLGDFLPEADRYN

SEQ ID NO: 504 - protein, 25 aa, Bacillus halodurans
AEQVKEFQENTGGLFQLSVEEPEYI

SEQ ID NO: 505 - protein, 25 aa, Bacillus stearothermophilus
IAIEHAQWVQALEAGGLSLKPKYAA

SEQ ID NO: 506 - protein, 25 aa, Bacillus subtilis
HAELFAADDDQMGLFLDESFSIKPK

SEQ ID NO: 507 - protein, 25 aa, Staphylococcus aureus
VLDGDLNIEQDGFLFDILTPKQMYE

SEQ ID NO: 508 - protein, 25 aa, Staphylococcus epidermidis
VLDLNSDVEQDEMLFDLLTPKQSYE

SEQ ID NO: 509 - protein, 25 aa, Bacillus anthracis
LKGALEYANLARDLGDAVPKSKYVQ

SEQ ID NO: 510 - protein, 25 aa, Listeria innocua
YISLLGEDSKGMNLFAEDDDFLKKM

SEQ ID NO: 511 - protein, 25 aa, Listeria monocytogenes
YISLLGEDSKGMNLFAEDDDFLKKM

SEQ ID NO: 512 - protein, 25 aa, Listeria monocytogenes
YISLLGEDSKGMNLFAEDDEFLKKM

SEQ ID NO: 513 - protein, 25 aa, Enterococcus faecalis
NIQSILLSGGSMDLLETLPKEEEIA

SEQ ID NO: 514 - protein, 25 aa, Enterococcus faecium
KIQNIVYSGGSLDLLGIMALKEEEV

SEQ ID NO: 515 - protein, 25 aa, Lactococcus lactis
ADHANLLNYYSDDIFMASSGGGFAY

SEQ ID NO: 516 - protein, 25 aa, Streptococcus equi
LEGLLTFVNELGSLFADSSFSWVET

SEQ ID NO: 517 - protein, 25 aa, Streptococcus pyogenes
LDGLLVFVNELGSLFSDSSFSWVDT

SEQ ID NO: 518 - protein, 25 aa, Streptococcus mutans
LEHLFTFVNELGSLFADSSYNWIEA

SEQ ID NO: 519 - protein, 25 aa, Streptococcus pneumoniae
LANLFEFVKELGSLFGDAIYSWQES

SEQ ID NO: 520 - protein, 25 aa, Ureaplasma urealyticum
EKTGLNGHFFDLNLVGLDYAKDMSV

SEQ ID NO: 521 - protein, 25 aa, Mycoplasma genitalium
NDAKDFWIKSDHLLFTRMPLEKKDS

SEQ ID NO: 522 - protein, 25 aa, Mycoplasma pneumoniae
NLAKSFWVQSNHELFPKIPLDQPPV

SEQ ID NO: 523 - protein, 25 aa, Mycoplasma pulmonis
LAKVQGDDIDISNFFQLEFSKNSSR

SEQ ID NO: 524 - protein, 25 aa, Clostridium acetobutylicum
SGQRKKNLKGQMNLFTDFVQDDYEE

SEQ ID NO: 525 - protein, 25 aa, Rhodopseudomonas palustris
WAVRRLPDDVPLPLFEAASAREQED

SEQ ID NO: 526 - protein, 25 aa, Mesorhizobium loti
RALGAKSAAEKLPLFDQPALRLREL

SEQ ID NO: 527 - protein, 25 aa, Brucella suis
WAVRRLPNDETLPLPRAAAASELAQ

SEQ ID NO: 528 - protein, 25 aa, Sinorhizobium meliloti
KALDEQSAVERLPLFEGAGSDDLQI

SEQ ID NO: 529 - protein, 25 aa, Sinorhizobium meliloti
LWAIKALRDEPLPLFTAAADREARA

SEQ ID NO: 530 - protein, 25 aa, Agrobacterium tumefaciens
LWAIKALRDEPLPLFAAAAIRENAV

SEQ ID NO: 531 - protein, 25 aa, Agrobacterium tumefaciens
LWAIKALRDEPLPLFAAAAEREATA

SEQ ID NO: 532 - protein, 25 aa, Agrobacterium tumefaciens
LWAIKALRDEPLPLFAAAAEREMAA

SEQ ID NO: 533 - protein, 25 aa, Caulobacter crescentus
GLKGEHKAPVQAPLLAGLPLFEERV

SEQ ID NO: 534 - protein, 25 aa, Rhodobacter capsulatus
WAVRAIRAPKPLPLFANPLDGEGGI

SEQ ID NO: 535 - protein, 25 aa, Sphingomonas aromaticivorans
LWDVRRTPPTQLPLFAFANAPELGQ

SEQ ID NO: 536 - protein, 24 aa, Bordetella bronchiseptica
AWQAAASAQSRDLLREAVIVETET

SEQ ID NO: 537 - protein, 25 aa, Bordetella parapertussis
ASWQAAASAQSRDLLREAVIVETET

SEQ ID NO: 538 - protein, 25 aa, Bordetella pertussis
ASWQAAASAQSRDLLREAVIVETET

SEQ ID NO: 539 - protein, 25 aa, Burkholderia pseudomallei
ALWQAVAAAPERGLLAAAPIDEAVR

SEQ ID NO: 540 - protein, 25 aa, Burkholderia cepacia
RWWAVTAQHAVPRLLRDAPIAEAAL

SEQ ID NO: 541 - protein, 25 aa, Ralstonia metallidurans
HARGAAVQTQHRDLLHDAPPQEHAL

SEQ ID NO: 542 - protein, 25 aa, Acidothiobacillus ferrooxidans
RHQALWAVQGSLPLPTALPMPVVPE

SEQ ID NO: 543 - protein, 25 aa, Methylococcus capsulatus
AFWEAAGVEAPTPLYAEPQFAEAEP

SEQ ID NO: 544 - protein, 25 aa, Pseudomonas aeruginosa
ARWAVASVEPQLPLFAEGTAIEEST

SEQ ID NO: 545 - protein, 25 aa, Pseudomonas putida
ARWQVAAVQPQLPLFADVQALPEEP

SEQ ID NO: 546 - protein, 25 aa, Pseudomonas syringae
ARWEVAGVEAQRPLFDDVTSEEVQV

SEQ ID NO: 547 - protein, 25 aa, Pseudomonas fluorescens
ARWEVAGVQKQLGLFAGLPSQEEPD

SEQ ID NO: 548 - protein, 25 aa, Mycobacterium avium
AGAAATQRPDRLPGVGSSSHIPALP

SEQ ID NO: 549 - protein, 18 aa, Mycobacterium leprae
RANRLPGVGGSSHIPVLP

SEQ ID NO: 550 - protein, 25 aa, Mycobacterium smegmatis
AGAAATQRPDRLPGVGSSTHIPPLP

SEQ ID NO: 551 - protein, 25 aa, Mycobacterium tuberculosis
AGAAATGRPDRLPGVGSSSHIPALP

SEQ ID NO: 552 - protein, 25 aa, Corynebacterium diptheriae
AGAAATEKAAMLPGLSMVSAPSLPG

SEQ ID NO: 553 - protein, 15 aa, Thermotoga maritima
GVLGDLPETEQFTLF

SEQ ID NO: 554 - protein, 19 aa, Desulfitobacterium hafniense
DCLKGIPESDQISFFDLIS

SEQ ID NO: 555 - protein, 15 aa, Clostridium difficile
GSLENMSERNQLSLF

SEQ ID NO: 556 - protein, 16 aa, Carboxydothermus hydrogenoformans
GCLKGLAPTSQLVLFA

SEQ ID NO: 557 - protein, 15 aa, Bacillus halodurans
GCLEGLPESNQLSLF

SEQ ID NO: 558 - protein, 15 aa, Bacillus stearothermophilus
GCLDSLPDHNQLSLF

SEQ ID NO: 559 - protein, 15 aa, Bacillus subtilis
GCLESLPDQNQLSLF

SEQ ID NO: 560 - protein, 17 aa, Staphylococcus aureus
GSLPNLPDKAQLSIFDM

SEQ ID NO: 561 - protein, 17 aa, Staphylococcus epidermidis
GSLPDLPDKAQLSIFDM

SEQ ID NO: 562 - protein, 15 aa, Bacillus anthracis
GCLGDLPDQNQLSLF

SEQ ID NO: 563 - protein, 15 aa, Listeria innocua
GCLEGLPDQNQLSLF

SEQ ID NO: 564 - protein, 15 aa, Listeria monocytogenes
GCLEGLPDQNQLSLF

SEQ ID NO: 565 - protein, 15 aa, Listeria monocytogenes
GCLEGLPDQNQLSLF

SEQ ID NO: 566 - protein, 18 aa, Enterococcus faecalis
GVLKDLPDENQLSLFDML

SEQ ID NO: 567 - protein, 15 aa, Enterococcus faecium
GVLKDLPDENQLSLF

SEQ ID NO: 568 - protein, 19 aa, Lactococcus lactis
GVLEGMPDDNQLSLFDDFF

SEQ ID NO: 569 - protein, 19 aa, Streptococcus equi
GILGNMPDDNQLSLFDDFF

SEQ ID NO: 570 - protein, 19 aa, Streptococcus pyogenes
GILGNMPEDNQLSLFDDFF

SEQ ID NO: 571 - protein, 19 aa, Streptococcus mutans
GILGSMPEDNQLSLFDDFF

SEQ ID NO: 572 - protein, 19 aa, Streptococcus thermophilus
GILGNMPEDNQLSLFDDFF

SEQ ID NO: 573 - protein, 19 aa, Streptococcus pneumoniae
GILGNMPEDNQLSLFDELF

SEQ ID NO: 574 - protein, 15 aa, Ureaplasma urealyticum
GVLDHLSETEQLTLF

SEQ ID NO: 575 - protein, 16 aa, Mycoplasma genitalium
QLFDEFEHQDDHKLFN

SEQ ID NO: 576 - protein, 15 aa, Mycoplasma pneumoniae
LLDEFREQDNQKKLF

SEQ ID NO: 577 - protein, 15 aa, Mycoplasma pulmonis
GIFEQIPETNQIFLI

SEQ ID NO: 578 - protein, 18 aa, Clostridium acetobutylicum
GCLKGLPESDQLSFFDAI

SEQ ID NO: 579 - protein, 25 aa, Acidothiobacillus ferrooxidans
PVSDTAFAGWQLSLFQGFLANTDDQ

SEQ ID NO: 580 - protein, 14 aa, Buchnera aphidicola
MLLFKILQSKFKKD

SEQ ID NO: 581 - protein, 25 aa, Escherichia coli
EKLDVIKDSPQMSLFEIIESPAKKD

SEQ ID NO: 582 - DNA, 30 bp, Artificial Sequence
  synthetic oligonucleotide primer
tggctggaattcaaatttaccgtagaacgt

SEQ ID NO: 583 - DNA, 30 bp, Artificial Sequence
  synthetic oligonucleotide primer
agtccagaattcttacagtctcattggcat

SEQ ID NO: 584 - DNA, 32 bp, Artificial Sequence
  synthetic oligonucleotide primer
tttgatgaattcaaaagcgacgttgaatacgc

SEQ ID NO: 585 - DNA, 32 bp, Artificial Sequence
  synthetic oligonucleotide primer
gctttggaattcgtgtcatatcaaacgttatg

SEQ ID NO: 586 - DNA, 40 bp, Artificial Sequence
  synthetic oligonucleotide primer
gactttgaattctcgagttaaccacgttctgtcgggtgca

SEQ ID NO: 587 - DNA, 32 bp, Artificial Sequence; identical to SEQ ID NO: 584
  synthetic oligonucleotide primer

SEQ ID NO: 588 - DNA, 40 bp, Artificial Sequence
  synthetic oligonucleotide primer
gactttgaattctcgagttacataacgtttgataagtcac

SEQ ID NO: 589 - DNA, 29 bp, Artificial Sequence
  synthetic oligonucleotide primer
gtcaggccgataaaaagggcgtgctggcc

SEQ ID NO: 590 - DNA, 29 bp, Artificial Sequence
  synthetic oligonucleotide primer
gccagcacgccctttttatcggcctgacc

SEQ ID NO: 591 - DNA, 39 bp, Artificial Sequence
  synthetic oligonucleotide primer
gaagctatcggtcctgccgatatgccaggcgtgctggcc

SEQ ID NO: 592 - DNA, 39 bp, Artificial Sequence
  synthetic oligonucleotide primer
ggccagcacgcctggcatatcggcaccaccgatagcttc

SEQ ID NO: 593 - DNA, 35 bp, Artificial Sequence
  synthetic oligonucleotide primer
ggaaagaattcggtccggcggcagatcaacacgcg

SEQ ID NO: 594 - DNA, 45 bp, Artificial Sequence
  synthetic oligonucleotide primer
gatcaactcgagaggacctccagctcccggctcttcggccagcac

SEQ ID NO: 595 - DNA, 43 bp, Artificial Sequence
  synthetic oligonucleotide primer
tctcaaagaattcgcagcgggtgcgagtcagggagtcgcgcag

SEQ ID NO: 596 - DNA, 36 bp, Artificial Sequence
  synthetic oligonucleotide primer
aatccactcgaggcctccaccgatagcttccgcttt

SEQ ID NO: 597 - DNA, 40 bp, Artificial Sequence
  synthetic oligonucleotide primer
tctcaaagaattcgcgggtgcgagtcagggagtcgcgcag

SEQ ID NO: 598 - DNA, 33 bp, Artificial Sequence
  synthetic oligonucleotide primer
aatccactcgagtcccggtgcgttgtcatcgaa

SEQ ID NO: 599 - DNA, 40 bp, Artificial Sequence
  synthetic oligonucleotide primer
tctcaaagaattcgcgggtgcgccgcaaatggaaagacaa

SEQ ID NO: 600 - DNA, 39 bp, Artificial Sequence
  synthetic oligonucleotide primer
aatccactcgagtccagctcctaatcccagcaccagttg

SEQ ID NO: 601 - DNA, 26 bp, Artificial Sequence
  synthetic oligonucleotide primer
tctcaaagccgccgctacgcaagtgg

SEQ ID NO: 602 - DNA, 40 bp, Artificial Sequence
  synthetic oligonucleotide primer
aatccactcgagtccagctcctggtactgacagcaaagac

SEQ ID NO: 603 - DNA, 30 bp, Artificial Sequence
  synthetic oligonucleotide primer
gggaattccatatgttcgaggcgcgcctgg

SEQ ID NO: 604 - DNA, 35 bp, Artificial Sequence
  synthetic oligonucleotide primer
cgaagctttgcggccgccagtctcattggcatgac

SEQ ID NO: 605 - DNA, 30 bp, Artificial Sequence
  synthetic oligonucleotide primer
gggaattcccatatgtatcgtaaagatttg

SEQ ID NO: 606 - DNA, 39 bp, Artificial Sequence
  synthetic oligonucleotide primer
ccgctcgagtgcggccgcggggttaatgattttttgaat

SEQ ID NO: 607 - DNA, 32 bp, Artificial Sequence
  synthetic oligonucleotide primer
gggaattccatatgaaaaactccaaccgcctt

SEQ ID NO: 608 - DNA, 39 bp, Artificial Sequence
  synthetic oligonucleotide primer
ccgctcgagtgcggccgctggcgttttctttttggataa

SEQ ID NO: 609 - DNA, 26 bp, Artificial Sequence
  synthetic oligonucleotide primer
gggaattccatatggaaatcagtgtt

SEQ ID NO: 610 - DNA, 35 bp, Artificial Sequence
  synthetic oligonucleotide primer
cgaagctttgcggccgcttatagtgtgattggcat

SEQ ID NO: 611 - DNA, 27 bp, Artificial Sequence
  synthetic oligonucleotide primer
ggcatacatatgaaatttaccgtagaa

SEQ ID NO: 612 - DNA, 35 bp, Artificial Sequence
  synthetic oligonucleotide primer
ctcgagtgcggccgcttacagtcttattggcatga

SEQ ID NO: 613 - DNA, 30 bp, Artificial Sequence
  synthetic oligonucleotide primer
ctggaattctatcgtaaagatttggaccat

SEQ ID NO: 614 - DNA, 39 bp, Artificial Sequence; identical to SEQ ID NO: 606
  synthetic oligonucleotide primer

SEQ ID NO: 615 - DNA, 30 bp, Artificial Sequence
  synthetic oligonucleotide primer
ctggaattcaaaaactccaaccgccttatt

SEQ ID NO: 616 - DNA, 39 bp, Artificial Sequence; identical to SEQ ID NO: 608
  synthetic oligonucleotide primer

SEQ ID NO: 617 - DNA, 37 bp, Artificial Sequence
  synthetic oligonucleotide primer
cactaaagggcggccgcatgaaagcgttaacggccag

SEQ ID NO: 618 - DNA, 30 bp, Artificial Sequence
  synthetic oligonucleotide primer
cgcctcgagatgcaagttttagcgttaaaa

SEQ ID NO: 619 - DNA, 36 bp, Artificial Sequence
  synthetic oligonucleotide primer
cgaggagcctcgagtcataacaattccacgcttttg

SEQ ID NO: 620 - DNA, 34 bp, Artificial Sequence
  synthetic oligonucleotide primer
gccaggctatgagtgcggctgccagtcgacaaac

SEQ ID NO: 621 - DNA, 34 bp, Artificial Sequence
  synthetic oligonucleotide primer
gtttgtcgactggcagccgcactcatagcctggc

SEQ ID NO: 622 - protein, 5 aa, Artificial Sequence
  synthetic peptide
QLSLF

SEQ ID NO: 623 - protein, 5 aa, Artificial Sequence
  synthetic peptide
QLSMF

SEQ ID NO: 624 - protein, 5 aa, Artificial Sequence
  synthetic peptide
QLDMF

SEQ ID NO: 625 - protein, 5 aa, Artificial Sequence
  synthetic peptide
QLDLF

SEQ ID NO: 626 - protein, 5 aa, Artificial Sequence
  synthetic peptide
HLSLF

SEQ ID NO: 627 - protein, 5 aa, Artificial Sequence
  synthetic peptide
HLSMF

SEQ ID NO: 628 - protein, 5 aa, Artificial Sequence
  synthetic peptide
HLDMF

SEQ ID NO: 629 - protein, 5 aa, Artificial Sequence
  synthetic peptide
HLDLF

SEQ ID NO: 630 - protein, 5 aa, Artificial Sequence
  synthetic peptide
QLNLF

SEQ ID NO: 631 - protein, 5 aa, Escherichia coli
QADMF

SEQ ID NO: 632 - protein, 5 aa, Artificial Sequence
  synthetic peptide
QADKK

SEQ ID NO: 633 - protein, 5 aa, Escherichia coli
PADMP

SEQ ID NO: 634 - protein, 24 aa, Escherichia coli
AADQHAKAEAIGQADMFGVLAEEP

SEQ ID NO: 635 - protein, 24 aa, Escherichia coli
AALMNSLGADLKAADQHAKAEAIG

SEQ ID NO: 636 - protein, 5 aa, Escherichia coli
QLGLF

SEQ ID NO: 637 - protein, 15 aa, Escherichia coli
SQGVAQLNLFDDNAP

SEQ ID NO: 638 - protein, 17 aa, Escherichia coli
AAATQVDGTQMSLLSVP

SEQ ID NO: 639 - protein, 11 aa, Escherichia coli
PQMERQLVLGL

SEQ ID NO: 640 - protein, 9 aa, Escherichia coli
IGQADMFGV

SEQ ID NO: 641 - protein, 9 aa, Artificial Sequence
  synthetic peptide
IGQLDMFGV

SEQ ID NO: 642 - protein, 9 aa, Artificial Sequence
  synthetic peptide
IGQASMFGV

SEQ ID NO: 643 - protein, 9 aa, Artificial Sequence
  synthetic peptide
IGQADAFGV

SEQ ID NO: 644 - protein, 9 aa, Escherichia coli
IGQADMAGV

SEQ ID NO: 645 - protein, 9 aa, Artificial Sequence
  synthetic peptide
IGQAVMFGV

SEQ ID NO: 646 - protein, 9 aa, Artificial Sequence
  synthetic peptide
IGPADMFGV

SEQ ID NO: 647 - protein, 9 aa, Artificial Sequence
  synthetic peptide
IGKADMFGV

SEQ ID NO: 648 - protein, 9 aa, Artificial Sequence
  synthetic peptide
IGQADKFGV

SEQ ID NO: 649 - protein, 9 aa, Artificial Sequence
  synthetic peptide
IGQADMKGV

SEQ ID NO: 650 - protein, 9 aa, Artificial Sequence
  synthetic peptide
IGQAAMFGV

SEQ ID NO: 651 - protein, 9 aa, Artificial Sequence
  synthetic peptide
IGAADMFGV

SEQ ID NO: 652 - protein, 9 aa, Artificial Sequence
  synthetic peptide
IGQLSLFGV

SEQ ID NO: 653 - protein, 9 aa, Artificial Sequence
  synthetic peptide
IGQLDLFGV

SEQ ID NO: 654 - protein, 10 aa, Artificial Sequence
  synthetic peptide
IGQAMSLFGV

SEQ ID NO: 655 - protein, 10 aa, Artificial Sequence
  synthetic peptide
IGQLVLGLGV

SEQ ID NO: 656 - protein, 10 aa, Artificial Sequence
  synthetic peptide
IGQLSLPLGV

SEQ ID NO: 657 - protein, 8 aa, Artificial Sequence
  synthetic peptide
IGLNLFGV

SEQ ID NO: 658 - protein, 9 aa, Artificial Sequence
  synthetic peptide
IGQMSLLGV

SEQ ID NO: 659 - protein, 9 aa, Artificial Sequence
  synthetic peptide
IGQLGLFGV

SEQ ID NO: 660 - protein, 10 aa, Escherichia coli
PAQLSLPLYL

SEQ ID NO: 661 - protein, 9 aa, Escherichia coli
EAQLDLFDS

SEQ ID NO: 662 - protein, 5 aa, Artificial Sequence
  synthetic peptide
QLDLF

SEQ ID NO: 663 - protein, 9 aa, Artificial Sequence
  synthetic peptide
IGQLDLFGV

SEQ ID NO: 664 - protein, 180 aa, Artificial Sequence
  truncated E. coli tau protein
HHAYLFSGTRGVGKTSIARLLAKGLFVDLIEIDAARDLLDNVQYAPARGRFKVYLIDEVHMLSRHSFNALLKTLEEPPEHVKFLLATTDPQKLPVTILSRCLQFHLKALDVEQIRHQLEHILNEEHIAHEPRALQLLARAAEGSLRDALSLTDQAIASGDGQVSTQAVSAMLGTLDDDQA

SEQ ID NO: 665 - protein, 175 aa, Artificial Sequence
  truncated E. coli delta′ protein
HHALLIQALPGMGDDALIYALSRYLHPDYYTLAPEREVTEKLNEHARLGGAKVVWVTDAALLTDAAANALLKTLEEPPAETWFFLATREPERLLATLRSRCRLHYLAPPPEQYAVTWLSREVTMSQDALLAALRLSAGSPGAALALFQGDNWQARETLCQALAYSVPSGDWYSLL

SEQ ID NO: 666 - protein, 196 aa, Artificial Sequence
  synthetic truncated E. coli delta protein
RAAYLLLGNDPLLLQESQDAVRQVAAAQGFEEHHTFSIDPNTDWNAIFSLCQAMSLFASRQTLLLLLPENGPNAAINEQLLTLTGLLHDDLLLIVRGNKLSKAQENAAWFTALANRSVQVTCQTPEQAQLPRWVAARAKQLNLELDDAANQVLCYCYEGNLLALAQALERLSLLWPDGKLTLPRVEQAVNDAAHFT

SEQ ID NO: 667 - protein, 185 aa, Artificial Sequence
  synthetic truncated R. prowazekii delta protein
IRALLLYGPDKGYIEKICTYLIKNLNMLQSSIEYEDLNILSLDILLNSPNFFGQKELIKVRSIGNSLDKNLKTILSSDYINFPVFIGEDMNSSGSVKKFFETEEYLAVVACYHDDEAKIERIILGKLAKTNKVISKEAITYLKTHLKGDHALICSEINKLIFFAHDVHEITLNHVLEVISSEITA

SEQ ID NO: 668 - protein, 208 aa, Artificial Sequence
  synthetic truncated H. pylori delta protein
PKAVFLYGEFDFFIHYYIQTISALFKGNNPDTETSLFYASDYEKSQIATLLEQDSLFGGSSLVILKLDFALHKKFKENDINPFLKALERPSHNRLIIGLYNAKSDTTKYKYTSEIIVKFFQKSPLKDEAICVRFFTPKAWESLKFLQERANFLHLDISGHLLNALFEINNEDLSVSFNDLDKLAVLNAPITLEDIQELSSNAGDMDLQ

SEQ ID NO: 669 - protein, 193 aa, Artificial Sequence
  synthetic truncated M. tuberculosis delta protein
MHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRG

SEQ ID NO: 670 - protein, 203 aa, Artificial Sequence
  synthetic truncated B. subtilis delta protein
HPVYCLYGKETYLLQETVSRIRQTVVDQETKDFNLSVFDLEEDPLDQAIADAETFPFMGERRLVIVKNPYFLTGEKKKEKIEHNVSALESYIQSPAPYTVFVLLAPYEKLDERKKLTKALKKHAFMMEAKELNAKETTDFTVNLAKTEQKTIGTEAAEHLVLLVNGHLSSIFQEIQKLCTFIGDREEITLDDVKMLVARSLEQ

SEQ ID NO: 671 - protein, 180 aa, Artificial Sequence
  synthetic truncated M. pneumoniae delta protein
MTVVYGADIGLIHQQLNQLLNPAACKQVWFQDVNKLYDVVLNQNLFAEDTKPILIHNCSFLEKNNLTKAELHCLKTLKDTDVVVTIYSDSPANALINDRAITKYACKPVTAKTIHQVISKAAKTLKLNLNPDLIDHLATILPFNLGVIEQELRKLTLLSPAELQDKKMLEAVLCDYQTSQ

SEQ ID NO: 672 - protein, 174 aa, Artificial Sequence
  synthetic truncated B. burgdoferi delta protein
QAVYLLLGNEQGLKEAYLKELLIKMDAFKSEVSVTKIFLSELSAVGFAEKLFSNSFFSKKEIFIVYESELLKAGKDLELVCNSILKSNNKTVIFVSNSNTCNIDFKNKLKFIKRNFFNLNIKITDSAINLMLLMLNSDTKILKFYIDSFALFAKNNTIEEEDIASWISFIRFEN

SEQ ID NO: 673 - protein, 193 aa, Artificial Sequence
  synthetic truncated T. pallidum delta protein
MSVWLFTGPEIGERDSAVQEVCARAQAQGTVDVHRLYAHETPVADLVDLLRTRALFADAVCVVLYNAEVIKKCDEVHVLTEWIKDGGSRADVFLVLISDSVSIHKRIEQNISPVHKRVFWELFENKKHAWVQRFFFQHEMRIEQEAIESLLELVENNTRALKTVCTQLSLFFEKGRRITAHDISSLLVHTKEE

SEQ ID NO: 674 - protein, 201 aa, Artificial Sequence
  synthetic truncated Synechocystis sp. delta protein
MPVYFYWGEDQFTLHQAVKQLQKRCLDPQWEAFNFEKIPGEQADATQRGLEQALTPPFGSGDRLVWVVDSTLGQSCDDGLLARLQKSLPAIPTDCHLLFTSSKKLDRRLKSTKYLEGNATIREFALISPWNVDALIHQIQAIAQDLQLPLATETEGFLAEALGNDTRLIWNELGKLKLYSESQTGPLTVAQVEQLVNTSTQ

SEQ ID NO: 675 - protein, 189 aa, Artificial Sequence
  synthetic truncated C. pneumoniae delta protein
VPAIALIGSALEDDKDALIELLVSESFKELGGQGLMPATLMSWTETFALFQEHETLGIIHAEKFPLATKEFLSRYARNPQPHLTILIFTTKQECFRELSKALPSALSLSLFGEWPADRQKRIIRLLLQRAERVGISCSQSLASLFLRALASTSLPDILSEFDKLLCSVGKKTSLDHSDIKELVVKKEKA

SEQ ID NO: 676 - protein, 181 aa, Artificial Sequence
  synthetic truncated D. radiodurans delta protein
MPVLAFTGNRFLADETLRDTLSARGLNARDLPRFSGEDVSAETLGPHLAPSLFGDGGVVVDFEGLKPDKALLELLSSAPVTVAVLDEAPPATRLKLYQKAGEVIPSAAPSKPGDVTGWVVTRAKKMGLRLERDAASYLAEVFGADLAGIAGELNKLELLGGALNRERVQGIVGRDPPGDSF

SEQ ID NO: 677 - protein, 179 aa, Artificial Sequence
  synthetic truncated T. maritima delta protein
MPVTFLTGTAETQKEELIKKLLKDGNVEYIRIHPEDPDKIDFIRSLLRTKTIFSNKTIIDIVNFDEWKAQEQKRLVELLKNVPEDVHIFIRSQKTGGKGVALELPKPWETDKWLEWIEKRFRENGLLIDKDALQLFFSKVGTNDLIIEREIEKLKAYSEDRKITVEDVEEVVFTYQTPG

SEQ ID NO: 678 - protein, 198 aa, Artificial Sequence
  synthetic truncated A. aeolicus delta protein
ERVFVLHGEEQYLIRTFLSKLKEKYGENYTVLWGDEISEEEFYTALSETSIFGGSKEKAVVIYNFGDFLKKLGRKKKEKERLIKVLRNVKSNYVFIVYDAKLQKQELSSEPLKSVASFGGIVVANRLSKERIKQLVLKKFKEKGINVENDALEYLLQLTGYNLMELKLEVEKLIDYASEKKILTLDEVKRVAFSVSEN

(19) United States
(12) Patent Application Publication
(10) Pub. No.: US 2004/0132121 A1
(43) Pub. Date: Jul. 8, 2004

(54) Method of identifying antibacterial compounds

(76) Inventors: Brian Paul Dalrymple, Indooroopilly (AU); Kritaya Kongsuwan, Brookfield (AU); Gene-Louise Wilfiels, Chapel Hill (AU); Philip Anthony Jennings, Killara (AU); Gregory William Komp, Indooroopilly (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/416,249

(22) PCT Filed: Nov. 8, 2001
(86) PCT No.: PCT/AU01/01436

(30) Foreign Application Priority Data
Nov. 8, 2000 (AU) PR 1320
Feb. 6, 2001 (AU) PR 2919

(51) Int. Cl. G01N 33/554; G01N 33/569

(52) U.S. Cl. 435/7.32

(57) ABSTRACT

The present invention relates to peptides having eubacterial b protein-binding properties and the surface of b protein with which said peptides and other proteins interact. The invention provides in vitro and in vivo assays for identifying compounds that modulate the interaction between b protein and proteins that interact therewith, and a method of controlling eubacterial infestation by modulating this interaction. The disclosed peptides can be used as templates for the design or selection of compounds that modulate the foregoing interaction.

### TECHNICAL FIELD

The invention described herein in general relates to bacterial replication. More specifically, the invention relates to compounds useful as inhibitors of bacterial replication. In particular, the invention relates to a method of identifying compounds useful as inhibitors of bacterial replication, the compounds so identified, and use of the compounds as antibacterial agents in the treatment or prevention of disease in humans, animals and plants.

### BACKGROUND ART

Diseases due to bacterial infections of humans continue to cause suffering and economic loss despite the availability of antibacterial agents. Bacterial diseases of animals similarly cause suffering to afflicted animals and economic loss in instances where the diseased animals are of agricultural value. Although hundreds of different antibacterial compounds are known, there is a continual need for alternative, more efficacious compounds. This is particularly so since bacterial strains that are resistant to existing antibacterial agents have emerged. In addition to identifying new antibacterial agents, it is desirable to identify classes of compounds whose modes of action are different to known classes of compounds. By identifying a class of compounds with a new mode of antibacterial activity, the armoury of agents that can be used against bacterial disease is greatly enlarged.

Each form of life must duplicate its genetic material to propagate. Consequently, a potentially useful mode of action for antibacterial agents would be by interference with the duplication, or replication, of the target bacterium's genetic material. The replication of bacterial genetic material (DNA) is reasonably well understood and numerous proteins are known to be involved: see the review by A. Kornberg et al., in DNA Replication, Second Edition, pp. 165-194, W. H. Freeman & Co., New York, 1992. During replication, most of these proteins are organised into a complex multifunctional machine referred to as “the replisome”.

In eubacteria, the central enzyme of the replisome is DNA Polymerase III holoenzyme. In Escherichia coli (E. coli) this enzyme contains 10 different subunits, whilst in most other bacteria only seven subunits have been identified. In E. coli, and probably in most other eubacteria, the DnaE orthologue (α subunit) is the main replicative polymerase, but in many gram positive organisms a distinct, but related enzyme, PolC is proposed to be the main replicative enzyme replacing DnaE in the replication machine. The processivity of the replisome is conferred by the β subunit of DNA Polymerase III, which forms a clamp around the DNA. The β subunit is loaded as a homodimer onto DNA by a clamp loader complex comprising single subunits of δ and δ′ and four subunits of τ/γ. All eubacteria studied to date contain genes encoding orthologues of the DnaE, β, δ, δ′ and τ/γ subunits of DNA Polymerase III and in E. coli these subunits have been shown to be essential for DNA replication.

The β dimer, which encircles the DNA, but does not actually bind to it, confers processivity on DNA Polymerase III by maintaining the close proximity of the DnaE or PolC subunits to the DNA. It has recently been proposed that β may also act as an effector that increases the intrinsic rate of DNA synthesis (see Klemperer et al., J. Biol. Chem. (2000) 275: 26136-26143). In addition to DnaE, three other DNA polymerases present in E. coli (all of which are regulated by the LexA repressor protein) appear to interact with β. PolB (PolII) is involved in DNA repair and the addition of β and the clamp loader complex leads to an increase in enzyme processivity in in vitro assays (Hughes et al., J. Biol. Chem. (1991) 267: 11431-11438). The addition of β and the clamp loader complex to DNA Polymerase IV (DinB) does not increase the processivity of DNA synthesis, rather it dramatically increases the efficiency of synthesis (Tang et al., Nature (2000) 404:1614-1018). The β subunit appears to play a similar role in the activity of DNA Polymerase V, the UmuD′2UmuC complex (Tang et al., 2000).

While the site on β to which the δ and α subunits of E. coli DNA polymerase III bind has been studied in some detail, the nature of the site(s) on δ, α and the other proteins that interact with β is not known. Experimental evidence shows that at least some β-binding proteins can interact productively with β proteins from heterologous species. For example, Staphylococcus aureus, Streptococcus pyogenes and Bacillus subtilis PolC subunits can use E. coli β as their processivity subunit (Low et al., J. Biol. Chem. (1976) 251: 1311-1325); Bruck and O'Donnell, J. Biol. Chem. (2000) 275: 28971-28983); Klemperer et al., 2000). In contrast, E. coli DnaE cannot use β from the other species (Klemperer et al., 2000), the Helicobacter pylori δ subunit does not bind to E. coli β, E. coli clamp loading complex cannot load S. aureus β (Klemperer et al., 2000) and the Streptococcus pyogenes clamp loading complex cannot load E. coli β P (Bruck and O'Donnell, 2000). These findings indicate that there is a degree of specificity in the interaction of other replisome proteins with β.

For an antibacterial agent to be of use, it must have limited activity against at least eukaryotes so that it does not have an adverse effect on the infected host, human or animal. In some circumstances, it is desirable that the antibacterial has activity against a limited range of bacteria such as a particular genus. The finding that there is specificity in the interaction of eubacterial replisome proteins with β protein raises the possibility that the interaction can be exploited as a mode of action of antibacterial agents with selectivity for members of the eubacteria.

### SUMMARY OF THE INVENTION

The primary object of the invention is to provide a method of identifying new antibacterial agents with selectivity for members of the eubacteria. Other objects of the invention will become apparent from a reading of the following summary and detailed description.

In a first embodiment, the invention provides a molecule comprising a surface analogous to the surface of the domain of eubacterial β protein contacted by proteins that interact with β protein, wherein said surface is defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:

X¹⁷⁰is any one of V, I, A, T, S or E;

X¹⁷² is any one of T, S or I;

X¹⁷⁵ is any one of H, Y, F, K, I, Q or R;

X¹⁷⁷ is any one of L, M, I, F, V or A;

X²⁴¹ is any one of F, Y or L;

X²⁴² is any one of P, L or I;

X²⁴⁷ is any one of V, I, A, F, L or M;

X³⁴⁶ is any one of S, P, A, Y or K;

X³⁶⁰ is any one of I, L or V; and

X³⁶² is anyone of M, L, V, S, T or R.

In a second embodiment, the invention provides a method of identifying a modulator of the interaction between a eubacterial β protein and proteins that interact therewith, the method comprising the steps of:

(a) forming a reaction mixture comprising:

(i) a ligand for eubacterial β protein that binds to at least part of the surface of β protein as defined in the first embodiment;

(ii) an interaction partner for said ligand; and

(iii) a test compound;

(b) incubating said reaction mixture under conditions which in the absence of said test compound allows interaction between said ligand and said interaction partner; and

(c) assessing the effect of said test compound on said interaction between said ligand and said interaction partner.

In a third embodiment, the invention provides a method for the in vivo identification of a modulator of the interaction between a eubacterial β protein and proteins that interact therewith, the method comprising the steps of:

(a) modifying a host to express or contain:

(i) a ligand for eubacterial β protein that binds to at least part of the surface of β protein as defined in the first embodiment; and

(ii) an interaction partner for said ligand;

(b) administering a test compound to said host and incubating the host under conditions which in the absence of said test compound allows interaction between said ligand and said interaction partner; and

(c) assessing the effect of said test compound on said interaction between said ligand and said interaction partner.

In a fourth embodiment, the invention provides a method of selecting a modulator of the interaction between a eubacterial β protein and proteins that interact therewith, the method comprising the steps of:

(a) establishing a consensus sequence for peptides that bind to at least part of the surface of β protein as defined in the first embodiment;

(b) modelling the structure of at least a portion of said consensus sequence and searching compound databases for compounds having a similar structure; wherein said modelling is by:

(i) searching protein databases for occurrences of said consensus sequence or portion thereof, obtaining coordinates of residues of proteins comprising said consensus sequence or portion thereof, and superimposing said coordinates to produce a pharmacophore model; or

(ii) modelling or determining the structure of a peptide comprising said consensus sequence or a portion thereof when bound to β protein; and

(c) testing compounds identified in step (b) for their effect on said interaction.

In a fifth embodiment, the invention provides a method of reducing the effect of eubacterial infestation of a biological system, the method comprising delivering to a system infested with a eubacterial species a modulator of the interaction between eubacterial β protein and proteins that interact therewith.

In a sixth embodiment, the invention provides a template for the design of a compound that binds to at least part of the surface of β protein as defined in the first embodiment, said template comprising a peptide selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴is A or G; X⁵ is L; and, X⁶is L, I, V, C, F, Y, W or P.

The foregoing and other embodiments of the invention will be described in detail below in conjunction with the drawings briefly described hereafter.

### BRIEF DESCRIPTION OF THE DRAWINGS

FIG. 1 is a schematic of the organisation of the domains of the DnaE and PolC subunits of the eubacterial DNA Polymerase III holoenzyme.

FIG. 2 gives results of a yeast two-hybrid experiments with LexA-β-binding motif protein fusions.

FIG. 3 gives structural alignments of amino acid sequences of examples of eubacterial δ proteins with sequences of E. coli δ′ and γ/τ proteins. The sequences are designated as follows: tau/gamma, E. coli (Seq. ID No. 664); delta′, E. coli (Seq. ID No. 665); Ec, E. coli (Seq. ID No. 666); Rp, Rickettsia prowazekii (Seq. ID No. 667); Hp, Helicobacter pylori (Seq. ID No. 668); Mt, Mycobacterium tuberculosis (Seq. ID No. 669); B, Bacillus subtilis (Seq. ID No. 670); Mp, Mycoplasma pneumoniae (Seq. ID No. 671); Bb, Borrelia burgdorferi (Seq. ID No. 672); Tp, Treponema pallidum (Seq. ID No. 673); S, Synechocystis sp. (Seq. ID No. 674); Cp, Chlamydiophila pneumoniae (Seq. ID No. 675); Dr, Deinococcus radiodurans (Seq. ID No. 676); Tm, Thermotoga maritima (Seq. ID No. 677); and Aa, Aquifex aeolicus (Seq. ID No. 678).

FIG. 4 gives the results of in vitro expression and interaction of H. pylori DNA Polymerase III subunits.

FIG. 5 gives the results of experiments to test the interaction of H. pylori DNA Polymerase III subunits in yeast two-hybrid assays.

FIG. 6 gives results for the expression of β-galactosidase in yeast two-hybrid assays.

FIG. 7 is a structural model of E. coli δ protein, showing the β-binding region.

FIG. 8 gives the results of experiments to test the interaction of native and mutant E. coli δ subunits.

FIG. 9 is an analysis of the distribution of amino acids in the pentapeptide β-binding motif. A single peptide sequence with three or more matches to the motif Qxshh (were ‘x’ is any amino acid, ‘s’ is any small amino acid and ‘h’ is any hydrophobic amino acid) in the appropriate region of the protein from each member of the PolC (22 representatives included), PolB (15 representatives included), DnaE1 (72 representatives included), UmuC (20 representatives included), DinB1 (62 representatives included) and MutS1 (59 representatives included) families of proteins is included in the analysis. Percentage frequency is plotted for each amino acid at each position of the pentapeptide motif.

FIG. 10 gives the results of an experiment in which inhibition of growth of B. subtilis by tripeptide DLF was tested.

FIG. 11 shows the three dimensional structure of E. coli β. The location of the residues described in the first embodiment are indicated by dark space-filled atoms.

### DETAILED DESCRIPTION OF THE INVENTION

The one- and three-letter codes for amino acid residues in proteins and for nucleotides in DNA conform to the IUPAC-IUB standard described in Biochemical Journal 219, 345-373 (1984).

The term “ligand” is used herein in the sense that it is a compound that binds to another compound, such as a protein, or to a cell, by way of non-covalent bonds at a specific site of interaction. This meaning of the term is in accordance with its usage by, for example, B. Alberts et al. in Molecular Biology of the Cell (Garland Publishing, Inc, New York and London, 1983: seepage 127).

The term “interaction” is used herein to embrace the specific binding of one molecule to another molecule without limitation as to the strength of binding or the physical nature of the association.

The term “modulator” is used herein to denote a compound that either enhances or inhibits the interaction between β protein and a ligand therefor. Modulators are thus either agonists or antagonists of the interaction.

The present invention stems from the identification, in a broad range of species of eubacteria, of a peptide motif responsible for the binding of proteins involved in DNA replication and repair to the clamp protein, β. The identification of this motif has also allowed elucidation of the β protein domain responsible for the interaction with proteins that bind thereto. We teach herein the parameters for designing compounds that inhibit the interaction of proteins with β. We also teach how to develop simple reagents for facilitating the screening of compounds for inhibitory or stimulatory activity. In particular, the development of a wide range of simple and robust assay systems for high throughput screening of natural products or synthetic compounds for such activity. From an understanding of the structures of the participants of the various protein-protein interactions involving the β protein and its ligands, new antibacterial agents with selective activity against eubacteria can be designed and the activity—including inhibitory and stimulatory activity—of such compounds tested by methods to be described in detail below. In addition, compounds are described with inhibitory activity in binding assays and with in vivo antibacterial activity.

The present inventors have established that peptides having eubacterial β protein-binding properties comprise at least the dipeptide X¹X², wherein X¹ is L, M, I, or F, and X² is L, I, V, C, F, Y, W, P, D, A or G. Peptides advantageously comprise a tripeptide, a tetrapeptide, a pentapeptide or a hexapeptide. Preferred dipeptides are X¹F wherein X¹ is as defined above. Preferred tripeptides are X³X¹X² wherein X¹ and X² are as defined above and X³ is A, G, T, N, D, S, or P. Preferred tetrapeptides are X³X¹X²X⁴ wherein X¹, X² and X³ are as previously defined and X⁴ is A or G. Preferred pentapeptides are QX⁵X³X¹X² wherein X¹, X² and X³ are as above and X⁵ is L. Particularly preferred pentapeptides are QLxLxL. Preferred hexapeptides are QX⁵xX⁶X³X⁶ wherein x, X³ and X⁵ are as defined above and X⁶ is L, I, V, C, F, Y, W or P.

Particularly preferred specific pentapeptides are QLSLF (Seq. ID No. 622), QLSMF (Seq. ID No. 623), QLDMF (Seq. ID No. 624) and QLDLF (Seq. ID No. 625). For Pseudomonads, the pentapeptides HLSLF (Seq. ID No. 626), HLSMF (Seq. ID No. 627), HLDMF (Seq. ID No. 628) and HLDLF (Seq. ID No. 629) are advantageous. Particularly preferred tetrapeptides are X³LFX⁴, wherein X⁴ is either A or G. Particularly preferred tripeptides are SLF, SMF, DLF and DMF. Particularly preferred dipeptides are LF and MF. The examples below give further details of preferred peptides.

The peptides set out above have utility as:

(i) reagents for the assay of modulators of the interaction between β protein and any ligand therefor;

(ii) inhibitors per se of the interaction between β protein and any ligand therefor;

(iii) templates for the design of molecules that modulate the interaction between β protein and any ligand therefor; and

(iv) determining the surface of the binding domain on β protein with which ligands interact from which surface modulators of the interaction can also be designed.

Peptides according to the invention can be synthesised and/or modified (see discussion on mimetics below) by any of the methods known to those of skill in the art. Alternatively, peptides can be excised from larger polypeptides that include the desired peptide sequence. The larger polypeptide can be produced by recombinant DNA means, as can the peptide per se.

With regard to the first embodiment of the invention as defined above, the three dimensional structure of the binding surface of β is defined by the co-ordinates of the residues specified above in the tertiary structure of E. coli β as described by Kong et al. (see Cell (1992) 69: 425-437).

Molecules including surfaces according to the first embodiment have utility as:

(i) reagents for the assay of the interaction between β protein and any ligand therefor;

(ii) modulators per se of the interaction between β protein and any ligand therefor;

(iii) templates for the design of molecules that inhibit the interaction between β protein and any ligand therefor;

(iv) templates for modelling the structure of the of the binding domain on β protein from which structure modulators of the interaction can also be designed;

(v) direct target sites for covalent and non-covalent interactions with compounds; and

(vi) indirect target sites, wherein said site or part of the site is obscured by compounds covalently or non-covalently bound elsewhere on β or β-binding proteins, peptides or compounds.

Regarding the second embodiment, the ligand can be any entity that binds to the β protein at the surface or part of the surface defined in the first embodiment or a mimetic of these domains or surfaces of the β protein. The ligand can thus range from a simple organic molecule to a complex macromolecule, such as a protein. Typical protein ligands include, but are not limited to, δ, DnaE1, DnaE2, PolC, PolB2, UmuC, DinB1, DinB2, DinB3, MutS1, RepA, Duf72 and DnaA2, and fragments thereof that are responsible for the interaction with β protein. Ligands also include the peptides defined above and mimetics of the peptides derived from β-binding proteins fused in whole or in part to other proteins, such as LexA, GST or GFP, peptides derived from β-binding proteins fused to other proteins such as LexA, GST or GFP, peptides as defined above that bind to eubacterial β proteins, but derived from proteins that do not themselves bind to β. Ligands also include antibodies and related molecules, such as single chain antibodies, that bind in whole or in part at or near to the surface of β protein as defined above in the first embodiment of the invention.

In the context of the present invention, the term “mimetic” of a peptide includes a fragment of a protein, peptide or any chemical form that provides substituents in the appropriate positions to enable the binding of compounds, in whole or in part, to the binding site on β protein in the manner of the peptides identified above. Those of skill in the art will be aware of the approaches that can be for the design of peptide mimetics when there is little or no secondary and tertiary structural information on the peptide. These approaches are described, for example in an article by Kirshenbaum et al., (Curr. Opin. Struct. Biol. 9:530-535 [1999]), the entire content of which is incorporated herein by cross reference. Approaches that can be taken include the following as examples:

1. Modification of the amino acid side chains to increase the hydrophobicity of defined regions of the peptide. For example, substitution of hydrogens with methyl groups on the phenylalanine at position 5 of the pentapeptide.

2. Substitution of the side chains with non-amino acids. For example, substitution of the phenylalanine at position 5 of the pentapeptide with other aryl groups.

3. Substitution of the amino- and/or carboxy-termini with novel substituents. For example, aliphatic groups to increase the hydrophobicity of the tripeptide DLF.

4. Modification of the backbone (amide bond surrogates), for example replacement of the nitrogens with carbon;

5. Modification of the backbone to introduce steric constraints, such as methyl groups.

6. Peptoids of N-substituted glycine residues.

7. Substitution of one or more L amino acids in the peptide sequences with D amino acids.

8. Substitution of one or more α-amino acids in the peptide sequences with β-amino acids or γ-amino acids.

9. Retro-inverso peptides with reversed peptide bonds and D-amino acids assembled in reverse order with respect to the original sequence.

10. The use of non-peptide frameworks, such as steroids, saccharides, benzazepine 1,3,4-trisubstituted pyrrolidinone, pyridones and pyridopyrazines and others known in the art.

11. The insertion of spacer amino acids. For example, to generate peptides of the form X¹X⁵X², QxX³X¹X⁵X² and QL X³X¹X⁵X² where X¹ is L, M, I or F, X² is L, I, V, C, F, W, P, D, A or G, X³ is D or S, and X⁵ is A, S, G, T, D or P. Particularly preferred hexapeptides containing this motif are shown in Table 13. A hexapeptide is in effect a “natural” mimetic of a pentapeptide with a single amino acid-residue spacer.

12. The use of approaches 1 to 10 with the peptides described at 11.

The interaction partner of the second embodiment includes the following compounds:

(i) a eubacterial β protein per se, or at least a portion of the domain thereof that includes at least a functional portion of the surface of the domain as defined in the first embodiment;

(ii) a mimetic of the interaction partner as defined in (i);

(iii) a peptide as defined above, or a polypeptide including at least one copy of the foregoing peptide; and

(iv) a compound that binds to the peptide of (iii).

With regard to a mimetic of item (ii) of the preceding paragraph, this can comprise a conformationally constrained linear or cyclic peptide that folds to mimic the disposition of the side chains of the amino acids in the native β protein or linked linear peptides representing in whole, or part, the discontinuous peptides comprising the surface. Conformational constrains may be obtained using disulphide bridges, amino acid derivatives with known structural constraints, non-amino acid frameworks and other approaches known to those skilled in the art, (Fairlie et al., Current Medicinal Chemistry (1998) 5:29-62, Stigers et al., Current Opinion in Chemical Biology (1999) 3:714-723). The mimetics can be antibodies, and related molecules, such as single chain antibodies, that bind in whole or in part to the peptides defined above, or mimetics of these peptides. The mimetics can comprise a protein engineered to express this site or region of β, or any chemical form that provides substituents in the appropriate positions to mimic side chains of the residues making up the peptides. These molecules can include modifications as described in 1-12 above.

In addition to the designed structural mimetics of the interacting peptides and the surface of β as described above, other mimetics can also be designed or selected. These include compounds that bind to the peptides defined above, including those designed/identified by structural modelling/determination of the peptides, the proteins in which they occur, or of eubacterial δ proteins. Also included are compounds that bind to β and occupy or occlude (in whole or in part) the structural space defined by the published co-ordinates in the 3D structure of E. coli β (Kong et al., Cell (1992) 69: 425-437) of the amino acid residues identified in the second embodiment or by modelling and/or structural determination of the equivalent positions in the orthologues of β from other species of eubacteria. Such mimetics may mimic the function, but not necessarily the structure of the peptides. Such mimetics could be identified by methods including screening of natural products, the production of phage display libraries (Sidhu et al., Methods in Enzymology (2000) 328:333-363), minimized proteins (Cunningham and Wells, Current Opinion in Structural Biology (1997) 7:457-462), SELEX (Aptamer) selection (Drolet et al., Comb. Chem. High Throughput Screen (1999) 2:271-278), combinatorial libraries and focussed combinatorial libraries, virtual screening/database searching (Bissantz et al., J. Med. Chem. (2000) 43:4759-4767) and rational drug design as known to those skilled in the art (Houghten et al., Drug Discovery Today (2000) 5:276-285). Such combinatorial libraries could be based on the peptide sequences—or their preferred forms as set out above—subjected to combinatorial variation as known to a medicinal chemist skilled in the art, or based upon the predictions of computer programs used for drug design (for example components of the InsightII and Cerius2 environments from MSI and the SYBYL Interface from Tripos). The libraries would be designed to include an adequate sampling of the range and nature of compounds likely to bind to β and occupy or occlude (in whole or in part) the structural space as defined above. For example the method of Erlanson et al., (Proc. Natl. Acad. Sci. (2000) 97:9367-9372) utilising the Ser345Cys mutant of E. coli β as described in example 9, or equivalent mutants of other eubacterial β proteins, to tether compounds adjacent to the binding site on β could be combined with the combinatorial target-guided ligand assembly of Maly et al., (Proc. Natl. Acad. Sci. (2000) 97:2419-2424) utilising, as an example, phenylalanine or the preferred dipeptides to efficiently nucleate the synthesis of mimetics of the peptides.

Compounds that can be utilised as test compounds in the method of the second embodiment include the following:

(i) a peptide as defined above, or a polypeptide that includes at least one copy of the peptide;

(ii) a mimetic of the peptide of (i);

(iii) a mimetic of at least part of the binding surface as defined in the second embodiment that retains at least part of the binding function of the whole surface;

(iv) a natural product or chemical compound that binds (i) or (ii);

(v) a natural product or chemical compound that binds in whole or in part to the binding surface of β protein as defined in the first embodiment; and

(vi) any compound that binds to either or both of the ligand and the interaction partner used in the assay.

It will of course be appreciated that when the ligand or interaction partner is a mimetic of β protein or the binding surface thereof and the test compound is also a mimetic of either entity, the second-mentioned mimetic will be a different molecule to the mimetic of β protein or the binding surface.

The method of the second embodiment can be carried out using any technique by which receptor-ligand interactions can be assayed. For example, surface plasmon resonance; assays in solution or using a solid phase, where binding is measured by immunometric, radiometric, chromogenic, fluorogenic, luminescent, or any other means of detection; any chromographic or electrophoretic methods; NMR, cryoelectron microscopy, X-ray crystallography and/or any combination of these methods.

Advantageously, in the method of the second embodiment, either component (i) or (ii) is immobilised on a solid support. The other component can be labelled so that binding of that component to the immobilised other component can be detected. Suitable labels will be known to one of skill in the art, as will suitable solid supports. Typically, the label is a radioactive label such as ³⁵ incorporated into the compound comprising either component (i) or (ii). Alternatively the component in solution may be detected by binding of antibodies specific for the component and suitable development known to one of skill in the art.

A typical procedure according to the second embodiment is carried out as follows. In this procedure, the ligand for β protein is α protein. The purified α subunit protein is adsorbed onto the wells of a microtitre plate. The β subunit protein, with or without test compound, is added to the α adsorbed wells and incubated. The plate is washed free of unbound protein, and incubated with antibody specific for the β subunit. The bound antibody is then detected with a species specific Ig-horseradish peroxidase conjugate and appropriate substrate. The chromogenic product is measured at the relevant wavelength using a plate reader.

Turning to the third embodiment of the invention, the ligand and interaction partner can be any of the ligands and interaction partners used in conjunction with the second embodiment that can be expressed, including transient expression, in a host cell. The cell does not necessarily have to be genetically modified to express the ligand or interaction partner, which entities can be introduced into the cell using liposomes or the like. Advantageously, the ligand is a peptide selected from those defined above, a polypeptide including at least one copy of such a peptide, or a mimetic of the foregoing compounds. Similarly, the interaction partner is a eubacterial β protein per se, or at least a portion of the domain thereof that includes at least a functional portion of the surface of the domain as defined in the first embodiment. The interaction partner is advantageously also a mimetic of the compounds specified in the previous sentence.

The modified host of the method of the third embodiment can be an animal, plant, fungal or bacterial cell, a bacteriophage or a virus. Methods for modifying such hosts are generally known in the art and are described, for example, in Molecular Cloning A Laboratory Manual (J. Sambrook et al., eds), Second Edition (1989), Cold Spring Harbor Laboratory Press, the entire content of which is incorporated herein by cross-reference.

So that the inhibition or potentiation of the interaction between the β protein and ligand can be easily assessed, the host is advantageously engineered to include an indicator system. Such indicator systems are well known in the art. A preferred indicator system is the β-galactosidase reporter system.

A preferred procedure for carrying out the method of the third embodiment is by the modification of the yeast two-hybrid assays described in Example 2 below. Compounds at appropriate concentrations are added to the growth medium prior to assay of β-galactosidase activity. Compounds that inhibit the interaction of the β-binding protein with β will reduce the amount of β-galactosidase activity observed.

With reference to the fourth embodiment of the invention, details of peptide sequences suitable for structure modelling are given herein. Those of skill in the art will be familiar with the modelling procedures by which structures can be provided.

In step (b)(i) of the method of the fourth embodiment, the portion of the consensus sequence can be a tripeptide. A particularly preferred tripeptide is DLF. In the step (b)(ii) method, the pentapeptide and hexapeptide sequences defined above are preferred. However, any of the peptides disclosed herein can be employed. The term “modelling” as used in the context of step (b)(ii) includes a determination of the structure of a peptide when bound to the surface of β-protein.

The assay procedures described above can advantageously be used in step (c) of the fourth embodiment method.

Regarding the fifth embodiment of the invention, the term “eubacterial infestation of a biological system” is used herein to denote: disease-causing infection of an animal, including humans; infection or infestation of plants and plant products such as seeds, fruit and flowers; infestation of foods and contamination of food production processes; infestation of fermentation processes; environmental contamination by a eubacterial species such as contamination of soil; and the like. The term should not be interpreted as limited to the foregoing situations, however, as the method is applicable to any situation where reduction or elimination of the number of a eubacterial species is desired.

Compounds used against a eubacterial infestation—that is, compounds that modulate the interaction between a eubacterial β protein and proteins that interact therewith—are preferably inhibitors of that interaction. However, modulator compounds that enhance the interaction between a eubacterial β protein and proteins that interact therewith can also be used against eubacterial infestations. In the latter circumstance, the efficacy of the compound lies in it inhibiting the release at the correct of a protein bound to β with disruption of cell replication. DNA replication requires the exchange of proteins on β, primarily the α and δ proteins of the replisome.

The term “infested” as used in the fifth embodiment and throughout the description embraces a systemic infection of eukaryotic organisms, such as animal, plants, fungi and sponges or surface infection thereof by a eubacterial species. The term also includes infections of parts of eukaryotic organisms such as infection of meat and plant products. The term further embraces an infection of a culture of microorganisms. The term further includes the presence of a eubacterial species in a process or on a surface in a physical environment.

The term “delivering” as used in the fifth embodiment and throughout the description embraces administering the inhibitor compound in such a manner that it is taken up by a subject animal, plant or microorganism infested with a eubacterial species. In this context the term includes applying the inhibitor compound to the infested surface or to an animal or plant although the inhibitor compound may not necessarily need to be taken up by the organism if the eubacterial infestation is limited to the surface thereof. The term also embraces genetically modifying an animal, plant or microorganism so that the inhibitor compound is expressed endogenously by the modified organism. The genetic modification can include a mechanism for the regulated expression of the inhibitor compound. For example, a gene or genes for expression of an inhibitor compound introduced into a plant can be under the control of a promoter that is responsive to eubacterial infestation of the plant. Methods for genetically modifying an animal, plant or microorganism to express the desired inhibitor compound will be known to those of skill in the art as will methods of controlling expression of the inhibitor compound. The term “delivering” further includes the physical delivery of a composition including the inhibitor compound onto a surface or into a physical environment such as by spraying, wiping or the like.

The amount of modulator compound administered will depend on the particular compound, the nature of the infested system, and the eubacterial species involved. Those of skill in the art of the application of antibacterials will be cognizant of the amount of a particular inhibitor compound to use.

Modulator compounds are typically administered as compositions comprising the compound and a suitable carrier substance. Compositions can also include excipients, adjuvants and bulking agents, or any other compound used in the preparation of pharmaceutical, veterinary and agricultural compositions, or compositions for environmental use. Compositions can also include additional active agents such as other antibacterials or therapeutic agents.

Compositions can be prepared as syrups, lotions, sprays, tablets, capsules, gels, creams, or mere solutions. The nature of the composition used, and the route of administration, will depend on the biological system subject to the infestation, and the nature of the infestation. For example, a eubacterial infection of a human would normally be treated by administration of tablets or capsules comprising a composition of the modulator compound, or in more extreme cases by injection of a solution containing a modulator compound.

Compositions can be prepared by any of the procedures known to those of skill in the art. The invention also includes within its scope use of a modulator of the interaction between eubacterial β protein and other proteins for the preparation of a medicament for reducing the effect of eubacterial infestation of a biological system.

As indicated above, the peptides of the invention can be used as templates for the design of modulators of the interaction of ligands with β protein. Such modulator compounds are advantageously mimetics of the peptide, as peptides or polypeptides may be prone to proteolytic degradation by the target eubacterium or an infected host. Nevertheless, polypeptides and peptides may have use in some circumstances.

With regard to mimetics of the peptides and the surface of the β protein, these can take any chemical form as described above.

It will be appreciated that efficacy of any designed modulator compound can be tested using the methods of the second or third embodiments. It will also be appreciated that the modulator compound utilised in the fifth embodiment can be a designed modulator compound, or any compound, or mixture of compounds, identified as an efficacious modulator through use of the methods of the second and third embodiments.

Non-limiting examples of the invention follow.

### EXAMPLE 1

In this example, we describe the identification of peptide motifs of replisomal proteins responsible for the interaction of the proteins with the processivity clamp, β.

### A. Methods

Analysis of Amino Acid Sequences

Alignments of amino acid sequences of the protein families were constructed by taking sequences from a number of sources. PSI-BLAST searches of the non-redundant database of proteins at the NCBI, BLAST searches of the unfinished and completed genomes at the following servers:

NCBI (http://www.ncbi.nlm.nih.gov/Microb_blast/unfinishedgenome.html),

TIGR (http://www.tigr.org/cgi-bin/BlastSearch/blast.cgi?),

Sanger Center (http://www.sanger.ac.uk/DataSearch/omniblast.shtml), and

DOE Joint Genome Institute (http://spider.jgi-psf.org/JGI_microbial/html).

Searches of non-redundant GenPept and B. subtilis open reading frames were undertaken using the Pattinprot server (http://pbil.ibcp.fr/cgi-bin/npsa_automat.p1?page=npsa_pattinprot.html). Predicted secondary structures were determined using the following servers:

PSIPRED at http://insulin.brunel.ac.uk/psipred), and

Jpred at http://jura.ebi.ac.uk:8888/submit.html.

Protein fold recognition was carried out using the 3D-PSSM server v2.5.1 at http://www.bmm.icnet.uk/˜3dpssm. Modelling was carried out using the SWISS-MODEL server at http://www.expasy.ch/swissmod/SM_FIRST.html. Models were manipulated using SWISS-MODEL and the Swiss-PdbViewer.

### B. Results

Eubacterial Polymerases DnaE, PolB and PolC Contain a Conserved Peptide Motif at the Carboxy-Terminus of their Polymerase Domains

The major eubacterial replicative polymerases, are the α subunits of DNA Polymerase III (DnaE and PolC). Whilst PolB is a repair polymerase, the carboxy-terminus of the eubacterial PolB proteins contains the short conserved peptide QLsLF. Inspection of the carboxy-termini of the members of the eubacterial PolC family of DNA Polymerases also identified a short peptide with the consensus sequence QLSLF (Seq. ID No. 622) at, or very close to, the carboxy-terminus of all members of the family so far identified. The results of this analysis are presented in Table 1 for the PolC1 family and in Table 2 for the PolB2 family. In these tables, and the following tables of sequence data, the residues comprising the motif are presented (second last column) as well as the ten residues on the N-terminal side of the motif, and up to the tenth residue on the C-terminal side of the motif where such residues occur. In both families the peptide is not predicted to be part of a helix or sheet and is predicted to be preceded by a helix. Thus, this motif is a good candidate for a β-binding site in the eubacterial enzymes.

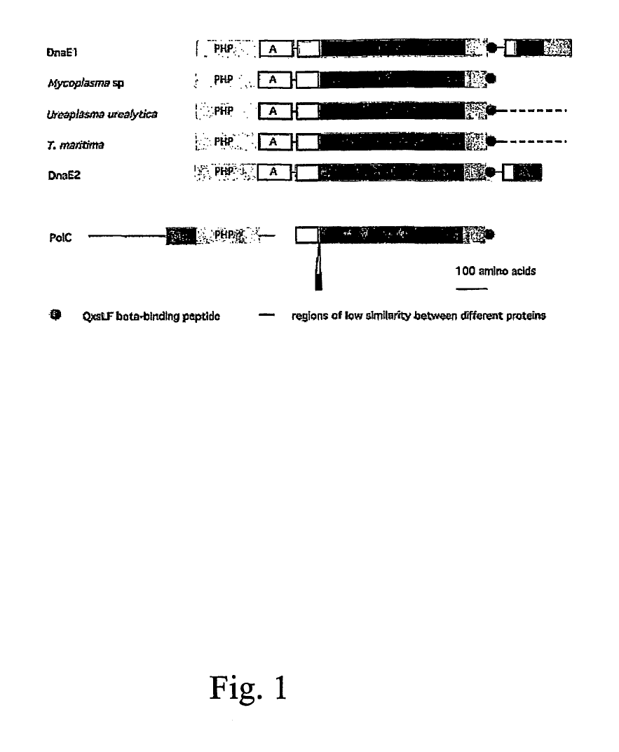

PolC is the α subunit of DNA Polymerase III in many gram-positive bacteria. However, in most bacteria DnaE is the α subunit. If the peptide QLsLF were indeed part of the β-binding site it should also be present in the DnaE subunit. The members of the DnaE and PolC families are related and contain similar domains, but are organised in slightly different ways (FIG. 1). The DnaE family can be further divided into the DnaE1 and DnaE2 subfamilies on the basis of their domain organisation (FIG. 1) and sequence similarities. Inspection of the carboxy-termini of the members of the DnaE1 and DnaE2 subfamilies did not identify any conserved peptide motif similar to QLsLF. Detailed analysis of the region immediately following the proposed helix-hairpin-helix domain (equivalent to the location of the QLsLF motif in the PolC enzymes) identified the short peptide with the consensus sequence QxsLF as equivalent to the motif identified in PolB and PolC. The data used for this analysis are presented in Tables 3 and 4. Structures shown were predicted using 3D-pssm with the E. coli DnaE1 sequenced used to initiate the alignment of sequences. Sequence data shown for the species Y. pestis, H. ducreyi, P. multocida, A. actinomycetemcomitans, S. putrefaciens, P. aeruginosa, P. putida L. pneumophila, T. ferroxidans, N. gonorrhoeae, B. brochiseptica, B. pertussis, R. sphaeroides, C. crescentus, D. vulgaris, G. sulfurreducens, M. leprae, M. avium, C. diptheriae, C. difficile, D. ethogenes, S. aureus, B. anthracis, E. faecalis, S. pneumoniae, S. pyogenes, C. acetobutylicum, T. denticola, C. tepidum and P. gingivalis, are preliminary data obtained from the unfinished genomes server at at the following NCBI site:

NCBI (http://www.ncbi.nlm.nih.gov/Microb_blast/unfinishedgenome.html).

Sequence data shown for the species N. europaea, E. faecium, R. palustris, P. marinus and N. punctiforme are preliminary data and were obtained from relevant unfinished genomes servers at the DOE Joint Genome Institute (http://spider.jgi-psf.org/JGI_microbial/html/).

In addition a small amino acid is favoured immediately preceding and following the central motif. The peptide is not predicted to be part of a helix or β-sheet and is predicted to be preceded by a helix.

Identification of a Peptide with the Consensus QLsLF in Members of the UmuC/DinB Family of Repair Polymerases.

E. coli DNA Polymerases IV and V have increased efficiency of DNA synthesis in the presence of β. The UmcC/DinB family can be further divided into four subfamilies on the basis of sequence similarities. The four subfamilies have been designated DinB1, DinB2, DinB3 and UmuC. Analysis of the sequences of members of the DinB1 subfamily (Polymerase IV) identified a somewhat conserved peptide motif (Table 5), with the very loose consensus QxsLF at, or close to, the carboxy-terminus of the proteins. Polymerase V is a multi-subunit enzyme containing two molecules of a cleaved version of UmuD, designated UmuD′ and UmuC, the polymerase subunit. The members of the UmuC subfamily contained the conserved peptide motif, QLNLF (Seq. ID No. 630), approximately sixty amino acids from the carboxy-terminus of the protein (Table 7). The UmuC subfamily includes the chromosomally encoded UmuC proteins and the plasmid encoded SamB, RulB, MucB, ImpB and RumB proteins. Members of a third subfamily, DinB2, present in plasmids and bacteriophages of gram positive bacteria also contained a conserved motif with the sequence QLSLF (Seq. ID No. 622) at the equivalent position to the motifs in the DinB and UmuC subfamilies (Table 6).

Identification of Putative β-Binding Sites in Proteins Involved in Mismatch Repair

The MutS superfamily is common to mismatch DNA repair systems across the evolutionary landscape. The MutS protein is involved in the initial recognition of mismatches. The MutS superfamily has been divided into two families, MutS1 and MutS2. In the eubacteria, single subfamilies of the MutS1 and MutS2 families have been identified. In the MutS1 family, a conserved peptide matching the β-binding motif was identified in most members of the family (Table 8). The motif lies in a region of amino acid sequence polymorphic in length and sequence lying between the conserved MutS domain and a short conserved domain specific to eubacteria at the carboxy-terminus of the proteins (Table 8). The peptide is not predicted to be part of a helix or sheet and is predicted to be preceded by a helix. Similar motifs were not identified in members of the MutS2 superfamily.

Determination of β-Binding Peptide Consensus Sequence

The frequency of each amino acid at each position of the aligned proposed β-binding peptides was plotted (FIG. 9). From this plot, the consensus sequence of the pentapeptide was determined to be QL[SD]LF where [SD] means either S or D (Seq. ID No's 582 and 584, respectively).

Other Eubacterial Proteins with Possible β-Binding Sites

The proposed β-binding sites have a number of common features; they are not in domains that are conserved across all members of a group of families of proteins, they are usually at the carboxy-terminus of the protein, they are in regions of variable amino acid sequence and length, they are in regions not predicted to be in helices or sheets, they are frequently preceded by a helix and although the tertiary structures of these proteins are not known the peptides are likely to be on the external surface of the proteins. The non-redundant GenPept protein sequence database was searched for proteins containing the sequence QLSLF (Seq. ID No. 622) and the B. subtilis protein sequence database was searched for the peptide sequences related to QLSLF. Hits in proteins known to be involved in DNA replication and repair were investigated in more detail.

The location and amino acid conservation of the peptide motif and of the flanking sequences and predicted secondary structure were evaluated against the features above. With one exception, no further families of proteins that met these criteria were identified. The one exception was a number of proteins in a family of RepA proteins encoded by plasmids E. coli RA1, Acidothiobacillus ferrooxidans pTF5 and Buchnera aphidicola pBPS2 (Table 9).

Members of the fourth subfamily of the UmuC/DinB superfamily, DinB3, exhibited a much lower level of conservation of the motif, but with a few exceptions the Q or LF parts of the motif were conserved (Table 10).

In addition, a probable β-binding site was identified at the carboxy-terminus in some, but not all, members of the Duf72 family of proteins of unknown function (Table 11). The Duf72 family (Pfam PF01904) is described at the following site:

Pfam (http://www.sanger.ac.uk/Software/Pfam/index.shtml)

and includes the E. coli YecE protein (NCBI gi:1788175) and the B. subtilis YunF protein (NCBI gi:2635736). Further members of the family were identified by BLAST searches of databases as described in the methods section.

Analysis of a family of proteins related to DnaA, here designated the DnaA2 family and exemplified by the E. coli YfgE protein (NCBI gi:1788842), identified a probable β binding site at the amino-terminus (Table 12). Again, further members of the family were identified by BLAST searches of databases as described in the methods section above.

Identification of a Second, Hexapeptide, Putative β-Binding Motif

Analysis of the sequences of the proposed DnaA2 β-binding motif suggested that a hexapeptide with the consensus sequence QLxLxh (where x is any amino acid and h is any hydrophobic amino acid) might constitute a second less common β-binding motif. Examples of a similar motif also occur at low frequency in some of the other families of proteins, as can be appreciated from the data of Table 13. Overall, the sequences appear to have the loose consensus sequence QxxLxh.

TABLE 1
PolC1 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 553 | 122 PolC1 Thermotoga maritima MSB8 | GVLGDLPETE | QFTLF | |
| 554 | 415 PolC1 Desulfitobacterium hafniense DCB-2 | DCLKGIPESD | QISFF | DLIS |
| 555 | 101 PolC1 Clostridium difficile 630 | GSLENMSERN | QLSLF | |
| 556 | 229 PolC1 Carboxydothermus hydrogenoformans | GCLKGLAPTS | QLVLF | A |
| | TIGR | | | |
| 557 | 227 PolC1 Bacillus halodurans C-125 | GCLEGLPESN | QLSLF | |
| 558 | 104 PolC1 Bacillus stearothermophilus 10 | GCLDSLPDHN | QLSLF | |
| 559 | 103 PolC1 Bacillus subtilis 168 | GCLESLPDQN | QLSLF | |
| 560 | 105 PolC1 Staphylococcus aureus | GSLPNLPDKA | QLSIF | DM |
| 561 | 228 PolC1 Staphylococcus epidermidis RP62A | GSLPDLPDKA | QLSIF | DM |
| 562 | 102 PolC1 Bacillus anthracis Ames | GCLGDLPDQN | QLSLF | |
| 563 | 946 PolC1 Listeria innocua Clip11262 | GCLEGLPDQN | QLSLF | |
| 564 | 947 PolC1 Listeria monocytogenes 4b | GCLEGLPDQN | QLSLF | |
| 565 | 948 PolC1 Listeria monocytogenes EGD-e | GCLEGLPDQN | QLSLF | |
| 566 | 106 PolC1 Enterococcus faecalis V583 | GVLKDLPDEN | QLSLF | DML |
| 567 | 632 PolC1 Enterococcus faecium DOE | GVLKDLPDEN | QLSLF | |
| 568 | 112 PolC1 Lactococcus lactis IL1403 | GVLEGMPDDN | QLSLF | DDFF |
| 569 | 108 PolC1 Streptococcus equi Sanger | GILGNMPDDN | QLSLF | DDFF |
| 570 | 107 PolC1 Streptococcus pyogenes M1_GAS | GILGNMPEDN | QLSLF | DDFF |
| 571 | 110 PolC1 Streptococcus mutans UA159 | GILGSMPEDN | QLSLF | DDFF |
| 572 | 111 PolC1 Streptococcus thermophilus | GILGNMPEDN | QLSLF | DDFF |
| 573 | 109 PolC1 Streptococcus pneumoniae type_4 | GILGNMPEDN | QLSLF | DELF |
| 574 | 113 PolC1 Ureaplasma urealyticum Serovar_3 | GVLDHLSETE | QLTLF | |
| 575 | 119 PolC1 Mycoplasma genitalium G-37 | QLFDEFEHQD | DHKLF N | |
| 576 | 120 PolC1 Mycoplasma pneumoniae M129 | LLDEFREQDN | QKKLF | |
| 577 | 114 PolC1 Mycoplasma pulmonis | GIFEQIPETN | QIFLI | |
| 578 | 121 PolC1 Clostridium acetobutylicum | GCLKGLPESD | QLSFF | DAI |
| | ATCC824D | | | |

TABLE 2
PolB2 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 405 | 125 PolB2 Chlorobium tepidum TLS | KPQDFSSIFS | ADTLF | AFSPEGIKVI |
| 406 | 414 PolB2 Anabaena sp. PCC7120 | APTTLESNKR | QLSLF | |
| 407 | 412 PolB2 Burkholderia cepacia LB400 | RDDFTALMSG | QKPLF | |
| 408 | 952 PolB2 Ralstonia metallidurans CH34 | DDDFETLLTG | QMTLF | PQ |
| 409 | 200 PolB2 Pseudomonas aeruginosa PAO1 | GDDFATLVDR | QMALF | |
| 410 | 201 PolB2 Pseudomonas putida KT2440 | GDDFARLTDH | QLLLF | |
| 411 | 226 PolB2 Pseudomonas syringae DC3000 | DDDFSTLIGG | QLGLF | |
| 412 | 411 PolB2 Pseudomonas fluorescens Pf0-1 | DDDFSTLIGG | QLGLF | |
| 413 | 202 PolB2 Shewanella putrefaciens MR-1 | KLNYTNIASK | QLSLI | |
| 414 | 199 PolB2 Vibrio cholerae N16961 | GKQFDELIAP | QLGLF | |
| 415 | 126 PolB2 Escherichia coli MG1655 | EDNFATLMTG | QLGLF | |
| 416 | 783 PolB2 Salmonella typhi CT18 | EDNFATLLTG | QLGLF | |
| 417 | 127 PolB2 Salmonella typhimurium LT2 | EDNFATVLTG | QLGLF | |
| 418 | 128 PolB2 Klebsiella pneumoniae MGH78578 | NDNFATIVTG | QLGLF | |
| 419 | 198 PolB2 Yersinia pestis CO-92 | QDDFTTLITG | QMGLF | |
| 420 | 124 PolB2 Geobacter sulfurreducens TIGR | MKKFAPFLPR | ERTLF | D |

TABLE 3
DnaE1 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 421 | 422 DnaE1 Magnetococcus sp. MC-1 | TQHQKDQKLG | FMNLF | GDEEAENSES |
| 422 | 197 DnaE1 Aquifex aeolicus VF5 | ANSEKALMAT | QNSLF | GAPKEEVEEL |
| 423 | 196 DnaE1 Thermotoga maritima MSB8 | NKRVEKDILE | IRSLF | GEKVEQESSN |
| 424 | 634 DnaE1 Chloroflexus aurantiacus J-10-fl | IEAQKAREIG | QSSLF | DIFGEATTAN |
| 425 | 195 DnaE1 Thermus aquaticus | AETRERGRSG | LVGLF | AEVEEPPLVE |
| 426 | 194 DnaE1 Deinococcus radiodurans R1 | AEINARAQSG | MSMMF | GMEEVKKERP |
| 427 | 193 DnaE1 Porphyromonas gingivalis W83 | SVVQEEKHSQ | SNSLF | GEEEDLMIPR |
| 428 | 674 DnaE1 Bacteroides fragilis NCTC9343 | NRYQADKAAA | VNSLF | GGDNVIDIAT |
| 429 | 421 DnaE1 Cytophaga hutchinsonii JGI | NAFQTEDDSN | QSSLF | GDSSSAKPAP |
| 430 | 192 DnaE1 Chlorobium tepidum TLS | QIQNKAVTLG | QGGFF | NDDFSDGQAG |
| 431 | 191 DnaE1 Chlamydia trachomatis | SREKKEAATG | VLTFF | SLDSMARDPV |
| 432 | 190 DnaE1 Chlamydophila pneumoniae | AKDKKEAASG | VMTFF | TLGAMDRKNE |
| 433 | 189 DnaE1 Nostoc punctiforme ATCC29133 | QSRAKDRASG | QGNLF | DLLGDGFSST |
| 434 | 1815 DnaE1 Anabaena sp. PCC7120 | QSRARDRASG | QGNLF | DLLGGYSSTN |
| 435 | 188 DnaE1 Synechocystia sp. PCC6803 | QKRAKEKETG | QLNIF | DSLTAGESIK |
| 436 | 187 DnaE1 Prochlorococcus marinus MED4 | SSRNRDRISG | QGNIJF | DSISKNDTKE |
| 437 | 972 DnaE1 Prochlorococcus marinus MIT9313 | ASRARDRLSG | QGNLF | DLVAGAADEQ |
| 438 | 934 DnaE1 Synechococcus sp. WH8102 | SSRAKDRDSG | QGNLF | DLMAAPNDED |
| 439 | 186 DnaE1 Treponema denticola TIGR | SQKKENESTG | QGSLF | EGSGIKEFSD |
| 440 | 185 DnaE1 Treponema pallidum Nichols | ARKKAVTSSR | QASLF | DETDLGECSE |
| 441 | 184 DnaE1 Borrelia burgdorferi B31 | SEDKNNKKLG | QNSLF | GALESQDPIQ |
| 442 | 423 DnaE1 Magnetospirillum magnetotacticum | AQAAEDRQSS | QMSLL | GGSNAPTLKL |
| | MS-1 | | | |
| 443 | 155 DnaE1 Rhodopseudomonas palustris CGA009 | QRNHEAATSG | QNDMF | GGLSDAPSII |
| 444 | 776 DnaE1 Mesorhizobium loti MAFF303099 | SLAQQNAVSG | QADIF | GASLGAQSQA |
| 445 | 639 DnaE1 Brucella suis 1330 | QRTQENAVSG | QSDIF | GLSGAPRETL |
| 446 | 971 DnaE1 Sinorhizobium meliloti 1021 | QRAQENKVSG | QSDMF | GAGAATGPEK |
| 447 | 933 DnaE1 Agrobacterium tumefaciens C58 | QMAQNNRTIG | QSDMF | GSGGGTGPEK |
| 448 | 157 DnaE1 Caulobacter crescentus TIGR | QSCHADRQGG | QGGLP | GSDPGAGRPR |
| 449 | 156 DnaE1 Rhodobacter sphaeroides 2.4.1 | AAIHEALNSS | QVSLF | GEAGADIPEP |
| 450 | 158 DnaE1 Rhodobacter capsulatus SB1003 | AAVAEAKSSA | QVSLF | GEAGDDLPPR |
| 451 | 935 DnaE1 Rickettsia conorii Malish_7 | TAYHEEQESN | QFSLI | KVSSLSPTIL |
| 452 | 161 DnaE1 Rickettsia helvetica | TSYHEEQESN | QLSLI | KVSSLSPTIL |
| 453 | 159 DnaE1 Rickettsia prowazekii Madrid_E | TSYHQEQESN | QFSLI | KVSSLSPTIL |
| 454 | 160 DnaE1 Rickettaia rickettsii | TAYHEEQESN | QFSLI | KVSSLSPTIL |
| 455 | 681 DnaE1 Cowdria ruminantium SANGER | EYNKYNSSFN | QISLF | NDKNHYKLVE |
| 456 | 970 DnaE1 Wolbachia sp. TIGR | NKNKQDKESS | QAALF | GSLDVLKPKL |
| 457 | 635 DnaE1 Sphingomonas aromaticivorans | EEASRSRTSG | QGGLF | GGDDHATPAT |
| | SMCC_F199 | | | |
| 458 | 151 DnaE1 Neisseria gonorrhoeae FA1090 | NADQKAANAN | QGGLF | DMMEDAIEPV |
| 459 | 150 DnaE1 Neisseria meningitidis Z2491 | NADQKAANAN | QGGLF | DMMEDAIEPV |
| 460 | 154 DnaE1 Nitrosomonas europaea | YAEQCSLAAS | QVSLF | DENTDLIQPP |
| | Schmidt_Stan_Watson | | | |
| 461 | 152 DnaE1 Bordetella bronchiseptica RB50 | AAEQAARSAN | QSSLF | GDDSGDVVAG |
| 462 | 153 DnaE1 Bordetella pertussis Tohama_I | AAEQAARSAN | QSSLF | GDDSGDVVAG |
| 463 | 677 DnaE1 Burkholderia pseudomallei K96243 | AAEQAAANAL | QAGLF | DIGGVPAHQH |
| 464 | 416 DnaE1 Burkholderia cepacia LB400 | AAEQASANAL | QAGLF | DMGDAPSQGH |
| 465 | 638 DnaE1 Burkholderia mallei ATCC23344 | AAEQAAANAL | QAGLF | DIGGVPAHQH |
| 466 | 424 DnaE1 Ralstonia metallidurans CH34 | LDRTEGESAN | QVSLF | DLMDDAGASH |
| 467 | 148 DnaE1 Acidothiobacillus ferrooxidans | AQFQSSQASL | QESLF | SGQEALRVAP |
| | ATCC23270 | | | |
| 468 | 149 DnaE1 Xylella fastidiosa | EQMSRERESG | QNPLF | GNADPSTPAI |
| | 8.1.b_clone_9.a.5.c | | | |
| 469 | 420 DnaE1 Xylella fastidiosa Ann-1 | EQMSRERESG | QNSLF | GNADPGTPAI |
| 470 | 419 DnaE1 Xylella fastidiosa Dixon | EQMSRERESG | QNSLF | GNADPGTPAI |
| 471 | 147 DnaE1 Legionella pneumophila | EKEHQNQSSG | QFDLF | SLLEDKADEQ |
| | Philadelphia-1 | | | |
| 472 | 641 DnaE1 Coxiella burnetii | EQRNRDMILG | QHDLF | GEEVKGIDED |
| | Nine_Mile_(RSA_493) | | | |
| 473 | 640 DnaE1 Methylococcus capsulatus TIGR | EQQGAMSAAG | QDDLF | GGFTAESPAA |
| 474 | 143 DnaE1 Pseudomonas aeruginosa PAO1 | EQTARSHDSG | HMDLF | GGVFAEPEAD |
| 475 | 145 DnaE1 Pseudomonas putida KT2440 | EQAAHTADSG | HVDLF | GSMFDAADVD |
| 476 | 231 DnaE1 Pseudomonas syringae DC3000 | EQTARSHDSG | HSDLF | GGLFVEADAD |
| 477 | 144 DnaE1 Pseudomonas fluorescens Pf0-1 | EQTARTRDSG | HADLF | GGLFVEEDAD |
| 478 | 142 DnaE1 Shewanella putrefaciens MR-1 | DQHAKAEAIG | QHDMF | GLLNSDPEDS |
| 479 | 141 DnaE1 Vibrio cholerae N16961 | SQHHQAEAFG | QADMF | GVLTDAPEEV |
| 480 | 139 DnaE1 Pasteurella multocida Pm70 | DQHAKDAAMG | QADMF | GVLTESHEDV |
| 481 | 137 DnaE1 Haemophilus influenzae KW20 | DQHAKDEAMG | QTDMF | GVLTETHEDV |
| 482 | 138 DnaE1 Haemophilus ducreyi 35000HP | DQHSKMEALG | QSDMF | GVLTETPEQV |
| 483 | 140 DnaE1 Actinobacillus | DQHAKDEALG | QVDMF | GVLTETNEEV |
| | actinomycetemcomitans HK1651 | | | |
| 484 | 230 DnaE1 Buchnera sp. APS | KESFRIKSFK | QDSLF | GIFQNELNQV |
| 485 | 134 DnaE1 Escherichia coli MG1655 | DQHAKAEAIG | QADMF | GVLAEEPEQI |
| 486 | 784 DnaE1 Salmonella typhi CT18 | DQHAKAEAIG | QTDMF | GVLAEEPEQI |
| 487 | 135 DnaE1 Salmonella typhimurium | DQHAKAEAIG | QTDMF | GVLAEEPEQI |
| 488 | 136 DnaE1 Yersinia pestis CO-92 | DQHAKAEAIG | QVDMF | GVLADAPEQV |
| 489 | 162 DnaE1 Desulfovibrio vulgaris | QKKLKERDSN | QVSLF | TMIKEEPKVC |
| | Hildenborough | | | |
| 490 | 164 DnaE1 Geobacter sulfurreducens TIGR | QKIQQEKESA | QVSLF | GAEEIVRTNG |
| 491 | 165 DnaE1 Helicobacter pylori | KDKANEMMQG | GNSLF | GAMEGGIKEQ |
| 492 | 163 DnaE1 Campylobacter jejuni NCTC11168 | RKMAEVRKNA | ASSLF | GEEELTSGVQ |
| 493 | 166 DnaE1 Streptomyces coelicolor A3 (2) | VAVKRKEAEG | QFDLF | GGMGDEQSDE |
| 494 | 167 DnaE1 Saccharopolyspora erythraea | IGLKRQQALG | QFDLF | GGGDDAGGEE |
| 495 | 425 DnaE1 Thermobifida fusca YX | LSSKKQEAHG | QFDLF | GGGDEEDGGE |
| 496 | 170 DnaE1 Mycobacterium avium 104 | LGTKKAEAMG | QFDLF | GGDGGCTESV |
| 497 | 169 DnaE1 Mycobacterium leprae TN | LGTKKAEAIG | QFDLF | GGTDGTDAVF |
| 498 | 973 DnaE1 Mycobactenium smegmatis MC2_155 | LGTKKAEAMG | QFDLF | GGGEDTGTDA |
| 499 | 168 DnaE1 Mycobacterium tuberculosis H37RV | LGTKKAEALG | QFDLF | GSNDDGTGTA |
| 500 | 682 DnaE1 Corynebacterium diptheriae | TSTKKAADKG | QFDLF | AGLGADAEEV |
| | NCTC13129 | | | |
| 501 | 172 DnaE1 Dehalococcoides ethenogenes TIGR | QREQKLKDSN | QTTMF | DLFGQQSPMP |
| 502 | 171 DnaE1 Clostridium difficile 630 | SMDRKKNVQG | QISLF | DAFGDSEEDS |
| 503 | 235 DnaE1 Carboxydothermus hydrogenoformans | EFYSKKSNGV | QLTLG | DFLPEADRYN |
| | TIGR | | | |
| 504 | 233 DnaE1 Bacillus halodurans C-125 | AEQVKEFQEN | TGGLF | QLSVEEPEYI |
| 505 | 785 DnaE1 Bacillus stearothermophilus 10 | IAIEHAQWVQ | ALEAG | GLSLKPKYAA |
| 506 | 173 DnaEl Bacillus subtilis 168 | HAELFAADDD | QMGLF | LDESFSIKPK |
| 507 | 174 DnaE1 Staphylococcus aureus COL | VLDGDLNIEQ | DGFLF | DILTPKQMYE |
| 508 | 234 DnaE1 Staphylococcus epidermidis RP62A | VLDLNSDVEQ | DEMLF | DLLTPKQSYE |
| 509 | 175 DnaE1 Bacillus anthracis Ames | LKGALEYANL | ARDLG | DAVPKSKYVQ |
| 510 | 937 DnaE1 Listeria innocua Clip11262 | YISLLGEDSK | GMNLF | AEDDDFLKKM |
| 511 | 936 DnaE1 Listeria monocytogenes 4b | YISLLGEDSK | GMNLF | AEDDDFLKKM |
| 512 | 939 DnaE1 Listeria monocytogenes EGD-e | YISLLGEDSK | GMNLF | AEDDEFLKKM |
| 513 | 176 DnaE1 Enterococcus faecalis V583 | NIQSILLSGG | SMDLL | ETLPKEEEIA |
| 514 | 177 DnaE1 Enterococcus faecium DOE | KIQNIVYSGG | SLDLL | GIMALKEEEV |
| 515 | 631 DnaE1 Lactococcus lactis IL1403 | ADHANLLNYY | SDDIF | MASSGGGFAY |
| 516 | 976 DnaE1 Streptococcus equi Sanger | LEGLLTFVNE | LGSLF | ADSSFSWVET |
| 517 | 179 DnaE1 Streptococcus pyogenes M1_GAS | LDGLLVFVNE | LGSLF | SDSSFSWVDT |
| 516 | 975 DnaE1 Streptococcus mutans UA159 | LEHLFTFVNE | LGSLF | ADSSYNWIEA |
| 519 | 178 DnaE1 Streptococcus pneumoniae type_4 | LANLFEFVKE | LGSLF | GDAIYSWQES |
| 520 | 180 DnaE1 Ureaplasma urealyticum Serovar_3 | EKTGLNGHFF | DLNLV | GLDYAKDMSV |
| 521 | 182 DnaE1 Mycoplasma genitalium G-37 | NDAKDFWIKS | DHLLF | TRMPLEKKDS |
| 522 | 181 DnaE1 Mycoplasma pneumoniae M129 | NLAKSFWVQS | NHELF | PKIPLDQPPV |
| 523 | 945 DnaE1 Mycoplasma pulmonis | LAKVQGDDID | ISNFF | QLEFSKNSSR |
| 524 | 183 DnaE1 Clostridium acetobutylicum | SGQRKKNLKG | QMNLF | TDFVQDDYEE |
| | ATCC824D | | | |

TABLE 4
DnaE2 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 525 | 664 DnaE2 Rhodopseudomonas palustris CGAOO9 | WAVRRLPDDV | PLPLF | EAASAREQED |
| 526 | 771 DnaE2 Mesorhizobium loti MAFF303099 | RALGAKSAAE | KLPLF | DQPALRLREL |
| 527 | 667 DnaE2 Brucella suis 1330 | WAVRRLPNDE | TLPLP | RAAAASELAQ |
| 528 | 944 DnaE2 Sinorhizobium meliloti 1021 | KALDEQSAVE | RLPLF | EGAGSDDLQI |
| 529 | 943 DnaE2 Sinorhizobium meliloti 1021 | LWAIKALRDE | PLPLF | TAAADREARA |
| 530 | 940 DnaE2 Agrobacterium tumefaciens C58 | LWAIKALRDE | PLPLF | AAAAIRENAV |
| 531 | 941 DnaE2 Agrobacterium tumefaciens C58 | LWAIKALRDE | PLPLF | AAAABREATA |
| 532 | 942 DnaE2 Agrobacterium tumefaciens C58 | LWAIKALRDE | PLPLF | AAAAEREMAA |
| 533 | 665 DnaE2 Caulobacter crescentus TIGR | GLKGEHKAPV | QAPLL | AGLPLFEERV |
| 534 | 668 DnaE2 Rhodobacter capsulatus SB1003 | WAVRAIRAPK | PLPLF | ANPLDGEGGI |
| 535 | 666 DnaE2 Sphingomonas aromaticivoraris | LWDVRRTPPT | QLPLF | AFANAPELGQ |
| | SMCC_F199 | | | |
| 536 | 684 DnaE2 Bordetella bronchiseptica RES0 | AWQAAASAQ | SRDLL | REAVIVETET |
| 537 | 683 DnaE2 Bordetella parapertussis 12822 | ASWQAAASAQ | SRDLL | REAVIVETET |
| 538 | 662 DnaE2 Bordetella pertussis Tohama_I | ASWQAAASAQ | SRDLL | REAVIVETET |
| 539 | 678 DnaE2 Burkholderia pseudomallei K96243 | ALWQAVAAAP | ERGLL | AAAPIDEAVR |
| 540 | 656 DnaE2 Burkholderia cepacia LB400 | RWWAVTAQHA | VPRLL | RDAPIAEAAL |
| 541 | 657 DnaE2 Ralstonia metallidurans CH34 | HARGAAVQTQ | HRDLL | HDAPPQEHAL |
| 542 | 661 DnaE2 Acidothiobacillus ferrooxidans | RHQALWAVQG | SLPLP | TALPMPVVPE |
| | ATCC23270 | | | |
| 543 | 663 DnaE2 Methylococcus capsulatus TIGR | AFWEAAGVEA | PTPLY | AEPQFAEAEP |
| 544 | 659 DnaE2 Pseudomonas aeruginosa PAO1 | ARWAVASVEP | QLPLF | AEGTAIEEST |
| 545 | 660 DnaE2 Pseudomonas putida KT2440 | ARWQVAAVQP | QLPLF | ADVQALPEEP |
| 546 | 787 DnaB2 Pseudomonas syringae DC3000 | ARWEVAGVEA | QRPLF | DDVTSEEVQV |
| 547 | 658 DnaE2 Pseudomonas fluorescens Pf0-1 | ARWEVAGVQK | QLGLF | AGLPSQEEPD |
| 548 | 671 DnaE2 Mycobacterium avium 104 | AGAAATQRPD | RLPGV | GSSSHIPALP |
| 549 | 672 DnaE2 Mycobacterium leprae TN | RAN | RLPGV | GGSSHIPVLP |
| 550 | 974 DnaE2 Mycobacterium smegmatis MC2_155 | AGAAATQRPD | RLPGV | GSSTHIPPLP |
| 551 | 670 DnaE2 Mycobacterium tuberculosis H37Rv | AGAAATGRPD | RLPGV | GSSSHIPALP |
| 552 | 673 DnaE2 Corynebacterium diptheriae | AGAAATEKAA | MLPGL | SMVSAPSLPG |
| | NCTC13129 | | | |

TABLE 5
DinB1 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 99 | 444 DinB1 Magnetococcus sp. MC-1 | SSQTATTQPQ | QLSLF | |
| 100 | 441 DinB1 Cytophaga hutchinsonii JGI | KLSNLVHGNY | QISLF | EDSEKNQNLY |
| 101 | 294 DinB1 Treponema denticola TIGR | MNIESDIPEA | QTELF | YSEKNVKKRK |
| 102 | 433 DinB1 Magnetospirillum magnetotacticum | TDLCPAEDAD | PPDLF | GPRPA |
| | MS-1 | | | |
| 103 | 434 DinB1 Magnetospirillum magnetotacticum | LGELSRTERR | QLDLL | TNDEPVRKRL |
| | MS-1 | | | |
| 104 | 266 DinB1 Methylobacterium extorquens AM1 | GDLCGAIHAD | RGDLA | DQGIERVARR |
| 105 | 432 DinB1 Rhodopseudomonas palustris CGA009 | SALTEQTGPA | EDDML | DRRSAHAERA |
| 106 | 775 DinB1 Mesorhizobium loti MAFF303099 | LGDVLPPDQR | QLRFEL | |
| 107 | 772 DinB1 Mesorhizobium loti MAFF303099 | SDLSDDDKAD | PPDLV | DVQSRKRAMA |
| 108 | 774 DinB1 Mesorhizobium loti MAFF303099 | VSHLEESAEL | QLDLPL | GLADEKRRPG |
| 109 | 650 DinB1 Brucella suis 1330 | SDLSPSDRAD | PPDLV | DIQATKRAVA |
| 110 | 930 DinB1 Sinorhizobium meliloti 1021 | SDLVDPDLAD | PPDLV | DPQASRRAAA |
| 111 | 242 DinB1 Sinorhizobium meliloti 1021 | LDTVDDRSEP | QLALAL | |
| 112 | 931 DinB1 Agrobacterium tumefaciens C58 | SDLRDAGLAD | PPDLV | DRQATRRAAA |
| 113 | 929 DinB1 Agrobacterium tumefaciens C58 | DQEAEDEEQP | QLDLAL | |
| 114 | 267 DinB1 Caulobacter crescentus TIGR | LTEFVDADTA | GADMF | ADEERRALKS |
| 115 | 435 DinB1 Rhodobacter sphaeroides 2.4.1 | AGAAEADLTG | TGDLL | DPNAGRRIAA |
| 116 | 265 DinB1 Rhodobacter capsulatus SB1003 | DLSPAGGRDP | IGDILL | DPQATARAAA |
| 117 | 643 DinB1 Sphingomonas aromaticivorans | AEDGPSGAAL | QAELPF | |
| | SMCC_F199 | | | |
| 118 | 263 DinB1 Neisseria gonorrhoeae FA1090 | GVGRLVPKNQ | QQDLW | A |
| 119 | 262 DinB1 Neisseria meningitidis Z2491 | GVGHLVPKNQ | QQDLW | A |
| 120 | 431 DinB1 Nitrosomonas europaea | SALLKENYYF | QEELF | |
| | Schmidt_Stan_Watson | | | |
| 121 | 264 DinB1 Bordetella pertussis Tohama_I | FPDAQAEAPR | QAELF | GDAF |
| 122 | 680 DinB1 Burkholderia pseudomallei K96243 | IDEDTAERHG | QIALF | |
| 123 | 430 DinB1 Burkholderia cepacia LB400 | ALTPPRRLPV | QADLP | FASDE |
| 124 | 644 DinB1 Burkholderia mallei ATCC23344 | IDEDTAERHG | QIALF | DDEDMSDEDA |
| 125 | 445 DinB1 Ralstonia metallidurans CH34 | ADQGDDPAPV | QEELRF | DAEPDSPVFR |
| 126 | 410 DinB1 Acidothiobacillus ferrooxidans | NVEAVPPEAL | QMNLL | EEPVDLR |
| | ATCC23270 | | | |
| 127 | 260 DinB1 Legionella pneumophila | LKQENTYQSV | QLPLL | DL |
| | Philadelphia-1 | | | |
| 128 | 645 DinB1 Coxiella burnetii | SFSEDPLLEL | QRTFEW | |
| | Nine_Mile_(RSA_493) | | | |
| 129 | 257 DinB1 Pseudomonas aeruginosa PAO1 | RLLDLQGAHE | QLRLF | |
| 130 | 258 DinB1 Pseudomonas putida KT2440 | RLRDLRGAHE | QLELF | PPK |
| 131 | 259 DinB1 Pseudomonas syringae DC3000 | RLHDLRDAHE | QLELF | ST |
| 132 | 428 DinB1 Pseudomonas fluorescens Pf0-1 | RLEDLRGGFE | QMELF | ER |
| 133 | 409 DinB1 Shewanella putrefaciens MR-1 | LISEVDPLQT | QLVLSI | |
| 134 | 256 DinB1 Vibrio cholerae N16961 | VMLKPELQMK | QLSMF | PSDGWQ |
| 135 | 248 DinB1 Pasteurella multocida Pm70 | PETTESKTQV | QMSLW | |
| 136 | 254 DinB1 Haemophilus influenzae KW20 | VNLPEENKQE | QMSLW | |
| 137 | 255 DinB1 Actinobacillus | VTLPEEKQSE | QMSLW | |
| | actinomycetemcomitans HK1651 | | | |
| 138 | 237 DinB1 Escherichia coli MG1655 | VTLLDPQMER | QLVLGL | |
| 139 | 238 DinB1 Salmonella typhi CT18 | VTLLDPQLER | QLVLGL | |
| 140 | 239 DinB1 Salmonella typhimurium LT2 | VTLLDPQLER | QLVLGL | |
| 141 | 240 DinB1 Klebsiella pneumoniae MGH78578 | VTLLDPQLER | QLLLGI | |
| 142 | 241 DinB1 Yersinia pestis CO-92 | VTLLDPQLER | QLLLDW | G |
| 143 | 270 DinB1 Desulfovibrio vulgaris | LGVSHFGGER | QMSLPI | GGMPRRDDTR |
| | Hildenborough | | | |
| 144 | 268 DinB1 Geobacter sulfurreducens TIGR | AISNLVHASE | QLPLF | PEERRLTTLS |
| 145 | 269 DinB1 Geobacter sulfurreducens TIGR | RITNLCYQRE | QLPLF | EKERRKALAT |
| 146 | 438 DinB1 Streptomyces coelicolor A3 (2) | SLTSAEHASH | QLTFDP | VDEKVRRIEE |
| 147 | 446 DinB1 Thermobifida fusca YX | GLVSADRVHH | QLALD | EEGPGWRAVE |
| 148 | 244 DinB1 Mycobacterium avium 104 | VSGIDRDGAQ | QLMLPF | EGRPPDAIDA |
| 149 | 272 DinB1 Mycobacterium avium 104 | VGFSGLSEVR | QESLF | PDLEMPAPQS |
| 150 | 245 DinB1 Mycobacterium smegmatis MC2_155 | VSNIDRGGTQ | QLELPF | AEQPDPVAID |
| 151 | 273 DinB1 Mycobacterium smegmatis MC2_155 | VGFSGLSDIR | QESLF | PDLEQPEEFP |
| 152 | 271 DinB1 Mycobacterium tuberculosis H37Rv | VGFSGLSDIR | QESLF | ADSDLTQETA |
| 153 | 274 DinB1 Corynebacterium diptheriae | VGLSGLEDAR | QDILF | PELDRVVPVK |
| | NCTC13129 | | | |
| 154 | 276 DinB1 Dehalococcoides ethenogenes TIGR | GISDFCGPEK | QLEIDP | ARARLEKLDA |
| 155 | 443 DinB1 Desulfitobacterium hafniense DCB-2 | TASRLQKGIE | QLSLF | QEESEEQTEL |
| 156 | 275 DinB1 Clostridium difficile 630 | NLSDKKETYK | DITLF | EYMDSIQM |
| 157 | 293 DinB1 Carboxydothermus hydrogenoformans | TPLVPVGGGR | QISLF | GEDLRRENLY |
| | TIGR | | | |
| 158 | 285 DinB1 Bacillus halodurans C-125 | DVIDKKYAYE | PLDLP | RYEEQIKQAT |
| 159 | 283 DinB1 Bacillus stearothermophilus 10 | HVFDEREEGK | QLDLF | RYEEEAKVEE |
| 160 | 282 DinB1 Bacillus subtilis 168 | DLVEKEQAYK | QLDLF | SFNEDAKDEP |
| 161 | 286 DinB1 Staphylococcus aureus COL | VGNLEQSTYK | NMTIY | DFI |
| 162 | 287 DinB1 Staphylococcus epidermidis RP62A | VGSLEQSDFK | NLTIY | DFI |
| 163 | 284 DinB1 Bacillus anthracis Ames | EIEWKTESVK | QLDLF | SFEEDAKEEP |
| 164 | 980 DinB1 Listeria innocua Clip11262 | VTNLKPVYFE | NLRLE | GL |
| 165 | 977 DinB1 Listeria monocytogenes 4b | VTNLKPVYFE | NLRLE | GL |
| 166 | 978 DinB1 Listeria monocytogenes EGD-e | VTNLKPVYFE | NLRLE | GL |
| 167 | 288 DinB1 Enterococcus faecalis V583 | NLDPLAYENI | VLPLW | EKS |
| 168 | 439 DinB1 Enterococcus faecium DOE | NLDPMTYENI | VLPLW | ENQEI |
| 169 | 779 DinB1 Lactococcus lactis IL1403 | GVTVTEFGAQ | KATLDM | Q |
| 170 | 932 DinB1 Streptococcus equi Sanger | TMTGLKDKVT | DILLD | LSFN |
| 171 | 247 DinB1 Streptococcus pyogenes M1_GAS | TMTMLEDKVA | DISLDL | |
| 172 | 440 DinB1 Streptococcus mutans UA159 | VTALEDSTRE | ELSLT | ADDFKT |
| 173 | 289 DinB1 Ureaplasma urealyticum Serovar_3 | KLVKKENVKK | QLFLF | D |
| 174 | 291 DinB1 Mycoplasma genitalium G-37 | LKKIDTDEGQ | KKSLF | YQFIPKSISK |
| 175 | 290 DinB1 Mycoplasma pneumoniae M129 | LKNNPSSSRP | EGLLF | YEYQQAKPKQ |
| 176 | 984 DinB1 Mycoplasma pulmonis | DFGDIYQSDL | SFDLF | DQKYDSKKEK |
| 177 | 292 DinB1 Clostridium acetobutylicum | LSGLCSGSSV | QISMF | DEKTDTRNEI |
| | ATCC824D | | | |

TABLE 6
DinB2 Protein Family Members

| | |
|---|---|
| Seq. ID | Sequence |

| | | | | |
|---|---|---|---|---|
| No. | Sequence name | N-term | Motif | C-term |

| | | | | |
|---|---|---|---|---|
| 178 | 987 DinB2 Fibrobacter succinogenes TIGR | ANNVLEATQE | SYDLF | TDVKKIEREK |
| 179 | 279 DinB2 Bacillus halodurans C-125 | LSNLTSDEAW | QLSFF | GNRDRAHQLG |
| 180 | 398 DinB2 Bacillus subtilis | LSNIEDDVNQ | QLSLF | EVDNEKRRKL |
| 181 | 277 DinB2 Bacillus subtilis 168 | LSQLSSDDIW | QLNLF | QDYAKKMSLG |
| 182 | 280 DinB2 Staphylococcus aureus COL | LSQFINEDER | QLSLF | EDEYQRKRDE |
| 183 | 281 DinB2 Staphylococcus epidermidis RP62A | LTQFIKESDR | QLNLF | IDEYERKKDV |
| 184 | 399 DinB2 Bacillus anthracis | LTNLLQEGEE | QISLF | DNVTQREQEV |
| 185 | 278 DinB2 Bacillus anthracis Ames | LTKLIGEGEE | QISLF | DNIIQREKEI |
| 186 | 981 DinB2 Listeria innocua Clip11262 | CGKLTLKTGL | QLNLF | EDATRTLNHE |
| 187 | 983 DinB2 Listeria innocua Clip11262 | CAGIKRKTSM | QLSVF | EDYTKTLQQE |
| 188 | 985 DinB2 Listeria monocytogenes 4b | CGKITLKTGL | QLNLF | EDATRTLNHE |
| 189 | 979 DinB2 Listeria monocytogenes EGD-e | CGKITLKTGL | QLNLF | EDFTQTLNHE |
| 190 | 401 DinB2 Enterococcus faecalis | YGRLVWNKNL | QLDLF | PVPEEQIHET |
| 191 | 998 DinB2 Enterococcus faecalis V583 | YGKLVWNESL | QLDLF | SEPEEQISEM |
| 192 | 997 DinB2 Enterococcus faecalis V583 | FGKLVWDTTL | QIDLF | SPPEEQIINN |
| 193 | 995 DinB2 Enterococcus faecium DOE | CSDLVYATGL | QLNLF | EDPEKQINEA |
| 194 | 996 DinB2 Enterococcus faecium DOE | CSKLVYSNAL | QLDLF | EDPNEQVKDL |
| 195 | 403 DinB2 Lactococcus lactis DCP3147 | GNQLSDSSVK | QLSLF | ESVQENQTNK |
| 196 | 402 DinB2 Lactococcus lactis DRC3 | ANNLIDEPYQ | LISLF | DSDEENEETI |
| 197 | 999 DinB2 Streptococcus gordonii | YSDFVDQEYG | LISLF | DDPLQVQKEE |
| 198 | 986 DinB2 Streptococcus gordonii | GNQLSDSSVK | QLSLF | ESVQENQTNK |
| 199 | 404 DinB2 Streptococcus pneumoniae SP1000 | YSGLVDESFG | LISLF | DDIEKIEKEE |

TABLE 7
UmuC Protein Family Members

| | |
|---|---|
| Seq. ID | Sequence |

| | | | | |
|---|---|---|---|---|
| No. | Sequence name | N-term | Motif | C-term |

| | | | | |
|---|---|---|---|---|
| 229 | 450 UmuC Magnetococcus sp. MC-1 | LLFLVSAQHF | QPSLF | APPPRLPNSR |
| 230 | 316 UmuC Porphyromonas gingivalis W83 | ILSDLVAEAY | QLNLF | DPIDRMRQER |
| 231 | 675 UmuC Bacteroides fragilis NCTC9343 | VIITEITDST | QLGLF | DSVDREKRKR |
| 232 | 451 UmuC Cytophaga hutchinsonii JGI | VSGIVPEDRV | QQNLF | DTVDRSKHNK |
| 233 | 452 UmuC Cytophaga hutchinsonii JGI | VIDIVPEEKI | QLNLF | EPQKNARLHA |
| 234 | 449 UmuC Prochlorococcus marinus MED4 | MQDLTNCKYL | QQSII | NYESQEESKK |
| 235 | 781 UmuC Prochlorococcus marinus MIT9313 | MQNLQSADHL | QQHLL | VAVHADEQHR |
| 236 | 448 UmuC Synechococcus sp. WH8102 | MQHLQGTELL | QSHLL | VPLSEAQQQR |
| 237 | 447 UmuC Methylobacterium extorquens AM1 | STDLVPLEAS | QRALI | GAFDRERGGA |
| 238 | 261 UmuC Acidothiobacillus ferrooxidans | LLEITSADAL | QADLF | LSAEEEARAH |
| | ATCC23270 | | | |
| 239 | 453 UmuC Legionella pneumophila | LEDLIPKKPR | QLDMF | HQPSDEHLKH |
| | Philadelphia-1 | | | |
| 240 | 454 UmuC Legionella pneumophila | LGDLIEKNCL | QLDLF | NQVSEKELNQ |
| | Philadelphia-1 | | | |
| 241 | 317 UmuC Pseudomonas syringae A2 | LMDICQPGEF | TDDLF | TIDQPASADR |
| 242 | 951 UmuC Shewanella putrefaciens 5/9/101 | LGDFYAPGVF | QLGLF | DEAKPQPKSK |
| 243 | 314 UmuC Shewanella putrefaciens MR-1 | LIELMPTKHI | QYDLF | HAPTENPALM |
| 244 | 307 UmuC Morganella morganii | MLSDLQGYET | QLDLF | SPAAVRPGSE |
| 245 | 309 UmuC Providencia rettgeri | LSDFYDPGMF | QPGLF | DDVSTRSNSQ |
| 246 | 305 UmuC Escherichia coli | MLADFSGKEA | QLDLF | DSATPSAGSE |
| 247 | 295 UmuC Escherichia coli MG1655 | LGDFFSQGVA | QLNLF | DDNAPRPGSE |
| 248 | 304 UmuC Shigella flexneri SA100 | LADFTPSGIA | QPGLF | DEIQPRKNSE |
| 249 | 310 UmuC Salmonella typhi CT18 | MLSSMTDGTE | QLSLF | DERPARRGSE |
| 250 | 301 UmuC Salmonella typhi CT18 | LNDFTPTGIS | QLNLF | DEVQPHERSE |
| 251 | 296 UmuC Salmonella typhi CT18 | LGGFFSQGVA | QLNLF | DDNAPRAGSA |
| 252 | 303 UmuC Salmonella typhimurium | LADFTPSGIA | QPGLF | DEIQPRKNSE |
| 253 | 306 UmuC Salmonella typhimurium | MLADFSGKEA | QLDLF | DSATPSAGSE |
| 254 | 302 UmuC Salmonella typhimurium | LNDFTPTGVS | QLNLF | DEVQPRERSE |
| 255 | 297 UmuC Salmonella typhimurium | LGDFFSQGVA | QLNLF | DDNAPRAGSA |
| 256 | 313 UmuC Klebsiella pneumoniae MGH78578 | LNDFTGSGVS | QLQLF | DERPPRPHSA |
| 257 | 298 UmuC Klebsiella pneumoniae MGH78578 | LGDFYSQGVA | QLNLF | DDNAPRKGSE |
| 258 | 299 UmuC Klebsiella pneumoniae MGH78578 | LGDFYSQGVA | QLNLF | DELAPRHNSA |
| 259 | 308 UmuC Serratia marcescens | MLSDLQGHET | QLDLF | APAAVRPGSE |
| 260 | 315 UmuC Desulfovibria vulgaris | LFGLEPAAGR | QGSLL | DLLDGSHEHK |
| | Hildenborough | | | |

TABLE 8
MutS1 Protein Family Sequences

| | |
|---|---|
| Seq. ID | Sequence |

| No. | Sequence name | N-term | Motif | C-term |
|---|---|---|---|---|
| 324 | 493 MutS1 Magnetococcus sp. MC-1 | QGHAPASQPY | QLTLF | EDAPPSPALL |
| 325 | 321 MutS1 Aquifex aeolicus VF5 | RELEEKENKK | EDIVP | IJIJEETFKKSE |
| 326 | 322 MutS1 Aquifex pyrophilus | LKELEGEKGK | QEVLP | FLEETYKKSV |
| 327 | 365 MutS1 Thermotoga maritima MSB8 | KNGKSNRFSQ | QIPLF | PV |
| 328 | 964 MutS1 Chloroflexus aurantiacus J-10-fl | VPAQETGQGM | QLSFF | DLAPHPVVEY |
| 329 | 364 MutS1 Porphyromonas gingivalis W83 | DEKGRSIDGY | QLSFF | QLDDPVLSQI |
| 330 | 676 MutS1 Bacteroides fragilis NCTC9343 | AEVSENRGGM | QLSFF | QLDDPILCQI |
| 331 | 473 MutS1 Cytophaga hutchinsonii JGI | KLKEVPKSTL | QMSLF | EAADPAWDSI |
| 332 | 363 MutS1 Chlorobium tepidum TLS | QALPLRVESR | QISLF | EEEESRLRKA |
| 333 | 361 MutS1 Chlamydia trachomatis D/UW-3/CX | DLRPEPEKAQ | QLVMF | |
| 334 | 362 MutS1 Chlamydophila pneumoniae | ITRPAQDKMQ | QLTLF | |
| 335 | 360 MutS1 Synechocystis sp. PCC6803 | AAEAAEDQAK | QLDIF | GF |
| 336 | 963 MutS1 Fibrobacter succinogenes TIGR | AQNKKIKAQP | QMDLF | APPDENTLLL |
| 337 | 359 MutS1 Treponema denticola TIGR | EKTPSSPAEK | GLSLF | PEEELILNEI |
| 338 | 358 MutS1 Treponema pallidum Nichols | AASKPCAQRV | SADLF | TQEELIGAEI |
| 339 | 357 MutS1 Borrelia burgdorferi B31 | VGREGNSCLE | FLPHV | SSDGNDKEIL |
| 340 | 474 MutS1 Magnetospirillum magnetotacticum | QASGMARLAD | DLPLF | AALAKPVAAS |
| | MS-1 | | | |
| 341 | 475 MutS1 Magnetospirillum magnetotacticum | RERPTRRRIE | DLPLF | ASLAAAPPPP |
| | MS-1 | | | |
| 342 | 476 MutS1 Rhodopseudomonas palustris CGA009 | DRGQPKTLID | DLPLF | AITARAPAEA |
| 343 | 777 MutS1 Mesorhizobium loti MAFF303099 | VSGKTNRLVD | DLPLF | SVAMKREAPK |
| 344 | 962 MutS1 Brucella suis 1330 | TSGKADRLID | DLPLF | SVMLQQEKPK |
| 345 | 343 MutS1 Sinorhizobium meliloti 1021 | RKNPASQLID | DLPLF | QVAVRREEAA |
| 346 | 953 MutS1 Agrobacterium tumefaciens C58 | RKNPASQLID | DLPLF | QIAVRREETR |
| 347 | 344 MutS1 Caulobacter crescentus TIGR | SKDQSPAKLD | DLPLF | AVSQAVAVTS |
| 348 | 477 MutS1 Rhodobacter sphaeroides 2.4.1 | SGGRRQTLID | DLPLF | RAAPPPPAPA |
| 349 | 955 MutS1 Rickettsia conorii Malish_7 | GKNILSTESN | NLSLF | YLEPNKTTIS |
| 350 | 342 MutS1 Rickettsia prowazekii Madrid_E | EKNILSNASN | NLSLF | NFEHEKPISN |
| 351 | 655 MutS1 Sphingomonas aromaticivorans | ATGGLAAGLD | DLPLF | AAAIEAAEEK |
| | SMCC_F199 | | | |
| 352 | 340 MutS1 Neisseria gonorrhoeae FA1090 | LENQAAANRP | QLDIF | STMPSEKGDE |
| 353 | 339 MutS1 Neisseria meningitidis Z2491 | LENQAAANRP | QLDIF | STMPSEKGDE |
| 354 | 478 MutS1 Nitrosomonas europaea | LEQETLSRSP | QQTLF | ETVEENAKAV |
| | Schmidt_Stan_Watson | | | |
| 355 | 341 MutS1 Bordetella bronchiseptica RB50 | RLEAQGAPTP | QLGLF | AAALDADVQS |
| 356 | 959 MutS1 Bordetella pertussis Tohama_I | RLEAQGAPTP | QLGLF | AAALDADVQS |
| 357 | 958 MutS1 Burkholderia pseudomallei K96243 | EQQSAAQATP | QLDLF | AAPPVVDEPE |
| 358 | 480 MutS1 Burkholderia cepacia LB400 | EQQSAAQPAP | QLDLF | AAPMPMLLED |
| 359 | 652 MutS1 Burkholderia mallei ATCC23344 | EQQSAAQATP | QLDLF | AAPPVVDEPE |
| 360 | 481 MutS1 Ralstonia metallidurans CH34 | EQSADATPTP | QMDLF | SAQSSPSADD |
| 361 | 337 MutS1 Acidothiobacillus ferrooxidans | RSSLSHTAPA | QLSLF | QAAPHPAVYR |
| | ATCC23270 | | | |
| 362 | 338 MutS1 Xylella fastidiosa | ITPLALDAPQ | QCSLF | ASAPSAAQEA |
| | 8.1.b_clone_9.a.5.c | | | |
| 363 | 483 MutS1 Xylella fastidiosa Ann-1 | ITPLALDAPQ | QCSLF | ASAPSAAQEA |
| 364 | 482 MutS1 Xylella fastidiosa Dixon | ITPLALDAPQ | QCSLF | ASAPSAAQEA |
| 365 | 336 MutS1 Legionella pneumophila | QIQDTQSILV | QTQII | KPPTSPVLTE |
| | Philadelphia-1 | | | |
| 366 | 654 MutS1 Coxiella burnetii | PVISETQQPQ | QNELF | LPIENPVLTQ |
| | Nine_Mile_(RSA_493) | | | |
| 367 | 651 MutS1 Methylococcus capsulatus TIGR | SAHQQAAPVA | QLDLF | LPPVVDEPEC |
| 368 | 331 MutS1 Pseudomonas aeruginosa PAO1 | QQSGKPASPM | QSDLF | ASLPHPVIDE |
| 369 | 332 MutS1 Azotobacter vinelandii OP | REAGKPQPPI | QSDLF | ASLPHPLMEE |
| 370 | 333 MutS1 Pseudomonas putida KT2440 | KAKDAPQVPH | QSDLF | ASLPHPAIEK |
| 371 | 957 MutS1 Pseudomonas syringae DC3000 | AKPGKPAIPQ | QSDMF | ASLPHPVLDE |
| 372 | 484 MutS1 Pseudomonas fluorescens Pf0-1 | AAKGKPAAPQ | QSDMF | ASLPHPVLDE |
| 373 | 319 MutS1 Shewanella putrefaciens MR-1 | HQVEGTKTPI | QTLLA | LPEPVENPAV |
| 374 | 485 MutS1 Vibrio parahaemolyticus | PRPSTVDVAN | QLSLI | PEPSEIEQAL |
| 375 | 326 MutS1 Vibrio cholerae N16961 | RKPSRVDIAN | QLSLI | PEPSAVEQAL |
| 376 | 327 MutS1 Pasteurella multocida Pm70 | DLRQLNQTQG | ELALM | EEDDSKTAVW |
| 377 | 328 MutS1 Haemophilus influenzae KW20 | IQDLRLLNQR | QGELF | FEQETDALRE |
| 378 | 329 MutS1 Haemophilus ducreyi 35000HP | QQTKMAQQHP | QADLL | FTVEMPEEEK |
| 379 | 330 MutS1 Actinobacillus | IQDLRLLNQR | QGELA | FESAEDENKD |
| | actinomycetemcomitans HK1651 | | | |
| 380 | 323 MutS1 Escherichia coli MG1655 | NAAATQVDGT | QMSLL | SVPEETSPAV |
| 381 | 487 MutS1 Salmonella enteritidis LK5 | NAAATQVDGT | QMSLL | AAPEETSPAV |
| 382 | 486 MutS1 Salmonella typhi CT18 | NAAATQVDGT | AMSLL | AAPEETSPAV |
| 383 | 324 MutS1 Salmonella typhimurium | NAAATQVDGT | QMSLL | AAPEETSPAV |
| 384 | 325 MutS1 Yersinia pestis CO-92 | NAAASTIDGS | QMTLL | NEEIPPAVEA |
| 385 | 488 MutS1 Yersinia pseudotuberculosis | NAAASTIDGS | QMTLL | NEEIPPAVEA |
| | IP32953 | | | |
| 386 | 966 MutS1 Geobacter sulfurreducens TIGR | KRAGAPKPSP | QLSLF | DQGDDLLRRR |
| 387 | 489 MutS1 Desulfitobacterium hafniense DCB-2 | EHLLNKEKAT | QLSLF | EVQPLDPLLQ |
| 388 | 490 MutS1 Clostridium difficile 630 | EDSVKEVALT | QISFD | SVNRDILSEE |
| 389 | 356 MutS1 Carboxydothermus hydrogenoformans | GLKVKDTVPV | QLSLF | EEKPEPSGVI |
| | TIGR | | | |
| 390 | 347 MutS1 Bacillus halodurans C-125 | KEVASTNEPT | QLSLF | EPEPLEAYKP |
| 392 | 491 MutS1 Bacillus stearothermophilus 10 | EGVLAEAAFE | QLSMF | PDLAPAPVEP |
| 392 | 345 MutS1 Bacillus subtilis 168 | QKPQVKEEPA | QLSFF | DEAEKPAETP |
| 393 | 348 MutS1 Staphylococcus aureus COL | TLSQKDFEQA | SFDLF | ENDQKSEIEL |
| 394 | 349 MutS1 Staphylococcus epidermidis RP62A | HTSNHNYEQA | TFDLF | DGYNQQSEVE |
| 395 | 346 MutS1 Bacillus anthracis Ames | ETKVDNEEES | QLSFF | GAEQSSKKQD |
| 396 | 960 MutS1 Listeria innocua Clip11262 | KQPEEIHEEV | QLSMF | PVEPEEKASS |
| 397 | 961 MutS1 Listeria monocytogenes EGD-e | KQPEEVHEEV | QLSMF | PLEPEKKASS |
| 398 | 350 MutS1 Enterococcus faecalis V583 | EVSEVHEETE | QLSLF | KEVSTEELSV |
| 399 | 492 MutS1 Enterococcus faecium DOE | IQDRVKEENQ | QLSLF | SELSENETEV |
| 400 | 351 MutS1 Streptococcus equi Sanger | VRETQQLANQ | QLSLF | TDDGSSSEII |
| 401 | 352 MutS1 Streptococcus pyogenes M1_GAS | VESSSAVRQG | QLSLF | GDEEKAHEIR |
| 402 | 353 MutS1 Streptococcus mutans UA159 | ETKESQPVEE | QLSLF | AIDNNYEELI |
| 403 | 354 MutS1 Streptococcus pneumoniae type_4 | PMRQTSAVTE | QISLF | DRAEEHPILA |
| 404 | 320 MutS1 Clostridium acetobutylicum | VKEEPKKDSY | QIDFN | YLERESILKE |
| | ATCC824D | | | |

TABLE 9
RepA Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 579 | 1002 RepA Acidothiobacillus ferrooxidans | PVSDTAFAGW | QLSLF | QGFLANTDDQ |
| 580 | 1001 RepA Buchnera aphidicola | | MLLF | KILQSKFKKD |
| 581 | 1000 RepA Escherichia coli | EKLDVIKDSP | QMSLF | EIIESPAKKD |

TABLE 10
DinB3 Protein Family Sequences

| | |
|---|---|
| Seq. ID | Sequence |

| | | | | |
|---|---|---|---|---|
| No. | Sequence name | N-term | Motif | C-term |

| | | | | |
|---|---|---|---|---|
| 200 | 993 DinB3 Magnetospirillum magnetotacticum | AEEVVPAGAE | QPRLW | GASSGEDARA |
| | MS-1 | | | |
| 201 | 467 DinB3 Methylobacterium extorquens AM1 | ASRVEPLAER | QNSHL | AAGQQAPDLA |
| 202 | 464 DinB3 Rhodopseudomonas palustris CGA009 | ASVSVAVTEA | QRGFD | TTAHQAEDVA |
| 203 | 773 DinB3 Mesorhizobium loti MAFF303099 | VLAAAAFDMA | QADLT | GEVTDDGADI |
| 204 | 648 DinB3 Brucella suis 1330 | ALRSSTVAQR | QTGLD | QHEEDEAGFS |
| 205 | 463 DinB3 Sinorhizobium meliloti 1021 | VLRSERLDPA | QQDFS | GAPDESQLLA |
| 206 | 990 DinB3 Agrobacterium tumefaciens C58 | AVMTEPLEEA | QKASA | LIGDDVTDVT |
| 207 | 988 DinB3 Agrobacterium tumefaciens C58 | ATHAEPLVAA | QARSS | LLDEGRAEIA |
| 208 | 989 DinB3 Agrobacterium tumefaciens C58 | AVMAEPLEER | QKSSS | LVEDEVTDVT |
| 209 | 468 DinB3 Caulobacter crescentus TIGR | AFAVEPMAAA | QARLD | ADAAASADET |
| 210 | 465 DinB3 Rhodobacter capsulatus SB1003 | ATRVEPLAPA | QLGTT | PAASPDRLAD |
| 211 | 649 DinB3 Sphingomonas aromaticivorans | LPVTEPLAAS | QPTLD | GSGQETTEVA |
| | SMCC_F199 | | | |
| 212 | 462 DinB3 Bordetella bronchiseptica RB50 | APDTVPQPAA | STCLF | PEPGGTPADH |
| 213 | 991 DinB3 Bordetella parapertussis 12822 | APDTVPQPAA | STCLF | PEPGGTPADH |
| 214 | 679 DinB3 Burkholderia pseudomallei K96243 | ATRVESVAPP | ADDLF | PEPGGTREAR |
| 215 | 459 DinB3 Burkholderia cepacia LB400 | ADQVGEYAGQ | SDTLF | PMPESDGDSI |
| 216 | 646 DinB3 Burkholderia mallei ATCC23344 | ATRIESVAPP | ADDLF | PEPGGTREAR |
| 217 | 460 DinB3 Ralstonia metallidurans CH34 | VEAMEICVPQ | SDSLF | PEPGAEPAEL |
| 218 | 461 DinB3 Acidothiobacillus ferrooxidans | ALAPQHWPGR | QATWW | QDGVEEARWQ |
| | ATCC23270 | | | |
| 219 | 647 DinB3 Methylococcus capsulatus TIGR | SADIQPFTLP | TADLF | TPGAAGGESW |
| 220 | 455 DinB3 Pseudomonas aeruginosa PAO1 | ARELPPFTPQ | HRELF | DERPQQYLGW |
| 221 | 456 DinB3 Pseudomonas putida KT2440 | AEDLPPFVPQ | HRELF | DERPQQYLGW |
| 222 | 457 DinB3 Pseudomonas syringae DC3000 | ARDLPDFVPA | HRELF | DERVQQTLPW |
| 223 | 458 DinB3 Pseudomonas fluorescens Pf0-1 | AEDLPSFVPQ | FQELF | DDRPQQTLPW |
| 224 | 992 DinB3 Mycobacterium avium 104 | AVEVVSAEAL | QLPLW | GGLG |
| 225 | 470 DinB3 Mycobacterium smegmatis MC2_155 | PVEVVSSAAL | QLPLW | GGIGEEDRLR |
| 226 | 469 DinB3 Mycobacterium tuberculosis H37Rv | VETVSASEGL | QLPLW | GGLGEQDRLR |
| 227 | 471 DinB3 Corynebacterium diptheriae | LRPYECMRPS | QPQLW | GTNKSDEESE |
| | NCTC13129 | | | |
| 228 | 994 DinB3 Corynebacterium glutamicum AHP-3 | PLECVPPDMA | SGGLW | DTGRSQQHVA |

TABLE 11

Duf72 Protein Family Sequences

| | |
|---|---|
| Seq. ID | Sequence |

| | | | | |
|---|---|---|---|---|
| No. | Sequence name | N-term | Motif | C-term |

| | | | | | |
|---|---|---|---|---|---|
| 300 | 850 Duf72 | Nostoc punctiforme ATCC29133 | PWNNLEHPPN | QLSLW | S |
| 301 | 851 Duf72 | Anabaena sp. PCC7120 | PWNHLDYPPH | QLNLW | |
| 302 | 843 Duf72 | Pseudomonas aeruginosa PAO1 | PEPIPAPEVE | QLGLL | |
| 303 | 927 Duf72 | Pseudomonas putida KT2440 | PELPRAPEVE | QLGLL | |
| 304 | 842 Duf72 | Pseudomonas syringae DC3000 | PELDRGPQVE | QLGLL | |
| 305 | 928 Duf72 | Pseudomonas fluorescens Pf0-1 | PELYREPAAE | QLGLL | |
| 306 | 845 Duf72 | Shewanella putrefaciens MR-1 | LDKKPEETST | QMGLSW | |
| 307 | 844 Duf72 | Vibrio cholerae N16961 | APFPVTPEQP | QLSMF | |
| 308 | 852 Duf72 | Pasteurella multocida Pm70 | VKPKPEFLTG | QQSLF | |
| 309 | 848 Duf72 | Escherichia coli MG1655 | EIGAVPAIPQ | QSSLF | |
| 310 | 847 Duf72 | Salmonella typhi CT18 | EIGTAPSIPQ | QSSLF | |
| 311 | 846 Duf72 | Salmonella typhimurium | EIGTAPSIPQ | QSSLF | |
| 312 | 849 Duf72 | Yersinia pestis CO-92 | TLPTAPDWPE | QETLF | |
| 313 | 835 Duf72 | Bacillus halodurans C-125 | EIEYRGLTPK | QLNLF | E |
| 314 | 836 Duf72 | Bacillus stearothermophilus 10 | GIEYTGLAPR | QLGLF | |
| 315 | 834 Duf72 | Bacillus subtilis 168 | DIEYSGLAPR | QLDLF | |
| 316 | 839 Duf72 | Staphylococcus aureus | NIEYEGLAPQ | QLKLF | |
| 317 | 838 Duf72 | Staphylococcus epidermidis RP62A | DIDYEGLAPQ | QLKLF | |
| 318 | 837 Duf72 | Bacillus anthracis Ames | NITYGEPKPE | QLNLF | E |
| 319 | 833 Duf72 | Listeria innocua Clip11262 | QVEFQGLAPM | QMDLF | SE |
| 320 | 832 Duf72 | Listeria monocytogenes | QVEFQGLAPM | QMDLF | SE |
| 321 | 853 Duf72 | Pediococcus acidilactici | GIHFTGLGPM | QLDLF | |
| 322 | 840 Duf72 | Enterococcus faecalis V583 | NLSYDDLNPK | QLDLF | |
| 323 | 841 Duf72 | Enterocoocus faecium DOE | NIKPDGLNPT | QMDLF | |

TABLE 12

DnaA2 Protein Family Sequences

| | |
|---|---|
| Seq. ID | Sequence |

| No. | Sequence name | N-term | Motif | C-term |
|---|---|---|---|---|
| 261 | 891 DnaA2 Magnetococcus ap. MC-1 | MHTGSA | QLLIAF | PLDPVLSWEN |
| 262 | 892 DnaA2 Magnetospirillum magnetotacticum | MSEA | QLPLAF | GHVPSLAAED |
| | MS-1 | | | |
| 263 | 894 DnaA2 Rhodopseudomonas palustris CGA009 | VEPR | QLALDL | PHAESLSRED |
| 264 | 895 DnaA2 Mesorhizobium loti MAFF303099 | MTAQRTDPPR | QLPLDL | GHGTGYSRDE |
| 265 | 896 DnaA2 Sinorhizobium meliloti 1021 | MKRHLSE | QLPLVF | GHAPATGRDD |
| 266 | 893 DnaA2 Agrobacterium tumefaciens C58 | KTDNARSKAE | QLPLAF | SHQSASGRED |
| 267 | 897 DnaA2 Caulobacter crescentus TIGR | MST | QFKLPL | ASPLTHGRED |
| 268 | 899 DnaA2 Rhodobacter sphaeroides 2.4.1 | VKG | QLAFDL | PIRPALSRED |
| 269 | 898 DnaA2 Rhodobacter capsulatus SB1003 | MTR | QLPLPL | PVRVAEGRED |
| 270 | 1812 DnaA2 Rickettsia conorii Malish_7 | VQ | QYIFRF | TTSSKYHPDE |
| 271 | 900 DnaA2 Rickettsia prowazekii Madrid_E | MQ | QYIFHF | TPSNKYHPDE |
| 272 | 1813 DnaA2 Wolbachia sp. TIGR | RKRLRKRFNV | QLNLF | NNNQADYSRQ |
| 273 | 902 DnaA2 Neisseria gonorrhoeae FA1090 | MN | QLIFDF | AAHDYPSFDK |
| 274 | 901 DnaA2 Neisseria meningitidis Z2491 | MN | QLIFDF | AAHDYPSFDK |
| 275 | 903 DnaA2 Nitrosomonas europaea | MR | QQLLDI | TEIGPPSLDN |
| | Schmidt_Stan_Watson | | | |
| 276 | 904 DnaA2 Bordetella parapertussis 12822 | MNR | QLLLDV | LPAPAPTLNN |
| 277 | 907 DnaA2 Burkholderia fungorum | VLR | QLTLDL | GTPPPSTFDN |
| 278 | 906 DnaA2 Burkholderia pseudomallei K96243 | VTR | QLTLDL | GTPPPSTFDN |
| 279 | 905 DnaA2 Burkholderia mallei ATCC23344 | VTR | QLTLDL | GTPPPSTFDN |
| 280 | 908 DnaA2 Ralstonia metallidurans CH34 | MSPRQK | QLSLEL | GSPPPSTFEN |
| 281 | 909 DnaA2 Acidothiobacillus ferrooxidans | MGNR | QRILPL | GVQAPATLEG |
| | ATCC23270 | | | |
| 282 | 910 DnaA2 Xylella fastidiosa | MSVS | QLPLAL | RYSSDQRFET |
| | 8.1.b_clone_9.a.5.c | | | |
| 283 | 911 DnaA2 Legionella pneumophila | MNK | QLALAI | KLNDEATLDD |
| | Philadelphia-1 | | | |
| 284 | 912 DnaA2 Coxiella burnetii | MID | QLPLRV | QLREETTFAN |
| | Nine_Mile_(RSA_493) | | | |
| 285 | 913 DnaA2 Methylococcus capsulatus TIGR | MAQ | QIPLHF | AVDPLQTFEA |
| 286 | 914 DnaA2 Pseudomonas aeruginosa PAO1 | MKPI | QLPLSV | RLRDDATFAN |
| 287 | 915 DnaA2 Pseudomonas putida KT2440 | MKPPI | QLPLGV | RLRDDATFIN |
| 288 | 916 DnaA2 Pseudomonas syringae DC3000 | MKPI | QLPLSV | RLRDDATFVN |
| 289 | 917 DnaA2 Pseudomonas fluorescens Pf0-1 | MKPI | QLPLGV | RLRDDATFIN |
| 290 | 919 DnaA2 Shewanella putrefaciens MR-1 | DVRVPLNSPL | QLSLPV | YLPDDETFNS |
| 291 | 918 DnaA2 Pasteurella multocida Pm70 | FVGCFLLENF | QLPLPI | HQLDDETLDN |
| 292 | 920 DnaA2 Haemophilus influenzae KW20 | MNK | QLPLPI | HQIDDATLEN |
| 293 | 921 DnaA2 Haemophilus ducreyi 35000HP | NWSIRFKNSL | QLLLPI | HQIDDETLDS |
| 294 | 922 DnaA2 Actinobacillus | MSEPHF | QLPLPI | HQLDDDTLEN |
| | actinomycetemcomitans HK1651 | | | |
| 295 | 923 DnaA2 Escherichia coli MG1655 | VEVSLNTPA | QLSLPL | YLPDDETFAS |
| 296 | 924 DnaA2 Salmonella typhi CT18 | VEVSLNTPA | QLSLPL | YLPDDETFAS |
| 297 | 925 DnaA2 Salmonella typhimurium | VEVSLNTPA | QLSLPL | YLPDDETFAS |
| 298 | 926 DnaA2 Yersinia pestis CO-92 | MVEVLLNTPA | QLSLPL | YLPDDETFAS |
| 299 | 1814 DnaA2 Geobacter sulfurreducens TIGR | ARSSRPFPAM | QLVFDF | PVTPKYSFDN |

TABLE 13

Hexapeptide Motif Sequences

| | |
|---|---|
| Seq. ID | Sequence |

| No. | Sequence name | N-term | Motif | C-term |
|---|---|---|---|---|
| 106 | 775 DinB1 Mesorhizobium loti MAFF303099 | LGDVLPPDQR | QLRFEL | |
| 108 | 774 DinB1 Mesorhizobium loti MAFF303099 | VSHLEESAEL | QLDLPL | GLADEKRRPG |
| 111 | 242 DinB1 Sinorhizobium meliloti 1021 | LDTVDDRSEP | QLALAL | |
| 113 | 929 DinB1 Agrobacterium tumefaciens C58 | DQEAEDEEQP | QLDLAL | |
| 117 | 643 DinB1 Sphingomonas aromaticivorans | AEDGPSGAAL | QAELPF | |
| | SMCC_F199 | | | |
| 125 | 445 DinB1 Ralstonia metallidurans CH34 | ADQGDDPAPV | QEELRF | DAEPDSPVFR |
| 128 | 645 DinB1 Coxiella burnetii | SFSEDPLLEL | QRTFEW | |
| | Nine_Mile_(RSA_493) | | | |
| 133 | 409 DinB1 Shewanella putrefaciens MR-1 | LISEVDPLQT | QLVLSI | |
| 138 | 237 DinB1 Escherichia coli MG1655 | VTLLDPQMER | QLVLGL | |
| 139 | 238 DinB1 Salmonella typhi CT18 | VTLLDPQLER | QLVLGL | |
| 140 | 239 DinB1 Salmonella typhimurium LT2 | VTLLDPQLER | QLVLGL | |
| 141 | 240 DinB1 Klebsiella pneumoniae MGH78578 | VTLLDPQLER | QLLLGI | |
| 142 | 241 DinB1 Yersinia pestis CO-92 | VTLLDPQLER | QLLLDW | G |
| 143 | 270 DinB1 Desulfovibrio vulgaris | LGVSHFGGER | QMSLPI | GGMPRRDDTR |
| | Hildenborough | | | |
| 146 | 438 DinB1 Streptomyces coelicolor A3 (2) | SLTSAEHASH | QLTFDP | VDEKVRRIEE |
| 148 | 244 DinB1 Mycobacterium avium 104 | VSGIDRDGAQ | QLMLPF | EGRPPDAIDA |
| 150 | 245 DinB1 Mycobacterium smegmatis MC2_155 | VSNIDRGGTQ | QLELPF | AEQPDPVAID |
| 154 | 276 DinB1 Dehalococcoides ethenogenes TIGR | GISDFCGPEK | QLEIDP | ARARLEKLDA |
| 169 | 779 DinB1 Lactococcus lactis IL1403 | GVTVTEFGAQ | KATLDM | Q |
| 171 | 247 DinB1 Streptococcus pyogenes M1_GAS | TMTMLEDKVA | DISLDL | |
| 261 | 891 DnaA2 Magnetococcus sp. MC-1 | MHTGSA | QLLIAF | PLDPVLSWEN |
| 262 | 892 DnaA2 Magnetospirillum magnetotacticum | MSEA | QLPLAF | GHVPSLAAED |
| | MS-1 | | | |
| 263 | 894 DnaA2 Rhodopseudomonas palustris CGA009 | VEPR | QLALDL | PHAESLSRED |
| 264 | 895 DnaA2 Mesorhizobium loti MAFF303099 | MTAQRTDPPR | QLPLDL | GHGTGYSRDE |
| 265 | 896 DnaA2 Sinorhizobium meliloti 1021 | MKRHLSE | QLPLVF | GHAPATGRDD |
| 266 | 893 DnaA2 Agrobacterium tumefaciens C58 | KTDNARSKAE | QLPLAF | SHQSASGRED |
| 267 | 897 DnaA2 Caulobacter crescentus TIGR | MST | QFKLPL | ASPLTHGRED |
| 268 | 899 DnaA2 Rhodobacter sphaeroides 2.4.1 | VKG | QLAFDL | PIRPALSRED |
| 269 | 898 DnaA2 Rhodobacter capsulatus SB1003 | MTR | QLPLPL | PVRVAEGRED |
| 270 | 1812 DnaA2 Rickettsia conorii Malish_7 | VQ | QYIFRF | TTSSKYHPDE |
| 271 | 900 DnaA2 Rickettsia prowazekii Madrid_E | MQ | QYIFHF | TPSNKYHPDE |
| 273 | 902 DnaA2 Neisseria gonorrhoeae FA1090 | MN | QLIFDF | AAHDYPSFDK |
| 274 | 901 DnaA2 Neisseria meningitidis Z2491 | MN | QLIFDF | AAHDYPSFDK |
| 275 | 903 DnaA2 Nitrosomonas europaea | MR | QQLLDI | TEIGPPSLDN |
| | Schmidt_Stan_Watson | | | |
| 276 | 904 DnaA2 Bordetella parapertussis 12822 | MNR | QLLLDV | LPAPAPTLNN |
| 277 | 907 DnaA2 Burkholderia fungorum | VLR | QLTLDL | GTPPPSTFDN |
| 278 | 906 DnaA2 Burkholderia pseudomallei K96243 | VTR | QLTLDL | GTPPPSTFDN |
| 279 | 905 DnaA2 Burkholderia mallei ATCC23344 | VTR | QLTLDL | GTPPPSTFDN |
| 280 | 908 DnaA2 Ralstonia metallidurans CH34 | MSPRQK | QLSLEL | GSPPPSTFEN |
| 281 | 909 DnaA2 Acidothiobacillus ferrooxidans | MGNR | QRILPL | GVQAPATLEG |
| | ATCC23270 | | | |
| 282 | 910 DnaA2 Xylella fastidiosa | MSVS | QLPLAL | RYSSDQRFET |
| | 8.1.b_clone_9.a.5.c | | | |
| 283 | 911 DnaA2 Legionella pneumophila | MNK | QLALAI | KLNDEATLDD |
| | Philadelphia-1 | | | |
| 284 | 912 DnaA2 Coxiella burnetii | MID | QLPLRV | QLREETTFAN |
| | Nine_Mile_(RSA_493) | | | |
| 285 | 913 DnaA2 Methylococcus capsulatus TIGR | MAQ | QIPLHF | AVDPLQTFEA |
| 286 | 914 DnaA2 Pseudomonas aeruginosa PAO1 | MKPI | QLPLSV | RLRDDATFAN |
| 287 | 915 DnaA2 Pseudomonas putida KT2440 | MKPPI | QLPLGV | RLRDDATFIN |
| 288 | 916 DnaA2 Pseudomonas syringae DC3000 | MKPI | QLPLSV | RLRDDATFVN |
| 289 | 917 DnaA2 Pseudomonas fluorescens Pf0-1 | MKPI | QLPLGV | RLRDDATFIN |
| 290 | 919 DnaA2 Shewanella putrefaciens MR-1 | DVRVPLNSPL | QLSLPV | YLPDDETFNS |
| 291 | 918 DnaA2 Pasteurella multocida Pm70 | FVGCFLLENF | QLPLPI | HQLDDETLDN |
| 292 | 920 DnaA2 Haemophilus influenzae KW20 | MNK | QLPLPI | HQIDDATLEN |
| 293 | 921 DnaA2 Haemophilus ducreyi 35000HP | NWSIRFKNSL | QLLLPI | HQIDDETLDS |
| 294 | 922 DnaA2 Actinobacillus | MSEPHF | QLPLPI | HQLDDDTLEN |
| | actinomycetemcomitans HK1651 | | | |
| 295 | 923 DnaA2 Escherichia coli MG1655 | VEVSLNTPA | QLSLPL | YLPDDETFAS |
| 296 | 924 DnaA2 Salmonella typhi CT18 | VEVSLNTPA | QLSLPL | YLPDDETFAS |
| 297 | 925 DnaA2 Salmonella typhimurium | VEVSLNTPA | QLSLPL | YLPDDETFAS |
| 298 | 926 DnaA2 Yersinia pestis CO-92 | MVEVLLNTPA | QLSLPL | YLPDDETFAS |
| 299 | 1814 DnaA2 Geobacter sulfurreducens TIGR | ARSSRPFPAM | QLVFDF | PVTPKYSFDN |
| 306 | 845 Duf72 Shewanella putrefaciens MR-1 | LDKKPEETST | QMGLSW | |

### EXAMPLE 2

In this example, we demonstrate that the peptide motifs identified in Example 1 are necessary and sufficient to enable the binding of proteins to β.

### A. Methods

Materials

E. coli XL-1Blue was used as host for all plasmid constructions. pLexA, pB42AD, p8op-lacZ vectors and yeast EGY48 cells were from the Matchmaker two-hybrid system (Clontech). Minimal synthetic dropout base media with 2% glucose (SD) or induction media containing 2% galactose and 1% raffinose (SG), and different drop out amino acid mixtures (CSM) were obtained from BIO 101. All enzymes used for cloning and PCR were from Promega.

Yeast Two-Hybrid Plasmid Construction

We used the yeast two-hybrid system based on the LexA DNA binding domain and the transactivation domain from the bacterial protein B42. The coding region of E. coli β was amplified by PCR from XL-1 Blue genomic DNA using Pfu DNA polymerase.

Oligonucleotide primers forward and reverse primers, respectively

| | |
|---|---|
| 5′-TGGCTGGAATTCAAATTTACCGTAGAACGT-3′ | (Seq. ID |
| | No. 582) |
| and | |
| 5′-AGTCCAGAATTCTTACAGTCTCATTGGCAT-3′ | (Seq. ID |
| | No. 583) |

for amplifying the β gene were flanked by EcoRI sites (underlined) that allowed cloning of the β gene in the EcoRI site of pB42AD creating a translational fusion with the B42 transcriptional activation domain. To construct various deletions of the DnaE gene in pLexA, the appropriate portion of the DnaE gene was amplified by PCR using Pfu DNA polymerase. The PCR primers used to generate DnaE (542-991) and DnaE (736-991) fragments were

5′-TTTGATGAATTCAAAAGCGACGTTGAATACGC-3′ (5′ primer starting at amino acid 542, Seq. ID No. 584),

5′-GCTTTGGAATTCGTGTCATATCAAACGTTATG-3′ (5′ primer starting at amino acid 736, Seq. ID No. 585), and

5′-GACTTTGAATTCTCGAGTTAACCACGTTCTGTCGGGTGCA-3′ (3′ primer, Seq. ID No. 586).

For construct DnaE (542-735), the primers

| | |
|---|---|
| 5′-TTTGATGAATTCAAAAGCGACGTTGAATACGC-3′ | (Seq. ID No. 587) |
| and | |
| 5′-GACTTTGAATTCTCGAGTTACATAACGTTTGATAAGTCAC-3′ | (Seq. ID No. 588) |

were used. All forward primers contained EcoRI sites (underlined) and reverse primers were flanked by XhoI sites (underlined) that allowed cloning of each DnaE PCR product into the EcoRI and XhoI sites of pLexA, creating an in frame fusion with the LexA DNA binding domain. For site directed mutagenesis, DnaE (736-991) fragment was cloned into pQE11 (Qiagen).

Mutations were introduced in this plasmid using the mutagenic primers 2HyKK1 with 2HyKK2 for the MF to KK mutation and 2HyPP1 with 2HyPP2 for the QF to PP mutation using QuikChange protocol (Stratagene). These primers had the following sequences:

| | | |
|---|---|---|
| 5′-GTCAGGCCGATAAAAAGGGCGTGCTGGCC-3′ | (2HyKK1,, | Seq. ID No. 589) |
| 5′-GCCAGCACGCCCTTTTTATCGGCCTGACC-3′ | (2HyKK2,, | Seq. ID No. 590) |
| 5′-GAAGCTATCGGTCCTGCCGATATGCCAGGCGTGCTGGCC-3′ | (2HyPP1,, | Seq. ID No. 591) |
| and | | |
| 5′-GGCCAGCACGCCTGGCATATCGGCACCACCGATAGCTTC-3′ | (2HyPP2,. | Seq. ID No. 592) |

PCR fragments containing the mutation were then subcloned into pLexA to generate pLexADnaE (736-991 KK) and pLexADnaE (736-991 PP) plasmids. To subclone peptides containing the β-binding regions, we amplified appropriate regions of DnaE, UmuC, DinB and MutS by PCR using Pfu DNA polymerase. The primers for these amplifications were as follows:

| | | |
|---|---|---|
| DnaE (908-931) | | |
| 5′-GGAAAGAATTCGGTCCGGCGGCAGATCAACACGCG-3′ | (forward,, | Seq. ID No. 593) |
| and | | |
| 5′-GATCAACTCGAGAGGACCTCCAGCTCCCGGCTCTTCGGCCAGCAC-3′ | (reverse,; | Seq. ID No. 594) |
| DnaE (896-919) | | |
| 5′-TCTCAAAGAATTCGCAGCGGGTGCGAGTCAGGGAGTCGCGCAG-3′ | (forward,, | Seq. ID No. 595) |
| and | | |
| 5′-AATCCACTCGAGGCCTCCACCGATAGCTTCCGCTTT-3′ | (reverse,; | Seq. ID No. 596) |
| UmuC | | |
| 5′-TCTCAAAGAATTCGCGGGTGCGAGTCAGGGAGTCGCGCAG-3′ | (forward,, | Seq. ID No. 597) |
| and | | |
| 5′-AATCCACTCGAGTCCCGGTGCGTTGTCATCGAA-3′ | (reverse,; | Seq. ID No. 598) |
| DinB | | |
| 5′-TCTCAAAGAATTCGCGGGTGCGCCGCAAATGGAAAGACAA-3′ | (forward,, | Seq. ID No. 599) |
| and | | |
| 5′-AATCCACTCGAGTCCAGCTCCTAATCCCAGCACCAGTTG-3′ | (reverse,; | Seq. ID No. 600) |
| MutS | | |
| 5′-TCTCAAAGCCGCCGCTACGCAAGTGG-3′ | (forward,, | Seq. ID No. 601) |
| and | | |
| 5′-AATCCACTCGAGTCCAGCTCCTGGTACTGACAGCAAAGAC-3′ | (reverse,. | Seq. ID No. 602) |

These PCR fragments were digested with EcoRI and XhoI (underlined) and were fused in frame to LexA binding domain through an GAG or AGA linker. For the construction of pLexAPolB, double stranded DNA encoding the linker GAG and the sequence QLGLF (Seq. ID No. 636) with flanking EcoRI and XhoI sites were subcloned into pLexA.

The DNA inserts and the cloning junctions in all plasmids were confirmed by sequencing.

Two-Hybrid Assay

Interaction between β and various LexA-fusion proteins were tested in yeast EGY48 containing a lacZ reporter gene (EGY48p80p-lacZ) by cotransformation of pLexA fusion plasmid and pB42ADβ plasmid using the Lithium acetate method. Cotransformants were plated in synthetic complete medium lacking appropriate supplements to maintain plasmid selection.

β-Galactosidase

Three to six transformants were patched onto indicator medium (SG/Gal/Raf/-His/-Leu/-Trp/-Ura with X-gal), grown at 30° C. and checked at 12 h intervals up to 96 h for development of blue colour. Results were compared with the positive (pLexA-53 with pB42AD-T) and negative controls (pLexA-Lam with pB42AD-T) performed in parallel. Cells were also inoculated and grown to mid-log phase in selective medium containing glucose or galactose. β-Galactosidase activity was estimated using Yeast β-Galactosidase kit (Pierce) and enzyme activity expressed in Miller units. All results were reproducible in at least two independent assays.

### B. Results

Analysis of the β-Binding Site in E. coli DnaE

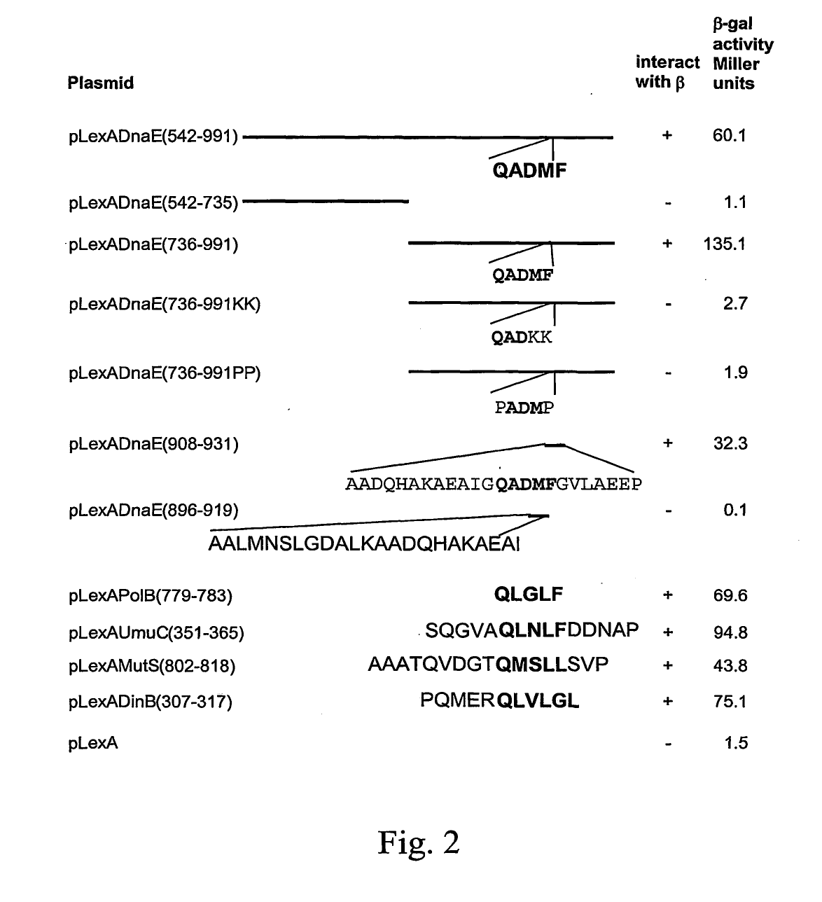

The foregoing bioinformatics analysis in Example 1 allowed identification of two short conserved peptide motifs in E. coli DnaE that fulfilled some of the criteria for being part of the β-binding site in eubacterial proteins. To obtain experimental verification of the role of the proposed peptide motifs a region of the gene encoding E. coli DnaE flanking the motif was cloned into the yeast two-hybrid vector pLexA to generate plasmid pLexADnaE (542-991) (FIG. 2). Significant expression of β-galactosidase was observed in Saccharomyces cerevisiae EGY48 transformed with plasmids pLexADnaE (542-991) and pB42ADβ expressing E. coli β fused to the transcription activator domain B42 (FIG. 2). Removal of the amino-terminal region that did not contain the proposed peptide increased the expression of β-galactosidase in the yeast two-hybrid system. No significant expression of β-galactosidase was observed from the fragment that did not contain the proposed binding peptide. To further characterise the proposed β-binding site, site-directed mutagenesis of the amino acids in the peptide motif was undertaken to convert the QADMF (Seq. ID No. 631) motif to QADKK (Seq. ID No. 632) (plasmid pLexADnaE (736-991 KK)) and PADMP (Seq. ID No. 633) (plasmid pLexADnaE (736-991 PP)), both predicted to be non-binding sequences. In S. cerevisiae transformed with plasmids pLexADnaE (736-991 KK) or pLexADnaE (736-99 PP1) and pB42ADβ, no significant expression of β-galactosidase was observed (FIG. 2). To further examine the role of the QADMF (Seq. ID No. 631) peptide a DNA fragment encoding a 24 amino acid peptide containing the sequence was inserted into the yeast two-hybrid vector pLexA to generate plasmid pLexADnaE (908-931), containing an in frame fusion of the peptide with LexA, again strong expression of β-galactosidase was observed from proteins containing the peptide and not from cells containing pLexADnaE (896-919) expressing LexA containing the adjacent peptide.

Analysis of the β-Binding Site in E. coli UmuC

The foregoing bioinformatics analysis in Example 1 allowed identification of a short conserved peptide motif in E. coli UmuC that appeared to fulfil all of the criteria for being part of the β-binding site in eubacterial proteins. To obtain experimental verification of the role of the proposed peptide motif a short peptide containing the motif (SQGVAQLNLFDDNAP, Seq. ID No. 637) was expressed as a LexA fusion in the plasmid pLexAUmuC(351-365). Significant expression of β-galactosidase was observed in S. cerevisiae EGY48 when pLexAUmuC (351-365) plasmid co-transformed with plasmid expressing B42-β fusion (FIG. 2).

Analysis of the β-Binding Site in E. coli DinB

The Example 1 analysis also allowed identification of a short conserved peptide motif in E. coli DinB that represents the hexapeptide β-binding peptide motif in eubacterial proteins. To obtain experimental verification of the role of the proposed variant peptide motif PQMERQLVLGL (Seq. ID No. 639), a short peptide containing the motif was expressed as a LexA fusion in the yeast two-hybrid vector pLexADinB (FIG. 2). Significant expression of β-galactosidase was observed in S. cerevisiae EGY48 when they were co-transformed with pLexADinB (307-317) plasmid and plasmid expressing B42-β fusion (FIG. 2).

Analysis of the β-Binding Site in E. coli MutS

The Example 1 analysis further allowed identification of a short conserved peptide motif in E. coli MutS that fulfilled all of the criteria for being part of the β-binding site in eubacterial proteins. To obtain experimental verification of the role of the proposed peptide motif, a short peptide encoding the motif “AAATQVDGTQMSLLSVP” (Seq. ID No. 638) was expressed as a LexA fusion in the yeast two-hybrid vector pLexAMutS(802-818) (FIG. 2). Significant expression of β-galactosidase was observed in S. cerevisiae EGY48 when they were co-transformed with pLexAMutS (802-818) plasmid and pB42ADβ plasmid FIG. 2). Consistent with the peptide results, the full-length E. coli MutS protein fused with LexA also interacted with E. coli β in the yeast two hybrid assay. Mutagenesis of LL (in the motif QMSLL: see Seq. ID No. 638) to AA in this peptide motif eliminated β binding by MutS.

Analysis of the β-Binding Site in E. coli PolB

From the Example 1 analysis, a short conserved peptide motif in E. coli PolB was identified that fulfilled all of the criteria for being part of the β-binding site in eubacterial proteins. To obtain experimental verification of the role of the proposed peptide motif a short peptide encoding the motif “QLGLF” (Seq. ID No. 636) was expressed as a LexA fusion in the yeast two-hybrid vector pLexAPolB(779-783) (FIG. 2). Significant expression of β-galactosidase was observed in S. cerevisiae when they were co-transformed with pLexAPolB (779-783) plasmid and pB42ADβ plasmid (FIG. 2).

### EXAMPLE 3

In this example, we describe the identification of a novel δ protein orthologue in Helicobacter pylori.

Search for Helicobacter pylori δ Orthologue

The complete amino acid sequence of the identified E. coli and Haemophilus influenzae δ orthologues was used to initiate the following searches: BLAST searches of the H. pylori complete genomes sequences, PSI-BLAST searches of the non-redundant database of proteins at the NCBI and BLAST searches of the unfinished and completed genomes at:

NCBI (http://www.ncbi.nlm.nih.gov/Microb_blast/unfinishedgenome.html),

TIGR (http://www.tigr.org/cgi-bin/BlastSearch/blast.cgi?),

Sanger Center (http://www.sanger.ac.uk/DataSearch/omniblast.shtml), and

DOE Joint Genome Institute (http://spider.jgi-psf.org/JGI_microbial/html/).

Searches were carried out on a reiterative basis using hits at the margins of significance to initiate new searches. For the δ protein the following criteria were used to determine whether or not to include a particular sequence in the next round of searching: product of similar length to known holA proteins, identities in similar relative positions in the proteins, proteins not currently assigned a function. This process was continued until a candidate putative orthologue of the δ protein had been identified in all bacteria for which a completed or substantially completed genome sequence was available. Additional searches were also undertaken using the SAM-T98 server at http://www.cse.ucsc.edu/research/compbio/HMM-apps/T98-query.html.

Bacterial and Yeast Strains

E. coli XL-1Blue was used as host for all plasmid constructions. BL21(DE3)pLysS (Novagen) was used for bacterial expression of the His₆ tagged proteins. S. cerevisiae strain EGY48 (MATa, his3, trp1, ura3, LexA _{op(X6)}-Leu) (Clontech) was used for the two hybrid analyses. Vector pET20b was from Novagen, pLexA and pBD42AD were from Clontech and pESC-LEU from Stratagene.

Cloning and Expression of Proteins

To generate various expression plasmids used in the in vitro protein interaction, the full length genes were amplified by PCR using a high fidelity polymerase Pfu DNA Polymerase (Promega). Human PCNA was amplified from Lambda ZAP colon cancer cDNA library (Stratagene) with the primers HuPCNA1 and HuPCNA2. The sequences of the foregoing primers and other primers are given in Table 14. In the table, restriction sites (NdeI, NotI, EcoRI and XhoI) are underlined and stop codons double underlined.

TABLE 14

Oligonucleotide primers

| | Seq. ID | |
|---|---|---|
| Primer | No. | Sequence |
| HuPCNA1 | 603 | 5′-GGGAATTCCATATGTTCGAGGCGCGCCTGG-3′ |
| HuPCNA2 | 604 | 5′-CGAAGCTTTGCGGCCGCCAGTCTCATTGGCATGAC-3′ |
| Hpδ1 | 605 | 5′-GGGAATTCCCATATGTATCGTAAAGATTTG-3′ |
| Hpδ2 | 606 | 5′-CCGCTCGAGTGCGGCCGCGGGGTTAATGATTTTTTGAAT-3′ |
| Hpδ′1 | 607 | 5′-GGGAATTCCATATGAAAAACTCCAACCGCCTT-3′ |
| Hpδ′2 | 608 | 5′-CCGCTCGAGTGCGGCCGCTGGCGTTTTCTTTTTGGATAA-3′ |
| Hpβ1 | 609 | 5′-GGGAATTCCATATGGAAATCAGTGTT-3′ |
| Hpβ2 | 610 | 5′-CGAAGCTTTGCGGCCGCTTATAGTGTGATTGGCAT-3′ |
| Ecβ1 | 611 | 5′-GGCATACATATGAAATTTACCGTAGAA-3′ |
| Ecβ2 | 612 | 5′-CTCGAGTGCGGCCGCTTACAGTCTTATTGGCATGA-3′ |
| Hphyδ1 | 613 | 5′-CTGGAATTCTATCGTAAAGATTTGGACCAT-3′ |
| Hphyδ2 | 614 | 5′-CCGCTCGAGTGCGGCCGCGGGGTTAATGATTTTTTGAAT-3′ |
| Hphyδ′1 | 615 | 5′-CTGGAATTCAAAAACTCCAACCGCCTTATT-3′ |
| Hphyδ′2 | 616 | 5′-CCGCTCGAGTGCGGCCGCTGGCGTTTTCTTTTTGGATAA-3′ |
| HylexA | 617 | 5′-CACTAAAGGGCGGCCGCATGAAAGCGTTAACGGCCAG-3′ |
| Hpτ1 | 618 | 5′-CGCCTCGAGATGCAAGTTTTAGCGTTAAAA-3′ |
| Hpτ2 | 619 | 5′-CGAGGAGCCTCGAGTCATAACAATTCCACGCTTTTG-3′ |

To construct pET-Hpδ, pET-Hpδ′, and pET-Hpβ, we carried out PCR reactions using H. pylori J99 genomic DNA as template with the pair of primers Hpδ1 and Hpδ2, Hpδ′1 and Hpδ′2; and Hpβ1 and Hpβ2 respectively (Table 14). E. coli β was amplified from genomic DNA of strain XL-1Blue with the primers Ecβ1 and Ecβ2 (Table 1). The resulting PCR fragments were digested with NdeI and NotI and cloned in the T7 promoter-based E. coli expression vector pET20b. The open reading frames (ORFs) of human PCNA, H. pylori δ and δ′ contained no stop codon and were inserted in front of the C-terminal His₆ tag in pET20b vector. In plasmids pET-Hpβ and pET-Ecβ, a stop codon was introduced before the NotI site and therefore expressed the native (non-tagged) proteins. All inserts and cloning junctions sequenced using an Applied Biosystems sequencer.

In Vitro Binding Assay

Radiolabelled (³⁵S-labeled) proteins were produced from various pET plasmids by in vitro transcription and translation using E. coli T7 S30 extract (Promega) and [³⁵S] methionine (Amersham Pharmacia Biotech) according to the manufacturer's recommendations. Radiolabelled His₆-tagged proteins (10-20 μl of the S30 extract reactions) were incubated for 1 h at 4° C. with 50 μl of 50% slurry of Ni-NTA resin in a total volume of 100 μl in binding buffer (50 mM NaH₂PO₄, 300 mM NaCl, 10 mM imidazole, pH8). The Ni-NTA beads were washed twice in the wash buffer (50 mM NaH₂PO₄, 300 mM NaCl, 20 mM imidazole pH8) and then resuspended in binding buffer BB14 (20 mM Tris pH 7.5, 0.1 mM EDTA, 25 mM NaCl, 10 mM MgCl₂) and then incubated with [³⁵S]methionine-labelled β. After 1 h incubation at RT, the beads were washed three times with the WB3 buffer (20 mM Tris pH 7.5, 0.1 mM EDTA, 0.05% Tween20) and proteins bound on the Ni-NTA beads were eluted by the addition of Laemmli sample buffer incubated for 5 min at 100° C. and were subjected to SDS-PAGE gel electrophoresis. Radiolabelled proteins were visualized by autoradiography with BioMaxTransScreen and BioMax MS film (Kodak).

Yeast Two-Hybrid System

Full-length ORFs of the H. pylori δ, τ and δ′ genes were obtained by PCR using gene-specific primers with flanking EcoRI and XhoI (Table 14). The PCR fragments were digested with EcoRI and XhoI and cloned into both pLexA and pB42AD vectors. Cloning into pLexA placed the H. pylori δ and δ′ ORFs in frame with the DNA-binding domain of LexA, downstream of the ADH promoter. Cloning into pB42AD placed the H. pylori δ and δ′ ORFs in frame with the B42 transcription activator domain and the C-terminal hem agglutinin (HA) epitope tag. For simultaneous expression of the LexA-δ and unfused τ proteins, a modified two-hybrid vector pESCLexHpδ/τ was constructed as follows. The DNA fragment containing the LexA DNA binding domain fused to the H. pylori δ ORF was PCR amplified from plasmid pLexAHpδ using the primers HyLexA and Hyδ 2 containing the NotI site, digested with Not I and inserted into the yeast dual expression vector pESC-LEU (Stratagene) to obtain pESCLexAδ. Finally, the H. pylori τ ORF was amplified by PCR using the primers Hyτ1 and Hyτ2 (Table 14), digested with XhoI and cloned into pESCLexAδ digested with XhoI. The resulting plasmid, pESCLexAδ/τ, coexpressed the LexAδ fusion protein from the yeast GAL10 promoter and the c-myc epitope tagged τ from the GAL1 promoter.

β-Galactosidase

Three to six transformants were patched onto selective medium and grown for 1 day at 30° C. when they were inoculated and grown to mid-log phase in selective medium containing glucose or galactose as indicated. β-galactosidase activity was assayed using Yeast β-Galactosidase kit (Pierce) and expressed in Miller units.

Co-Immunoprecipitation and Western Blotting

Yeast cells were allowed to grow in 50 ml of minimal medium containing 2% D(+) raffinose to an OD₆₀₀ up to 0.7 when shifted to a medium containing 2% D(+) galactose in order to induce Gal1/10 promoter. For protein extraction, yeast cells were harvested at OD₆₀₀ of 1.0 (approximately 1×10⁷ cells/ml) and collected by centrifugation and resuspended in ice-cold lysis buffer (50 mM Hepes, pH 7.5, 150 mM NaCl, 1.5 mM MgCl₂, 0.2 mM EDTA, 25% glycerol, 1 mM DTT) containing 2 mM phenylmethysulonyl fluoride and complete protease inhibitor cocktail (Boehinger Mannheim). Approximately ⅓ volume of ice-cold glass beads were added, and the cells were broken by vortexing several times at 4° C. The lysed cells were centrifuged and the lysate transferred to a new tube. For co-immunoprecipitations, the lysates were incubated with specific antibodies (anti-HA, 12A5 from Boehringer Mannheim) at 4° C. After 2 h, protein A-Sepharose (Amersham Pharmacia Biotech) was added, and the mixture was incubated for a further 2 h at 4° C. The immunoprecipitates were washed in ice-cold washing solution containing 10 mM Tris-HCl, pH 7.0, 50 mM NaCl, 30 mM NaPP, 50 mM NaF, 2 mM EDTA and 1% Triton X-100. Proteins were separated on 10% SDS-PAGE gels and transferred to nitrocellulose membranes (Bio-Rad). The membranes were blocked with 3% blotto in PBST (phosphate-buffered saline plus 0.1% Tween 20) for 1 h and subsequently incubated with either a anti-LexA polyclonal antibody or a anti-myc monoclonal antibody (Invitrogen) for 1 h, washed in PBST, and incubated for 1 h with peroxidase-conjugated secondary antibody. The membranes were washed in PBST and developed with enhanced chemiluminescence (Pierce), followed by exposure to Hyperfilm ECL (Amersham Pharmacia Biotech).

### B. Results

Identification of a Gene Encoding a Putative Orthologue of δ from H. pylori

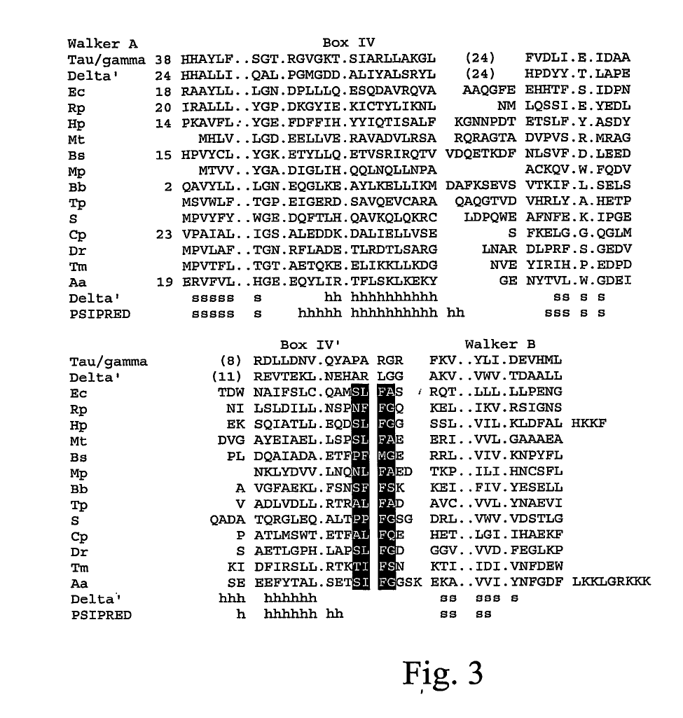

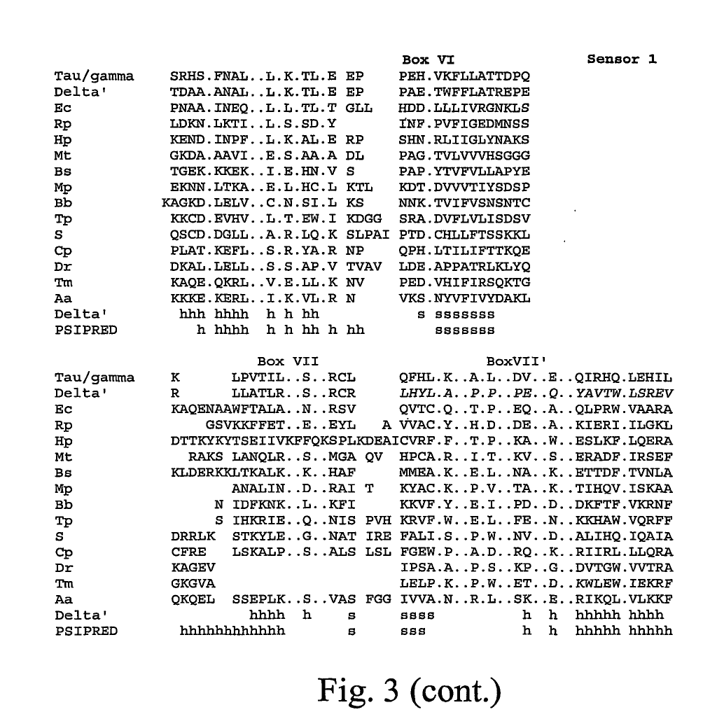

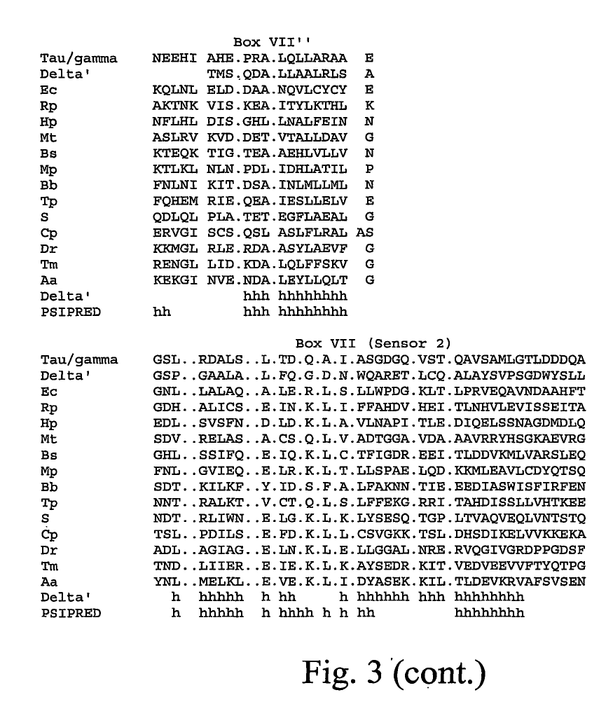

Initial BLAST searches of the translated complete genome sequence of H. pylori J99 with the E. coli and H. influenzae δ amino acid sequences failed to identify any significant matches. However, after a more extensive reiterative series of searches a family of proteins encoding putative orthologues of δ was identified. All bacteria with completed or substantially completed genome sequences contained a single gene encoding a member of the family, but most of the members of this family are currently not recognised as such. The alignment of the proposed orthologues of δ present in a range of bacteria with fully sequenced genomes is shown in FIG. 3. In FIG. 3, the amino acid sequences of the proposed degenerate AAA+ domain of the δ orthologues from E. coli (Ec), Rickettsia prowazeki (Rp), H. pylori J99 (Hp), Mycobacterium tuberculosis (Mt), Bacillus subtilis (Bs), Mycoplasma pneumoniae (Mp), Borrelia burgdorferi (Bb), Treponema pallidum (Tp), Synechocysitis sp. (S), Chlaymdia pneumoniae (Cp), Deinococcus radiodurans (Dr), Thermotoga maritima (Tm) and Aquifex aeolicus (Aa), are shown. The bracketed number is the number of amino acids missing from the alignment. The experimentally determined secondary structure of E. coli δ′ (Guenther et al., Cell (1997) 91:335-345) is shown, along with predicted secondary structure of E. coli δ determined using PSIPRED, s—sheet and h—helix. The members of the family are quite poorly conserved in amino acid sequence, with no amino acids being 100% conserved. The highly conserved positions are a glycine and a phenylalanine located close to the amino-terminus and an aspartic or glutamic acid and a lysine located close to the carboxy-terminus of the protein (FIG. 3). Unlike the δ′ and γ/τ families the sites with conservative substitutions are fairly well distributed across the whole length of the protein. The overall low level of conservation in such an important component of the clamp loader is probably due the apparent absence of enzymatic activities, with the δ subunit being primarily involved in protein-protein interactions.

The proposed H. pylori δ orthologue is encoded by gene jhp1168. The predicted protein exhibited low amino acid identity to the E. coli δ.

His₆ Tagged Helicobacter pylori δ can Bind δ

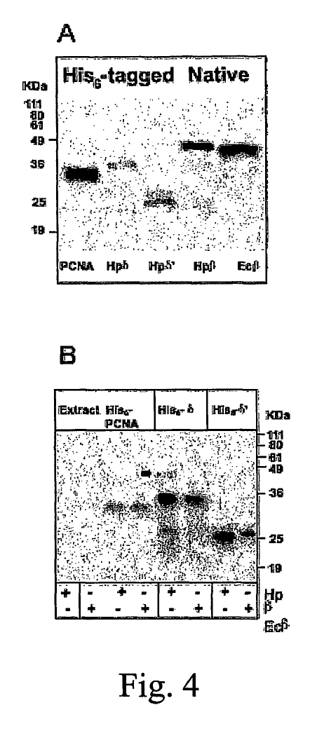

In order to confirm the identification of the putative δ orthologue in H. pylori, we first examined the interaction between H. pylori δ and the proposed β using an in vitro biochemical assay. Various H. pylori proteins δ, δ′, β and human PCNA (the eukaryote equivalent of the β subunit of DNA Polymerase III), and β from E. coli were expressed in E. coli using pET plasmids. To verify the δ-β interaction we used a protein interaction assays with one of the proteins immobilised on Ni-NTA beads. Proteins were synthesised in vitro from pET plasmids using E. coli T7 S30 extract and labelled with ³⁵S-methionine (FIG. 4). In FIG. 4A, proteins were synthesized by in vitro transcription-translation using E. coli T7 S30 extract from various pET plasmids. Translation efficiency was estimated by parallel reactions in the presence of [³⁵S]Met. Aliquots (5 μl) of the reaction mixtures were size-fractionated on 10% SDS/PAGE. The amount of proteins synthesized was quantitated by using a PhosphorImager and equal amounts were used in the binding experiments. In FIG. 4B, ³⁵S-labeled His₆-tagged human PCNA (lanes 3 and 4), H. pylori δ (lanes 5 and 6), and δ′ (lanes 7 and 8) (5-15 μl of reaction mixtures) were immobilised on Ni-NTA agarose beads. The beads were washed and incubated with 10 μl of the S30 extract reaction mixture containing the ³⁵S-labeled H. pylori β or E. coli β protein. Proteins associated with the resin were detected by SDS/PAGE on 10% gels followed by autoradiography. Lanes 1 and 2 are controls where reaction mixtures lacking plasmid template were used to bind Ni-NTA resin. The position of H. pylori β is indicated by an arrow. Each of the ³⁵S-labeled and His₆-tagged proteins were separately immobilised to Ni-NTA agarose beads via their His₆ tag. The Ni-NTA beads that carried immobilised S30 extract or each His₆-fusion proteins were washed and incubated with ³⁵S-labeled β protein. After washing, the ³⁵S-labeled proteins bound to the beads were eluted and analysed using SDS-PAGE followed by autoradiography. Typical results are shown in FIG. 4 and demonstrate that H. pylori β only bound to His₆δ. The binding is specific: H. pylori β did not bind to δ′ or to human PCNA. Moreover the interaction is species specific since E. coli β did not bind to H. pylori His₆-δ.

δ and δ′ Interact in the Presence of τ

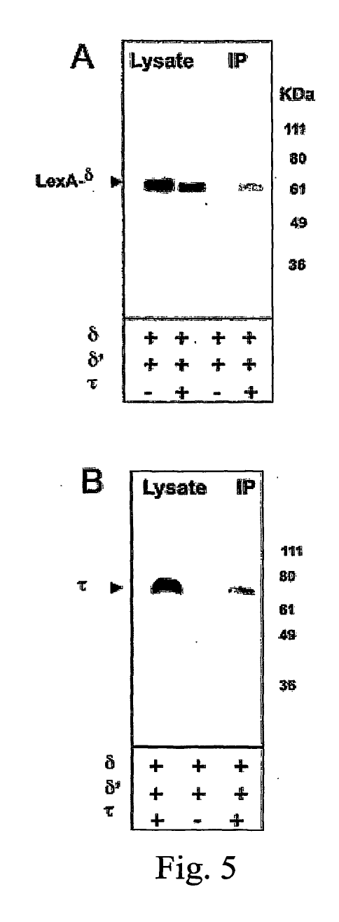

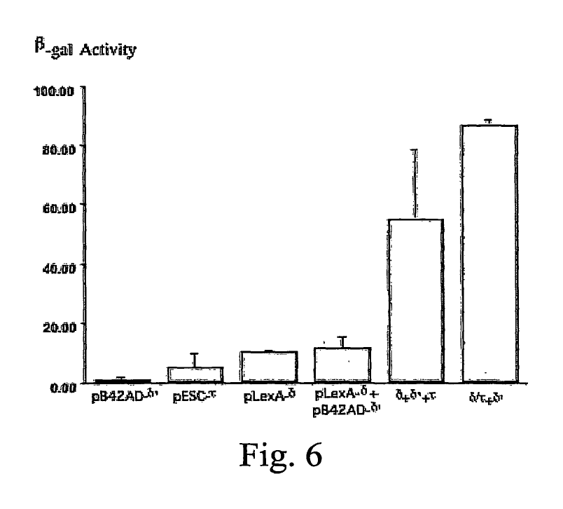

Next we tested the association among H. pylori clamp loading proteins in formation of complex using the yeast two-hybrid system. Each of the three H. pylori clamp loading proteins (δ, δ′ and τ) was expressed as a fusion with either a DNA-binding protein, LexA, or the transcription activation domain of B42. β-galactosidase activity showed no interaction or weak interactions in doubly transformed yeast cells that expressed two types of fusion proteins (FIG. 5). In FIG. 5, EGY40[p8op-lacZ] was transformed with plasmids expressing LexA-δ and B42-δ′ and τ. Protein extracts were prepared from cells grown in 2% galactose in order to induce gene expression. Immunoprecipitations performed with anti-HA (12A5) antibodies. Cell lysates and immunoprecipitates (IP) were analysed on immunoblotted with polyclonal anti-LexA antibody (A); immunoblotted with anti-myc antibody (B). The positions of LexA-δ (predicted molecular mass of 65 kDa) and τ predicted molecular mass of 70 kDa) are indicated by arrows. We reasoned that although the two-hybrid system can detect interaction between two well-defined proteins, this method failed to detect interactions between proteins that are part of a larger protein complex such as the clamp loader studied here. This may be due to the weak interactions which exist between two members of the multi-protein complex. Therefore, we asked whether the presence of τ would enhance δ and δ′ interaction. To test this in yeast cells, we introduced a third plasmid expressing τ into the system. Transformants that simultaneously expressed LexA-δ, B42-δ′ and unfused τ exhibited significantly higher β-galactosidase activity than those producing LexA and B42-δ′ (FIG. 6). In FIG. 6, plasmids were transformed into EGY[p8op-lacZ] in a variety of combinations and assayed for β-Galactosidase activity, expressed in Miller units. Negative control transformants that produced LexA-δ, unfused B42 and τ did not show β-galactosidase activity (results not shown). Similar results obtained when the two proteins LexA-δ and τ were expressed from the same vector (pESCLexAHpδ/τ). We also confirmed that the amount of LexA-δ and B42-δ′ hybrid proteins accumulated were unchanged both in δδ′τ-expressing yeast cells and in δδ′-expressing yeast cells, as estimated by Western blots using anti-HA and anti-LexA antisera (results not shown). Thus the presence of τ is not likely to affect the level of expression of stability of LexA-δ and B42-δ′ proteins. The results show that δ and δ′ can interact in the presence of τ.

Formation of a Clamp Loader (δδ′τ) Complex

Taken together, our results demonstrate that activation of the reporter gene transcription by the reconstituted activator LexA/B42 results from the formation of a LexA-δ-B42-δ′ protein complex which is promoted by a third partner in the clamp loader complex, τ. Such protein complexes can be visualized by immunoprecipitation from whole double transformed yeast cell extracts using antibodies directed towards the HA epitope of the B42-δ′ hybrid protein. Using anti-HA antibodies (12A5), we were able to immunoprecipitate not only LexA-δ but also τ from the yeast total cell extract (FIG. 5).

### EXAMPLE 4

In this example, we identify the δ peptide motif responsible for the interaction of the δ protein with β.

### A. Methods

Analysis of the Amino Acid Sequences of the δ Family

Predicted secondary structures were determined using the PSIPRED and GenThrEADER servers at http://insulin.brunel.ac.uk/psipred and the Jpred server at http://jura.ebi.ac.uk:8888/submit.html. Protein fold recognition was carried out using the 3D_PSSM server v2.5.1 at http://www.bmm.icnet.uk/˜3dpssm. Modelling of δ protein structure based on the β′ structure was undertaken using the SWISS-MODEL server at http://www.expasy.ch/swissmod/SWISS-MODEL.html and viewed using SwissPdbViewer.

Construction of Expression of Plasmids and Mutagenesis.

Plasmids expressing E. coli δ with an N-terminal His₆-tag were constructed in pET20b (Novagen). The LF to AA mutation of His₆-δ was introduced using the site directed mutagenesis method (Quikchange mutagenesis kit, Stratagene) according to the manufacturer's instructions. The mutagenic primers used were:

| | |
|---|---|
| 5′-GCCAGGCTATGAGTGCGGCTGCCAGTCGACAAAC-3′, | (Seq. ID |
| | No. 620) |
| and | |
| 5′-GTTTGTCGACTGGCAGCCGCACTCATAGCCTGGC-3′. | (Seq. ID |
| | No. 621) |

Ni-NTA Co Immobilisation Assay

The in vitro His₆-tagged δ protein was allowed to bind to Ni-NTA resin in 200 μl of binding buffer (50 mM NaH₂PO₄, 300 mM NaCl, 10 mM imidazole, pH8) at 4° C. for 1 h. The Ni-NTA resin was then washed 3 times with wash buffer (50 mM NaH₂PO₄, 300 mM NaCl, 20 mM imidazole pH8). In vitro transcribed-translated [³⁵S]-labelled β protein was added to Ni-NTA resin in BB14 interaction buffer (20 mM Tris pH7.5, 0.1 mM EDTA, 25 mM NaCl and 10 mM MgCl₂) and allowed to bind for 1 h at RT. The resin was then washed 3 times with WB3 buffer (20 mM Tris pH7.5, 0.1 mM EDTA, 0.05% Tween20). The bound proteins eluted by heating the resin for 5 min at 100° C. in SDS-PAGE reducing sample buffer. [³⁵S]-labelled proteins were visualised by autoradiography.

### B. Results

Domain Organisation of δ Family Proteins

During the PSI BLAST searches of the databases a substantial number of the hits of borderline significance with bacterial γ/τ and archeal and eukaryotic clamp loader proteins (RFC subunits) and bacterial DnaA proteins in the region of these proteins that contains the AAA+ domain were registered. The AAA+ domain is involved in ATP-binding and is also proposed to be involved in subunit oligomerisation of many members of the extremely large family of proteins that contain it (Neuwald et al., Genome Research (1999) 9: 27-43). Many of these proteins are associated with the assembly, operation and disassembly of protein complexes (Neuwald et al., 1999). Given the role of δ in the clamp loader these similarities were explored in more detail. On the basis of the alignments produced from the PSI BLAST and HMM searches and the nature of the conservation of residues, representative δ sequences were aligned with the AAA+ domain regions of E. coli δ′ and γ/τ (FIG. 3). The predicted secondary structure of E. coli δ by two different methods is in good agreement with the experimentally determined secondary structure features of E. coli δ′ (FIG. 3). Furthermore, fold-recognition searches using the 3D-pssm fold recognition server with the H. pylori, E. coli and Aquifex aeolicus δ sequences identified matches to the E. coli δ′ structural folds with probabilities of 0.13, 8.01e-07, 5.15e-06 and respectively, providing further support for the proposal that the amino-terminal region of δ folds into an AAA+ domain. T he most conserved residues in the AAA+ family domain are those involved in the ATPase activity. Since δ, like δ′, does not have ATPase activity we would not expect these residues to be conserved. Rather we would expect conservation of residues that contribute to the secondary and tertiary structure of the domain. Good conservation is seen for the core residues of the δ′ structure.

Despite extensive searching no significant relationships were identified between the carboxy-terminal regions of the δ orthologues and the other clamp loading proteins from eubacteria, or with the clamp loading proteins from eukaryotes, archea and bacteriophages, or with any other proteins in the non-redundant protein database at GenBank.

Identification of β-Binding Site in δ

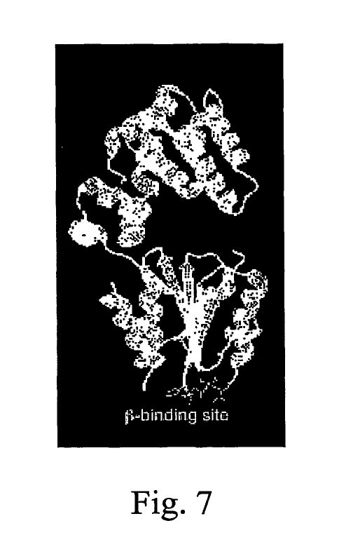

When the positions of the most conserved residues in δ were mapped on our structural model of δ, a phenylalanine conserved in the δ family, but not elsewhere, located in the second half of the Box IV′ preceding the Walker B box (FIG. 3) was identified. It mapped as exposed on a surface loop in a region of δ putatively independent of inter-subunit interactions (FIG. 7). The other conserved amino acids were in regions conserved in δ, γ/τ or another of the clamp loaders (FIG. 3). The conserved phenylalanine is part of a region with the loose consensus sequence sLF[AG] (where s is a small amino acid) (Table 15) and which is a good candidate for a role in the binding of δ to β during the loading of β onto DNA.

TABLE 15

Delta Protein Family Sequences

| | |
|---|---|
| Seq. ID | Sequence |

| No. | Sequence name | N-term | Motif | C-term |
|---|---|---|---|---|
| 1 | 741 delta Aquifex aeolicus VF5 | SEEEFYTALS | ETSIF | GGSKEKAVVI |
| 2 | 740 delta Thermotoga maritima MSB8 | KIDFIRSLLR | TKTIF | SNKTIIDIVN |
| 3 | 1803 delta Chloroflexus aurantiacus J-10-f1 | QLVAACE | AHPFL | AERRLVIVYD |
| 4 | 739 delta Deinococcus radiodurans R1 | VSAETLGPHL | APSLF | GDGGVVVDFE |
| 5 | 738 delta Porphyromonas gingivalis W83 | SVADIANEAR | RFPMM | GRRQLIVVRE |
| 6 | 769 delta Bacteroides fragilis NCTC9343 | DVATVINAAK | RYPMM | SEHQVVIVKE |
| 7 | 751 delta Cytophaga hutchinsonii JGI | NVSTILQNAR | KYPMF | SERQVVMVKE |
| 8 | 737 delta Chlorobium tepidum TLS | TLGQIVSAAS | EYPMF | TEKKLVVVRQ |
| 9 | 736 delta Chlamydia trachomatis | LQQELLSWTD | HFGLF | ASQETIGIYQ |
| 10 | 735 delta Chlamydophila pneumoniae | MPATLMSWTE | TFALF | QEHETLGIIH |
| 11 | 733 delta Nostoc punctiforme ATCC29133 | AAIQALNQVM | TPTFG | AGGRLVWLIN |
| 12 | 755 delta Anabaena sp. PCC7120 | AAIQALNQVM | TPAFG | AGGRLVWLMN |
| 13 | 734 delta Synechocystis sp. PCC6803 | ATQRGLEQAL | TPPFG | SGDRLVWVVD |
| 14 | 732 delta Prochlorococcus marinus MED4 | QIKQAFDEIL | TPPLG | DGSRVVVLKN |
| 15 | 780 delta Prochlorococcus marinus MIT9313 | QASQALAEAR | TPPFG | SGGRLVLLQR |
| 16 | 754 delta Synechococcus sp. WH8102 | QAAQALDEAR | TPPFA | SGERLVLLQR |
| 17 | 1810 delta Treponema denticola TIGR | GMGDVISLLQ | NASLF | SSAKLIILKS |
| 18 | 731 delta Treponema pallidum Nichols | PVADLVDLLR | TRALF | ADAVCVVLYN |
| 19 | 730 delta Borrelia burgdorferi B31 | SAVGFAEKLF | SNSFF | SKKEIFIVYE |
| 20 | 752 delta Magnetospirillum magnetotacticum | IPSRLADEAA | AMALG | GGRRVVVLRD |
| | MS-1 | | | |
| 21 | 753 delta Magnetospirillum magnetotacticum | DPGRLVDEAG | TVGLF | GGSRTIWVRS |
| | MS-1 | | | |
| 22 | 706 delta Rhodopseudomonas palustris CGA009 | EPSRLVDEAL | AIPMF | GGRRAIRVRA |
| 23 | 778 delta Mesorhizobium loti MAFF303099 | DEGRLLDEAR | TVPMF | SDRRLLWVRN |
| 24 | 743 delta Brucella suis 1330 | DPAKLADEAG | TISMF | GGQRLIWIKN |
| 25 | 1808 delta Sinorhizobium meliloti 1021 | GAGSVLDEVN | AIGLF | GGDKLVWVRG |
| 26 | 1809 delta Agrobacterium tumefaciens C58 | DPGRLLDEVN | AIGLF | GGEKLVWVKS |
| 27 | 707 delta Caulobacter crescentus TIGR | DPAKLEDELS | AMSLM | GGRRLVRLRL |
| 28 | 782 delta Rhodobacter sphaeroides 2.4.1 | DPAALMDAMT | AKGFF | EGPRAVLVEE |
| 29 | 1799 delta Rickettsia conorii Malish_7 | NISSLEILLN | SSNFF | GQKELIKIRS |
| 30 | 708 delta Rickettsia prowazekii Madrid_E | NILSLDILLN | SPNFF | GQKELIKVRS |
| 31 | 746 delta Wolbachia sp. TIGR | SPSLLFSELA | NVSMF | TSKKLIKLIN |
| 32 | 702 delta Neisseria gonorrhoeae FA1090 | DWNELLQTAG | NAGLF | ADLKLLELHI |
| 33 | 701 delta Neisseria meningitidis Z2491 | DWNELLQTAG | SAGLF | ADLKLLELHI |
| 34 | 703 delta Nitrosomonas europaea | DWMNLFQWGR | QSSLF | SERRMLDLRI |
| | Schmidt_Stan_Watson | | | |
| 35 | 704 delta Bordetella pertussis Tohama_I | DWSAVAAATQ | SVSLF | GDRRLLELKI |
| 36 | 1807 delta Burkholderia pseudomallei K96243 | DWSTLIGASQ | AMSLF | GERQLVELRI |
| 37 | 748 delta Burkholderia cepacia LB400 | DWSSLLGASQ | SMSLF | GDRQLVELRI |
| 38 | 742 delta Burkholderia mallei ATCC23344 | DWSTLIGASQ | AMSLF | GERQLVELRI |
| 39 | 749 delta Ralstonia metallidurans CH34 | QWGQVIEAQQ | SMSLF | GDRKIVELRI |
| 40 | 699 delta Acidothiobacillus ferrooxidans | IWDALRDERD | AGSLF | AAQRVLLLRL |
| | ATCC23270 | | | |
| 41 | 700 delta Xylella fastidiosa | DWQQLASSFN | APSLF | SSRRLIEIRL |
| | 8.1.b_clone_9.a.5.c | | | |
| 42 | 698 delta Legionella pneumophila | EWHVVLEETN | NYSLF | YQTVILTIFF |
| | Philadelphia-1 | | | |
| 43 | 744 delta Coxiella burnetii | HWQSLTQSFD | NFSLL | SDKTLIELRN |
| | Nine_Mile_(RSA_493) | | | |
| 44 | 745 delta Methylococcus capsulatus TIGR | SWSTFLEAGD | SVPLF | GDRRILDLRL |
| 45 | 696 delta Pseudomonas aeruginosa PAO1 | DWGLLLEAGA | SLSLF | AEKRLIELRL |
| 46 | 697 delta Pseudomonas putida KT2440 | DWGTLLQAGA | SLSLF | AQRRLLELRL |
| 47 | 759 delta Pseudomonas syringae DC3000 | DWGTLLQAGA | SMSLF | AERRLLELRL |
| 48 | 750 delta Pseudomonas fluorescens Pf0-1 | DWGTLLQAGA | SMSLF | AEKRLLELRL |
| 49 | 695 delta Shewanella putrefaciens MR-1 | NWGDLTQEWQ | AMSLF | SSRRIIELTL |
| 50 | 694 delta Vibrio cholerae N16961 | DWNAVYDCCQ | ALSLF | SSRQLIEIEI |
| 51 | 690 delta Pasteurella multocida Pm70 | NWSDLFERCQ | SIGLF | FNKQILFLNL |
| 52 | 691 delta Haemophilus influenzae KW20 | DWAQLIESCQ | SIGLF | FSKQILSLNL |
| 53 | 692 delta Haemophilus ducreyi 35000HP | KWEQLFESVQ | NFGLF | FSRQIIILNL |
| 54 | 693 delta Actinobacillus | DWNDLFERVQ | SMGLF | FNKQLIILDL |
| | actinomycetemcomitans HK1651 | | | |
| 55 | 689 delta Buchnera sp. APS | DWKKIILFYK | TNNLF | FKKTTLVINF |
| 56 | 685 delta Escherichia coli MG1655 | DWNAIFSLCQ | AMSLF | ASRQTLLLLL |
| 57 | 686 delta Salmonella typhi CT18 | DWGSLFSLCQ | AMSLF | ASRQTLVLQL |
| 58 | 764 delta Salmonella typhimurium | DWGSLFSLCQ | AMSLF | ASRQTLVLQL |
| 59 | 687 delta Klebsiella pneumoniae MGH78578 | PTGRRFSLKP | GDELF | ASRQTLLLIL |
| 60 | 688 delta Yersinia pestis CO-92 | EWEHIFSLCQ | ALSLF | ASRQTLLLSF |
| 61 | 763 delta Yersinia pseudotuberculosis | EWEHIFSLCQ | ALSLF | ASRQTLLLSF |
| | IP32953 | | | |
| 62 | 766 delta Desulfovibrio vulgaris | LPPVFWEHLT | LQGLF | GSPRALVVRN |
| | Hildenborough | | | |
| 63 | 761 delta Geobacter sulfurreducens TIGR | KGDDIATAAQ | TLPMF | ADRRMVLVKR |
| 64 | 710 delta Helicobacter pylori | EKSQIATLLE | QDSLF | GGSSLVILKL |
| 65 | 709 delta Campylobacter jejuni NCTC11168 | NFTRASDFLS | AGSLF | SEKKLLEIKT |
| 66 | 711 delta Streptomyces coelicolor A3 (2) | LQPGTLAELT | SPSLF | AERKVVVVRN |
| 67 | 767 delta Thermobifida fusca YX | VSAGKLVEVT | SPSLF | GDRRVVVLRS |
| 68 | 713 delta Mycobacterium avium 104 | VSTYELAELL | SPSLF | AEERIVVLEA |
| 69 | 714 delta Mycobacterium leprae TN | VGTYELTELL | SPSLF | ADERIVVLEA |
| 70 | 762 delta Mycobacterium smegmatis MC2_155 | VSTSELAELL | SPSLF | AEERLVVLEA |
| 71 | 712 delta Mycobacterium tuberculosis H37Rv | VGAYELAELL | SPSLF | AEERIVVLGA |
| 72 | 715 delta Corynebacterium diptheriae | VNASELIQLT | SPSLF | GEDRIIVLTN |
| | NCTC13129 | | | |
| 73 | 716 delta Dehalococcoides ethenogenes TIGR | TAAELQNYVQ | TIPFL | APARLVMVNG |
| 74 | 1806 delta Clostridium difficile 630 | VLNHLISSIE | TLPFM | DDRKI |
| 75 | 758 delta Carboxydothermus hydrogenoformans | LPEEVVARAE | TVSFF | GQRFIVVKNC |
| | TIGR | | | |
| 76 | 721 delta Bacillus halodurans C-125 | PIEAALEEAE | TVPFF | GSKRVVILKD |
| 77 | 717 delta Bacillus stearothermophilus 10 | PIEAALEEAE | TVPFF | GERRVILIKH |
| 78 | 718 delta Bacillus subtilis 168 | PLDQAIADAE | TFPFM | GERRLVIVKN |
| 79 | 719 delta Staphylococcus aureus COL | EIAPIVEETL | TLPFF | SDKKAILVKN |
| 80 | 760 delta Staphylococcus epidermidis RP62A | DLTPIIEETL | TMPFF | SNKKAIVVKN |
| 81 | 720 delta Bacillus anthracis Ames | YLEDVVEDAR | TLPFF | GERKVLLIKS |
| 82 | 1800 delta Listeria innocua Clip11262 | PIEVVIQEAE | SMPFF | GDKRLVMANN |
| 83 | 1802 delta Listeria monocytogenes 4b | PIEVVIQEAE | SMPFF | GDKRLVMANN |
| 84 | 1801 delta Listeria monocytogenes EGD-e | PIEVVVQEAE | SMPFF | GDKRLVMANN |
| 85 | 722 delta Enterococcus faecalis V583 | PLSAAIAEAE | TIPFF | GDYRLVFVEN |
| 86 | 756 delta Enterococcus faecium DOE | SLDEVVAEAE | TLPFF | GDQRLVFVEN |
| 87 | 765 delta Lactococcus lactis IL1403 | NSDLALEDLE | SLPFF | SDSRLVILEN |
| 88 | 757 delta Streptococcus equi Sanger | LYQTAEMDLV | SMPFF | ADQKVVIFDH |
| 89 | 723 delta Streptococcus agalactiae | DYQNAELDLE | SLPFL | SDYKVVIFDQ |
| 90 | 724 delta Streptococcus pyogenes M1_GAS | AYQDAEMDLV | SLPFF | AEQKVVIFDH |
| 91 | 747 delta Streptococcus mutans UA159 | SYQDAEMDLE | SLPFF | ADEKIVIFDN |
| 92 | 1804 delta Streptococcus gordonii | DYQQVELDLV | SLPFF | SDEKIIILDH |
| 93 | 725 delta Streptococcus pneumoniae type_4 | VYKDVELELV | SLPFF | ADEKIVILDY |
| 94 | 726 delta Ureaplasma urealyticum Serovar_3 | SLISFKNLIE | QDDLF | NSNKIYLFKN |
| 95 | 728 delta Mycoplasma genitalium G-37 | KDLKQLYDLF | SQPLF | GSNNEKFIVN |
| 96 | 727 delta Mycoplasma pneumoniae M129 | DVNKLYDVVL | NQNLF | AEDTKPILIH |
| 97 | 1805 delta Mycoplasma pulmonis | EIDDLLNDIV | QKDLF | SPNKIIHIKN |
| 98 | 729 delta Clostridium acetobutylicum | EFEDILNACE | TVPFM | SEKRMVVVYR |
| | ATCC824D | | | |

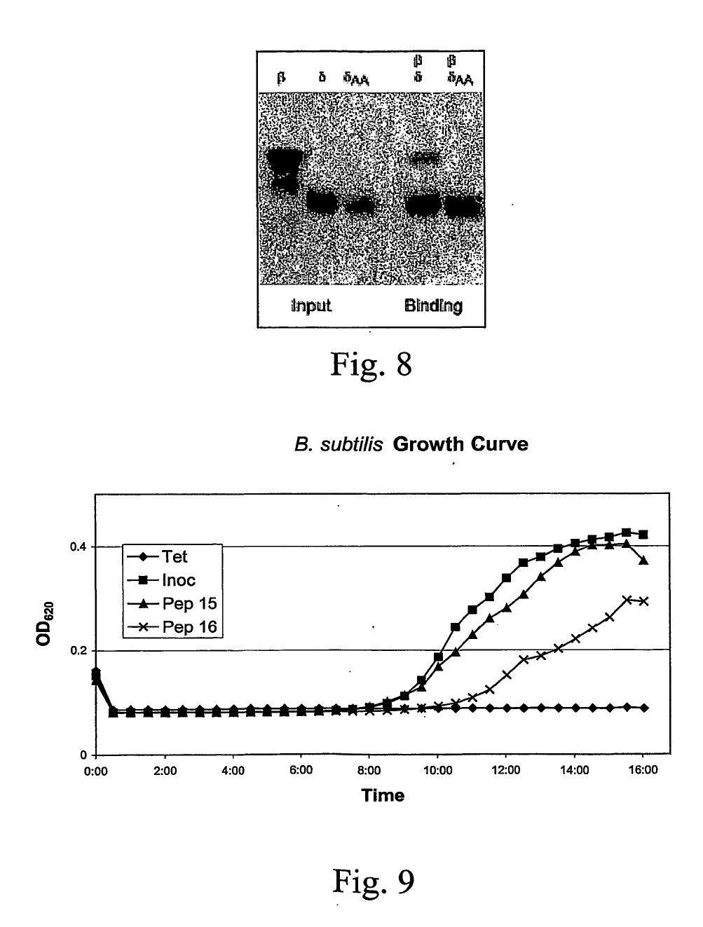

To determine whether the proposed LF peptide motif constitutes part of the β binding site, mutant δ was made by substituting LF with AA (2 alanine). When the AA mutant protein was used in Ni-NTA co immobilisation assay, it did not bind to β (FIG. 8). In FIG. 8, aliquots of 5-15 μl of in vitro transcribed and translated β protein was allowed to bind to immobilized His₆-tagged wild type δ or mutant δ (δ_{AA}). The bound proteins were eluted and applied to SDS-PAGE; 5 μl of input proteins shown in the figure. E. coli, δ-β interaction was clearly disrupted by altering the LF to AA, further demonstrating the importance of this motif for interaction with β (FIG. 8).

### EXAMPLE 5

In this example, we present a model for the binding of the peptide motif identified and characterised in the above examples to eubacterial δ proteins.

### A. Methods

The 3D structure of a subunit of PCNA from PDB coordinate file 1AXC and a subunit of β from PDB coordinate file 2POL from the RCSB Protein Data Bank (http://www.rcsb.org/pdb/index.html) were superimposed using Deep View (http://www.expasy.ch/spdbv/mainpage.htm). The coordinates of the p21 peptide binding to the chosen subunit of PCNA were then merged with the coordinates of β to create a coordinate file containing the coordinates of a subunit of β and of the p21 peptide. The coordinates of amino acids 144 to 148 of the p21 peptide were retained and the rest removed. The five amino acids remaining were mutated to give the peptide QLSLF (Seq. ID No. 622) and the coordinates resaved. These coordinates were the starting point for sixty energy minimisation runs using the flexible docking mode in the InsightII package (Accelrys). The final minimized structures were compared and the five lowest energy structures with the position of the amino-terminal glutamine in a similar position to the starting structure were chosen for further analysis.

### B. Results

Modelling Binding of QLSLF Peptide to β

Mutations in the carboxy-terminus of E. coli β have been shown to reduce the binding of δ to β (Naktinis et al, Cell (1996) 84: 137-145). The nature of the conserved β-binding motifs demonstrated that the major interactions between the β-binding peptide and β where hydrophobic in nature. The structure of β has been determined and deposited in the Protein Database with the code 2POL (Kong et al., Cell (1992) 69: 425-437). The region of the surface of β in the vicinity of the carboxyl-terminus was analysed for hydrophobic areas. Two such pockets were identified. The amino acids contributing to the two pockets in all of the available sequences of eubacterial β proteins are listed in Table 16.

TABLE 16
Phylogenetic variation in the residues proposed to contribute to the hydrophobic pockets
on β to which the β-binding peptide binds

| | Position (numbered according to E. coli sequence) | | | | | | | | | |
|---|---|---|---|---|---|---|---|---|---|---|
| Species | 170 | 172 | 175 | 177 | 241 | 242 | 247 | 346 | 360 | 362 |
| Escherichia coli | V | T | H | L | F | P | V | S | V | M |
| Salmonella typhi | V | T | H | L | F | P | V | S | V | M |
| Salmonella typhimurium | V | T | H | L | F | P | V | S | V | M |
| Yersinia pestis | V | T | H | L | F | P | V | S | V | M |
| Proteus mirabilis | V | T | H | L | F | P | V | S | V | M |
| Buchnera aphidicola 1 | V | T | Y | L | Y | P | V | S | V | M |
| Buchnera aphidicola 2 | V | T | Y | L | Y | P | I | S | V | M |
| Buchnera aphidicola 3 | V | T | Y | L | Y | P | V | S | V | M |
| Buchnera aphidicola 4 | V | T | Y | L | Y | P | I | S | V | M |
| Buchnera aphidicola 5 | V | T | Y | L | Y | P | I | S | V | M |
| Pasteurella multocida | V | T | H | L | F | P | V | S | V | M |
| Haemophilus influenzae | V | T | H | L | F | P | V | S | V | M |
| Vibrio cholerae | V | T | H | M | F | P | V | S | V | M |
| Shewanella putrefaciens | I | T | H | L | F | P | V | S | V | M |
| Pseudomonas aeruginosa | V | T | H | L | F | P | V | S | V | M |
| Pseudomonas putida | V | T | H | L | F | P | V | S | V | M |
| Legionella pneumophila | V | T | H | M | F | P | A | S | I | M |
| Thiobacillus ferroxidans | V | T | H | L | Y | P | V | S | I | M |
| Neisseria gonorrheae | V | T | H | L | F | P | V | S | I | M |
| Neisseria meningiditis | V | T | H | L | F | P | V | S | I | M |
| Nitrosomonas europea | V | T | H | L | F | L | A | S | V | M |
| Bordetella bronchiseptica | V | T | H | L | F | P | V | S | V | M |
| Bordetella pertusis | V | T | H | L | F | P | V | S | V | M |
| Rickettsia prowazekii | A | T | Y | L | F | P | F | S | V | M |
| Caulobacter crescentus | V | T | H | L | F | P | V | P | V | M |
| Campylobacter jejuni | V | T | K | L | F | P | V | A | I | M |
| Helicobacter pyloris J99 | V | T | K | L | Y | P | I | P | L | M |
| Helicobacter pylori 26695 | V | T | K | L | Y | P | I | P | L | M |
| Streptomyces coelicolor | A | T | Y | F | L | P | L | P | L | M |
| Mycobacterium avium | A | T | F | L | F | P | L | P | L | M |
| Mycobacterium bovis | A | T | F | L | F | P | L | P | L | M |
| Mycobacterium leprae | A | T | F | L | F | P | L | P | L | M |
| Mycobacterium smegmatis | A | T | F | L | F | P | L | P | L | M |
| Bacillus subtilis | T | T | H | L | Y | P | L | P | L | L |
| Staphylococcus aureus | T | T | H | L | Y | P | L | P | L | L |
| Bacillus anthracis | I | T | H | L | Y | P | L | P | L | L |
| Bacillus halodurans | T | T | H | L | Y | P | M | P | L | S |
| Lactococcus lactis | V | T | H | M | Y | P | L | P | L | T |
| Streptococcus pyogenes | V | T | H | M | Y | P | L | P | L | T |
| Streptococcus mutans | V | T | H | M | Y | P | L | P | L | T |
| Streptococcus pneumoniae | V | T | H | L | Y | P | L | P | L | T |
| Streptococcus pneumoniae 2 | V | T | H | L | Y | P | L | P | L | T |
| Mycoplasma capricolum | S | T | F | I | F | P | A | P | V | L |
| Spiroplasma citri | T | T | F | L | Y | P | V | P | L | L |
| Ureaplasma urealyticum | I | T | I | A | Y | P | I | P | I | S |
| Mycoplasma genitalium | E | S | Y | L | F | P | F | Y | I | V |
| Mycoplasma pneumoniae | E | S | Y | L | F | P | L | Y | I | V |
| Clostridium acetobutylicum | V | I | Y | L | F | I | I | P | L | L |
| Treponema pallidum | V | T | K | L | F | P | V | A | I | M |
| Borrelia burgdorferi | V | T | H | M | Y | P | I | K | L | M |
| Synechocystis PCC7942 | A | T | H | L | Y | P | L | P | L | M |
| Synechocystis sp | A | T | H | L | Y | P | L | P | L | M |
| Prochlorococcus marinus | A | T | H | L | Y | P | L | P | L | M |
| Chlamydophila pneumoniae | V | T | K | L | F | P | V | P | V | M |
| Chlamydia pneumoniae AR39 | V | T | K | L | F | P | V | P | V | M |
| Chlamydia trachomatis | V | T | K | L | F | P | V | P | V | M |
| Chlamydia muridarum | V | T | K | L | F | P | V | P | V | M |
| Chlorobium tepidum | V | T | H | L | Y | P | V | A | L | M |
| Porphyromonas gingivalis | V | S | Q | L | Y | P | V | A | L | L |
| Deinococcus radiodurans | V | S | Y | V | F | P | V | P | L | R |
| Thermotoga maritima | V | S | R | L | F | P | V | P | I | M |
| Aquifex aeolicus | V | S | H | L | F | P | V | A | I | M |

Modelling of the QLSLF (Seq. ID No. 622) consensus peptide into this region indicated that these amino acids were likely to contribute to the binding of the β-binding peptides to β. Therefore these amino acids constitute that part of the surface of β which interacts with the β-binding peptides.

### EXAMPLE 6

A number of peptide analogues of the β protein-binding motif were tested for their ability to inhibit the binding of the replisomal proteins α and δ to β. The results of these experiments follow.

### A. Methods

Plate Inhibition Assays

Recombinantly expressed wild type E. coli α subunit was purified and coated onto 96 well microtitre plates (Falcon flexible plates, Becton Dickinson) at 20 μg/ml in 100 mM Na₂CO₃, pH9.5 (50 μl/well, 4° C. overnight or 2 h, RT (RT). The plates were washed in WB3 (20 mM Tris (pH 7.5), 0.1 mM EDTA containing 0.05% v/v Tween 20). This buffer was used in all wash steps through out the assay. The plates were then blocked with “blotto” (5% skim milk powder in WB3, 100 μl/well, RT) until required. Immediately before use the plates were washed.

The purified synthetic peptides and β subunit were diluted in BB14 (20 mM Tris, pH 7.5, 10 mM MgCl₂, 0.1 mM EDTA). Purified synthetic peptides with concentrations of 9.3-300 and 1000 μg/ml were allowed to complex with purified wild type β subunit (5 μg/ml) in a 96 well microtitre plate (Sarsted, Adelaide, Australia) pre-treated with “blotto” (30 min, RT). The reaction volume was 120 μl. The β subunit also was incubated in the absence of peptide or in the presence of the α subunit at 76.5 (μg/ml in BB14. All samples were incubated for 1 h (RT). Two 50 μl samples were transferred from each well to a corresponding well of the washed and “blocked” α subunit coated plates, and further incubated for 30 min (RT).

The plates were washed and treated with rabbit serum raised to the β subunit. The anti-serum was diluted 1:1000 in WB3 containing 10% “blotto”, dispensed at 50 μl/well and incubated for 12 min (RT). The plates were washed again and treated with sheep anti-rabbit Ig-HRP conjugate (Silenus, Melbourne, Australia) diluted 1:1000 in WB3 containing 10% “blotto” (50 μl/well). The plate was incubated for 12 min (RT). After a final washing step, 1 mM 2,2′-azino-bis (3-ethylbenzthiazoline-6-sulfonic acid) was added (110 μl/well). Colour development was assessed at 405 nm using a plate reader (Multiskan Ascent, Labsystems, Sweden).

The δ-β plate binding assay followed a similar regime but with the following changes: purified wild-type E. coli δ subunit was coated onto the plate at 5 μg/ml; the same concentration of synthetic peptides were preincubated with the β subunit at 1 μg/ml; and the pre-formed peptide-complexes were transferred to the δ subunit coated plates and incubated for only 10 min.

### B. Results

Several nine amino acid peptides with sequences based on the amino acid sequence containing the QxSLF motif in DnaE were synthesised and purified. The peptides and their sequences are listed in Table 17.

TABLE 17
Results of peptide inhibition assays

| | |
|---|---|
| Seq. ID | IC₅₀ μg/ml |

| | | | | |
|---|---|---|---|---|
| Peptide | No. | Sequence | α | δ |

| | | | | |
|---|---|---|---|---|
| DnaE | 640 | IG QADMF GV | 14.6 | 218 |
| pep1 | 641 | IG QLDMF GV | 2.8 | 12.9 |
| pep2 | 642 | IG QASMF GV | 860 | ni^{a} |
| pep3 | 643 | IG QADAF GV | ni | ni |
| pep4 | 644 | IG QADMA GV | ni | ni |
| pep5 | 645 | IG QAVMF GV | nd^{b} | ni |
| pep6 | 646 | IG PADMF GV | ni | ni |
| pep7 | 647 | IG KADMF GV | ni | ni |
| pep8 | 648 | IG QADKF GV | ni | ni |
| pep9 | 649 | IG QADMK GV | ni | ni |
| pep11 | 650 | IG QAAMF GV | ni | ni |
| pep12 | 651 | IG AADMF GV | ni | ni |
| pep13 | 652 | IG QLSLF GV | 1.42 | 9.5 |
| pep14 | 653 | IG QLDLF GV | 1.33 | 8.8 |
| pep15 | | QLD | ni | ni |
| pep16 | | DLF | 135 | 1200 |

^{a}—no inhibition;
^{b}—not done

Five nonapeptides, DnaE, and peptides 1, 2, 13, and 14 produced significant inhibition of the binding of α to β (Table 17). The sequence related nonapeptides 3 to 12 did not cause any inhibition of α:β binding. Peptides 1, 13, 14 and DnaE also inhibited the binding of δ to β. (Table 17). All other nonapeptides did not significantly inhibit β binding.

Peptide Assays

We have demonstrated that specific peptides of nine amino acids can bind to β and prevent binding of both αand δ to β, thus confining the limited extent of the residues required for interaction with β. These results also validate the assays for use in the screening for compounds that interfere with the binding of α and/or δ to β, by providing further evidence that the interactions being assayed are likely to be similar to if not identical to the interactions in cells.

### EXAMPLE 7

Design of a Tripeptide Inhibitor of α:β and δ:β Protein-Protein Interactions.

In order to design smaller inhibitors of the interaction between proteins containing the β-binding peptides and β, the variation in the sequences of the β-binding peptides and the binding inhibition assay data was examined in detail. The highest level of conservation observed was for the amino acids in positions one, four and five (FIG. 9).

More than 70% of the peptide sequences (excluding δ) contained leucine in position four and phenylalanine in position five. The high level of conservation of the LF motif showed that these amino acids are major determinants of the interactions between β-binding proteins and β. The mutagenesis and peptide inhibition experiments confirm the importance of the LF motif with the following importance of conforming to the consensus, position 5=4>1>3>2. However, positions 2 and 3 modulate the interaction of the peptides with β. Substitution of the alanine at position two with leucine to generate peptide 2 substantially improves competitiveness, whilst substitution of the aspartic acid at position three with serine, to generate peptide 2 substantially decreased the competitiveness of the peptide. These results predicted that the tripeptide DLF would inhibit binding of α and δ to β, but the tripeptide QLD although containing favoured amino acids was unlikely to inhibit binding. The two tripeptides QLD and DLF were synthesised and purified. As predicted DLF, inhibited α:β binding (Table 17) with 50% inhibition at approximately 135 μg/ml and δ:β binding with 50% inhibition at approximately 1200 μg/ml.

These observations indicate that the dipeptide LF and/or variants thereof (such as MF and DLF) with additional substitutions in the region of the backbone are lead compounds for the design of other compounds able to disrupt the interaction between β-binding proteins and β.

### EXAMPLE 8

In this example, we demonstrate that the tripeptide DLF, an in vitro inhibitor of α:β and δ:β interactions, inhibits the growth of Bacillus subtilis.

### A. Methods

B. subtilis IH 6140 was subcultured from a fresh plate into a 10 ml tube containing 5 ml of Oxoid Mueller-Hinton broth (Oxoid code CM405 Oxoid Manual 7^{th} edition 1995 pg 2-161). This culture was shaken at 120 rpm at 37° C. for 21 h and then diluted in normal saline to 0.5 McFarland Standard (NCCLS Performance standard for Dilution Antimicrobial Susceptibility Testing M7-A4 January 1997). This suspension was further diluted 1:5 in normal saline to form the bacterial starter culture. Peptides were tested at a final concentration of 1 mg/ml in a flat bottom 96 well plate (Nunclon surface, sterile Nalge Nunc International). Wells were prepared by using 100 μl of double strength Mueller-Hinton Broth, an appropriate volume of peptide and the final volume made up to 190 μl. The wells were then inoculated with 10 μl of the starter culture.

The plate was sealed with a clear adhesive plate seal (Abgene House). It was then placed in a Labsystems Multiskan Ascent spectrophotometer. The plate was incubated at 37° C. with shaking at 120 rpm every alternate 10 seconds. The absorbence at 620 nm was measured every 30 min for 16 h.

### B. Results

The tripeptide DLF significantly inhibits the growth of B. subtilis, primarily by increasing the lag phase but also by decreasing the growth rate during the following log phase (FIG. 10). In FIG. 10, the effect of tripeptides on the growth of B. subtilis is graphed as OD₆₂₀ against time of incubation. In contrast, the tripeptide QLD, which did not inhibit the interaction of α and δ with β, did not increase the lag phase but did decrease the growth rate during the log phase (see FIG. 10 and Table 18).

TABLE 18
Effect of DLF on growth of B. subtilis

| | Increase in | Doubling time |
|---|---|---|
| | lag phase | log phase |
| Addition | (Min) | (Min) |
| None | — | 125 |
| QLD | — | 151 |
| DLF | 120 | 187 |

### EXAMPLE 9

In this example we directly demonstrate, by surface plasmon resonance (SPR), the binding of peptides to β protein.

### A. Methods

Surface Plasmon Resonance

Reverse phase HPLC purified peptides (10 μg) were reacted with 1 mg biotin-linker (6-(6-((biotinoyl)amino(hexanoyl) amino) hexanoic acid) sulphosuccinimidyl ester; Molecular Probes, Eugene, Oreg.) (20 mg/ml in DMSO) in 75 mM sodium borate (pH8.5) overnight (RT) with rotation. The reaction mixture was separated using a Brownlee C18 cartridge (Applied Biosystems Inc., Foster City, Calif.) and a gradient of 6-65% acetonitrile in 0.1% TFA delivered at 0.5 ml/min over 40 min by HPLC (Shimadzu, Japan). Biotinylated peptides that eluted later than the biotin-linker and free peptide, were collected, vacuum dried and then dissolved in water. SPR was conducted on a Biacore 2000 using streptavidin derivitised flow cell surfaces (Biacore). All β subunit and free peptide solutions were prepared in BB14 with 150 mM NaCl.

For the KD studies, the biotinylated peptides were loaded onto the flow cell surfaces such that interaction with 0.5 μM β subunit produced a response of 50-100 RU. Upon completion of injection, RU values quickly returned to baseline at 10 and 50 μl/min flow rates, therefore regeneration buffers were not required. The dissociation rates (KD) were determined using the RU values obtained at steady state for 15 different concentrations of the β subunit over 10 nM to 5 μM (in duplicate) for each biotinylated peptide attached to the flow cell surface. The data was fitted to the 1:1 Langmuir model by the BioEvaluation software (Biacore).

For the solution affinity analyses, higher loadings of the biotinylated peptides on the flow cell surfaces, and therefore high RU (700-1000), were established. Loading with peptide 4 generated a negative control surface. Since this peptide does not interact with the β subunit, and RU values on interaction with solutions of β subunit cannot be obtained, the flow cell surface was loaded with the same molar amount of biotinylated peptide 4 as the maximum required for any other biotinylated peptide. In all data manipulations, the RU values of this surface was subtracted from the RU values of the test surface. A calibration curve of RU values generated at different concentrations of the β subunit over 10-100 nM was developed for each biotinylated peptide attached to the flow cell surface. To determine the inhibitory effect of free peptide, 100 nM β subunit was pre-incubated for 5 min with different concentrations of free peptide (10 nM to 4.5 μM, in duplicate) to form a complex of β subunit and peptide and then passed over the flow cell surfaces. The amount of free uncomplexed βremaining was determined from the calibration curve. The log of the concentration of the uncomplexed (free) β subunit was plotted against the log concentration of inhibitory peptide. From these plots, the IC₅₀ value, which in this case is the concentration of peptide required to complex 50 nM β subunit, was determined.

### B. Results

Binding curves exhibited rapid off- and on-rates, the latter too fast to determine by SPR. The KD was determined by fitting data to the 1:1 Langmuir model (Table 19). As anticipated from previous binding experiments, the DnaE peptide returned the highest KD, 2.7 μM, whereas peptide 1 returned the lowest KD, 500 nM. Peptides 13 and 14 gave very similar values, 778 and 800 nM, respectively.

To further differentiate the peptides, the IC₅₀ values of peptides 1, 4, 13 and 14 were determined in competition with biotinylated peptides 1, 4 and 14 attached to flow cell surface by solution affinity analysis. The peptide 4 surface was used as a negative control. The IC₅₀ values for each peptide competing against biotinylated peptides 1 and 14 attached to the flow cell surface are listed in Table 19.

TABLE 19
Summary of kinetic parameters obtained by SPR

IC₅₀

| Peptide | KD | β-peptide 1¹ | β-peptide 14 |
|---|---|---|---|
| DnaE peptide | 2.7 μM | n.d.² | n.d. |
| Peptide 1 | 558 nM | 920 nM | 1.01 μM |
| Peptide 4 | n.d. | >>10 μM | >>10 μM |
| Peptide 13 | 800 nM | 440 nM | 550 nM |
| Peptide 14 | 778 nM | 400 nM | 500 nM |

¹b-peptide: biotinylated peptide on flow cell surface
²n.d.: not done

The results presented in Table 19 indicate that peptides 13 and 14 are better competitors for the β subunit in solution than peptide 1, and that peptide 14 is slightly better than peptide 13.

### EXAMPLE 10

In this example we alter the structure of a peptide and assay for inhibition of binding of α to β, demonstrating that some modifications of the peptide do not alter activity.

### A. Methods

A peptide with modified amino and carboxy-termini was synthesized and assayed for its ability to inhibit the interaction of α with β. The peptide was synthesised and assayed as described in Example 6.

### B. Results

The results presented in Table 20 show that acetylation of the amino-terminus and amidation of the carboxy-terminus of DLF had no significant impact on its ability to inhibit binding of α to β (compare the results for peptides 16 and 18).

TABLE 20

| Peptide | Sequence | IC₅₀ α:β (μM) |
|---|---|---|
| pep16 | DLF | 135 |
| pep18 | Ac-DLF-NH₂ | 135 |

### EXAMPLE 11

In this example we use the modelled structures of QLSLF (Seq. ID No. 622) bound to β, derived in Example 5, and the experimental results from Example 6 as the basis for virtual screening of libraries of chemicals. The example demonstrates a method for identification of mimetics of components of the β-binding peptides based on the sequence information derived from the bioinformatics and experimental analysis.

### A. Methods

The structures of QLSLF (Seq. ID No. 622) and the substructures SLF and LF extracted from the results of the modelling were used to search the NCI (National Cancer Institute) compound database (http://129.43.27.140/ncidb2/) using the “simple screen test” and various levels of “tanimoto index” options of the similarity search. In addition, DLF generated by mutating the S to D in QLSLF (Seq. ID No. 622) using the following site was also used:

Deep View (http://www.expasy.ch/spdbv/mainpage.htm).

### B. Results

A number of compounds were identified in each of these screens. Representative compounds are included in the tables referred to in Examples 13 and 14 below.

### EXAMPLE 12

In this example we used the consensus sequence of β-binding peptides, derived in Example 1 and the experimental results from Example 6 as the basis for virtual screening of chemical libraries. The example demonstrates a second method for identification of mimetics of components of the β-binding peptides based on the sequence information derived from the bioinformatics and experimental analysis.

### A. Methods

The sequences SLF and DLF were used to search the PDB database for the occurrence of these sequences in proteins with determined 3D structures. The substructures were removed from the files and superimposed to generate pharmacophore models of SLF and DLF using components of the Tripos suite of Cheminformatics programs (Tripos Inc.). The pharmacophore models were then used to search the NCI and CMS (CSIRO Molecular Science) libraries of compounds.

### B. Results

As in the previous example, a number of compounds were identified in each of these screens. Representative compounds are included in the tables referred to in Examples 13 and 14 below.

### EXAMPLE 13

In this example, we present the results of the testing of a number of the chemical compounds identified in Examples 11 and 12 for their ability to inhibit the interaction of α and δ with β and demonstrate that some chemical mimetics of components of the β-binding peptides do inhibit the interactions.

### A. Methods

Compounds with high similarity scores, or at the intersection of the results of searches using a number of different approaches, and available from the NCI or CMS libraries were obtained and screened as described in Example 6. For the CMS compounds in the of α:β assays, buffer BB37 replaced buffer BB14. Buffer BB37 contains 10 mM MnCl₂ instead of the 10 mM MgCl₂ used in BB14. The buffer conditions were changed to improve the reproducibility and sensitivity of the α:β binding assay.

### B. Results

Eleven NCI compounds and twenty CMS compounds were screened for their ability to inhibit the interaction of α and δ with β. Three compounds with significant inhibition of either of the two binding assays were identified. One of the compounds, 131123, significantly inhibited the interaction of α with β, and two, 33850 and AOC-07877 significantly inhibited the interaction of δ with β (see Table 21 below). Thus, chemical mimetics of components of the β-binding peptides can inhibit the binding of E. Coli α and δ to E. coli β. The compounds have the following structures:

TABLE 21

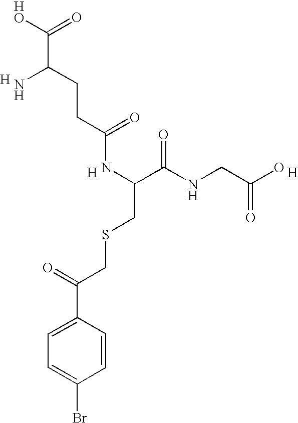

131123

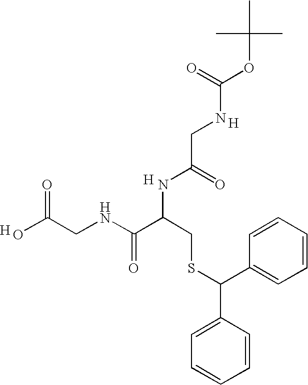

338500

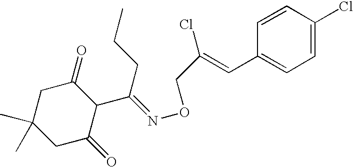

AOC-07877
Results of Chemical Compound Screen

| Compound | Origin | IC₅₀ α-binding (μM) | IC₅₀ δ-binding (μM) |
|---|---|---|---|
| 23336 | NCI | Insoluble | insoluble |
| 125176 | NCI | Partially insoluble | Partially insoluble |
| 131115 | NCI | >1000 | >1000 |
| 131123 | NCI | 210 | >1000 |
| 131127 | NCI | >1000 | >1000 |
| 163356 | NCI | >1000 | >1000 |
| 338500 | NCI | >1000 | 146 |
| 343030 | NCI | >1000 | >1000 |
| 350589 | NCI | >1000 | >1000 |
| 353484 | NCI | >1000 | >1000 |
| 400883 | NCI | >1000 | >1000 |
| AOC-04852 | Molsci | >300 | >300 |
| AOC-05646 | Molsci | >300 | inf |
| AOC-05159 | Molsci | >300 | >300 |
| AOC-06097 | Molsci | >300 | inf |
| AOC-06099 | Molsci | >300 | >300 |
| AOC-06240 | Molsci | >300 | >300 |
| AOC-07182 | Molsci | >300 | >300 |
| AOC-05020 | Molsci | >300 | inf |
| AOC-07499 | Molsci | >300 | inf |
| AOC-07877 | Molsci | 270 | 90 |
| AOC-08944 | Molsci | >300 | >300 |
| DCP-31462 | Molsci | 800 | >1000 |
| DCP-31461 | Molsci | 300 | 560 |
| DCP-31458 | Molsci | 365 | 500 |
| DCP-31451 | Molsci | >1000 | >1000 |
| DCP-31448 | Molsci | >1000 | >1000 |
| DCP-31452 | Molsci | >1000 | >1000 |
| DCP-31446 | Molsci | >1000 | 560 |
| DCP-31444 | Molsci | >1000 | 650 |
| AOC-05203 | Molsci | 365 | 310 |

### EXAMPLE 14

In this example we illustrate the screening of a number of the chemical mimetics identified in Examples 11 and 12 of components of the β-binding peptides for their ability to inhibit the growth of bacteria.

### A. Methods

Compounds with high similarity scores, or at the intersection of the results of searches using a number of different approaches, and available from the NCI or Molecular Science libraries were obtained and screened for inhibition of growth of E. coli ATCC 35218, Klebsiella pneumoniae ATCC 13885, Pseudomonas aeruginosa ATCC 27853, Staphylococcus aureus ATCC 25923 and Enterococcus faecalis ATCC 33186 as follows. Compounds were supplied dissolved in DMSO at 1 mg/ml in a 96 well tray format. Six corresponding slave plates were prepared by adding 85 μl of sterile water, and 100 μl of two times Muller Hinton broth. Dissolved compounds (5 μl) from the master plate was added to the corresponding well in slave plates giving a final concentration of 50 μg/ml.

Plates were then transferred to a PC2 Laboratory for inoculation with selected bacterial strains. The strains are freshly grown and diluted in normal saline to 0.5 McFarland Standard (NCCLS Performance standard for Dilution Antimicrobial Susceptibility Testing M7-A4 January 1997). This solution was further diluted 1:10 in normal saline to form the bacterial inoculation culture. 10 μl was used to inoculate each well. Plates were covered and placed in a 35° C. incubator over night before A₆₂₀ was determined. Tetracycline was used as a standard antimicrobial compound.

### B. Results

Sixty three compounds from the CMS library were screened and two compounds were identified that significantly inhibited the growth of bacteria Specifically, compounds AOC-07877 and AOC-08944 both inhibited the growth of S. aureus and E. faecalis by more than 50% (see Table 22 below in which the values shown are percent growth inhibition). The former compound also exhibited a significant inhibitory activity on the interaction of δ and β. These results demonstrate the utility of the approaches described for the identification of chemical leads using peptide sequence data to search chemical diversity for mimetics of peptides.

TABLE 22
Effect on Bacterial Growth of Selected Chemical Compounds.

| | | Test | | | | | |
|---|---|---|---|---|---|---|---|
| | | Conc | | | | | |
| Number | Database | μg/ml | E. coli | K. pneumoniae | P. aeruginosa | S. aureus | E. faecalis |
| 07337 | molsci | 30 | −3 | −7.8 | 4.9 | −1.4 | 11.5 |
| 07262 | molsci | 32.5 | 3 | −8.1 | 2.1 | 6.6 | 42.9 |
| 07497 | molsci | 25 | 19.6 | 11.5 | 10.9 | 10.8 | 35.7 |
| 07336 | molsci | 35 | 2.1 | −2.9 | 4.6 | 6.7 | 42.9 |
| 07654 | molsci | 37.5 | 7.8 | 0.3 | 7.3 | −3.1 | 14.4 |
| 07263 | molsci | 30 | 7.6 | −4.5 | 5.9 | −19.2 | 31.5 |
| 07499 | molsci | 37.5 | 19.4 | 5.5 | −2 | 75.1 | 9.5 |
| 07338 | molsci | 35 | 18.1 | 12 | 3.5 | −6.2 | 17.6 |
| 08366 | molsci | 32.5 | 11.2 | 4.6 | −3.6 | 13.3 | −67.2 |
| 08271 | molsci | 25 | 16.9 | 5.5 | 1.1 | −15.3 | −31.4 |
| 07336 | molsci | 32.5 | 17.1 | 5.6 | 3.4 | −24.3 | −42.4 |
| 08462 | molsci | 25 | 15.4 | −70.5 | −4.8 | −39.2 | −585 |
| 08270 | molsci | 27.5 | 10.9 | −12.4 | −1.8 | −19.7 | −70.9 |
| 07244 | molsci | 27.5 | 3.5 | 7.9 | −0.7 | −23 | 31.7 |
| 07409 | molsci | 32.5 | 8.7 | 11.1 | 3.9 | −110.6 | 73.5 |
| 07875 | molsci | 32.5 | 25 | 20.2 | 5.9 | −24.4 | 36.9 |
| 07493 | molsci | 27.5 | −16.2 | −2.1 | 3 | −36.8 | 22.2 |
| 07245 | molsci | 27.5 | 4.8 | −7.8 | 0.3 | −23.7 | 18.8 |
| 07179 | molsci | 37.5 | −2 | −6.3 | 3.7 | −43.1 | 2.8 |
| 07494 | molsci | 32.5 | 6.6 | −17.1 | −1.8 | −77.5 | −4.6 |
| 07492 | molsci | 25 | −4.1 | 9.3 | 1.2 | −58.5 | −8 |
| 09623 | molsci | 35 | 5.5 | −1.7 | −0.8 | −27.1 | 32.5 |
| 09392 | molsci | 32.5 | 10.3 | −13 | 0.3 | −94.4 | 66.8 |
| 09102 | molsci | 25 | 1.9 | −21 | 0.9 | 29.9 | 15.8 |
| 09099 | molsci | 27.5 | 0.5 | −23.1 | −6 | 22.7 | −2.4 |
| 08179 | molsci | 30 | 3.9 | −35.8 | 1.1 | −13.3 | −122.7 |
| 09427 | molsci | 27.5 | 2.3 | 10.2 | −5.1 | −35.9 | 21.9 |
| 08180 | molsci | 37.5 | 7.8 | 37.5 | 3.9 | −21.3 | 154.6 |
| 07182 | molsci | 30 | 5.4 | 2.6 | −15.8 | −45.9 | −6 |
| 10041 | molsci | 35 | 8.4 | 17.7 | −6.1 | −51.5 | 11.9 |
| 07876 | molsci | 25 | 1.4 | −5.5 | −9.9 | 20.6 | 12.5 |
| 07495 | molsci | 25 | 4 | 8.9 | −0.3 | 10.9 | −2 |
| 07877 | molsci | 35 | 17.6 | 8.3 | 3.9 | 84.7 | 59.6 |
| 10040 | molsci | 35 | 11.8 | 7.4 | 4.5 | −10.6 | 8 |
| 07496 | molsci | 27.5 | 3.8 | 20.5 | 2.7 | 5.9 | 14.4 |
| 08944 | molsci | 25 | 10.5 | 9.5 | 13.5 | 101.8 | 87.1 |
| 10162 | molsci | 35 | 0.1 | 5.9 | −0.6 | 35 | 5.2 |
| 10114 | molsci | 32.5 | 6.7 | −9.4 | 2.5 | −43.4 | −71.4 |
| 10038 | molsci | 30 | 13.5 | −12.4 | 4.6 | −11.7 | −0.4 |
| 10115 | molsci | 25 | 24.3 | −17.1 | 15.2 | −23.4 | 3.4 |
| 06097 | molsci | 35 | 8.6 | −19.5 | −3.5 | −19.9 | 50.2 |
| 05155 | molsci | 27.5 | −4.2 | 8 | 7.9 | 22.1 | −33.2 |
| 06099 | molsci | 25 | 18.4 | 9.3 | 1.4 | 5.9 | −15.8 |
| 06242 | molsci | 32.5 | 7.9 | 5.2 | 12.3 | 11.9 | −4.3 |
| 05023 | molsci | 37.5 | −0.9 | 6.7 | 7.7 | 19.4 | −148.8 |
| 05099 | molsci | 25 | 5.6 | 1.2 | 4.6 | 26.8 | −79.7 |
| 05161 | molsci | 35 | 7.5 | 14.8 | 13.7 | 3 | −5.1 |
| 06572 | molsci | 25 | 6 | 5.9 | 9 | −27.8 | −67.9 |
| 05098 | molsci | 30 | −1.4 | 9.7 | 11.3 | 14.2 | −28.2 |
| 05154 | molsci | 25 | −3.2 | 8.5 | 0 | 5.9 | −20.4 |
| 04807 | molsci | 32.5 | −3.6 | 10.8 | −5.4 | 53.1 | 1.7 |
| 05638 | molsci | 25 | −4.6 | 9.3 | 5.5 | 17.6 | −39.5 |
| 05159 | molsci | 25 | −5.7 | 16.9 | 1.9 | 13.5 | −39.5 |
| 05001 | molsci | 37.5 | 1.4 | 8.5 | 11.8 | 47.1 | −11.6 |
| 05020 | molsci | 35 | 6.9 | 25.9 | −4.1 | 70.8 | 14 |
| 04852 | molsci | 27.5 | −3.5 | 8 | 3.2 | 38.9 | −19.9 |
| 06240 | molsci | 27.5 | −0.4 | 7.8 | −2 | 39.1 | −25.5 |
| 06243 | molsci | 25 | −1.9 | 8.7 | 4.5 | 28.7 | −23.4 |
| 05158 | molsci | 35 | −2.8 | 10 | 0.2 | −12.7 | −8.9 |
| 05646 | molsci | 25 | 4.2 | 13.7 | −3.5 | 22.1 | −17.2 |
| 06239 | molsci | 35 | 3.3 | −4.7 | −7.9 | 40.4 | −54.9 |
| 11230 | molsci | 32.5 | −2.7 | 1.3 | 9.9 | −4.7 | −14.1 |
| 04380 | molsci | 30 | −3.3 | −21 | 8.8 | −4.6 | 16 |

The structure of compound AOC-08944 follows:

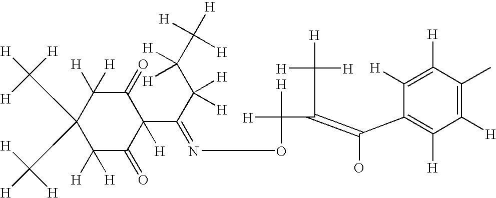

### EXAMPLE 15

In this example we illustrate the screening of representatives of a library of compounds for their ability to inhibit the binding of E. coli α to E. coli β.

### A. Methods

Compounds from the CMS library were dissolved in DMSO at 1 mg/ml in a 96 well tray format. A corresponding slave plate was prepared by adding 115 μl of BB37. Dissolved compounds (5 μl) from the master plate was added to the corresponding well in slave plates giving a final concentration of 41.7 μg/ml.

Compounds were assayed for inhibition of the binding of E. coli α to E. coli β as described in Example 13.

### B. Results

Sixty compounds from the CMS library were screened. One compound (AOL-06454: see structure below) was identified that significantly inhibited the binding of E. coli α to E. coli β.

TABLE 23
Inhibition of Binding of E. coli α To E. coli β of a
Chemical Compound

| Number | Database | Test Concentration | | % Inhibition |
|---|---|---|---|---|
| AOC-06454 | molsci | 41.7 υg/ml | 96 υM | 72.2, 75.3 |

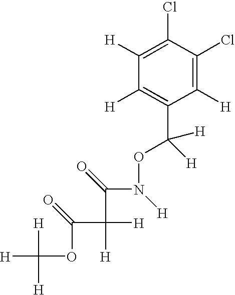

AOC-06454

The foregoing result demonstrates that the assays as described are suitable for the screening of large libraries of chemical compounds for compounds that inhibit the interaction of E. coli α and β.

### EXAMPLE 16

In this example, we describe the screening of additional peptides from E. coli β-binding proteins for their ability to inhibit the interaction of E. coli α and δ with E. coli β.

### A. Methods

Peptides were assayed for inhibition of the binding of E. coli α to E. coli β as described in Example 6 with the exception that buffer BB37 replaced buffer BB14 in the alpha:beta binding assay. As noted above, BB37 contains 10 mM MnCl₂ instead of 10 mM MgCl₂ used in BB14. Again, the change in buffer conditions was made to improve the reproducibility and sensitivity of the α:β binding assay.

### B. Results

A number of peptides from E. coli proteins containing putative β-binding sites were assayed for their ability to inhibit the interaction of E. coli α and δ with E. coli β. Some of the penta- and hexa-peptide motifs were flanked by the flanking sequences from E. coli α (peptides 110a-f, 112a and pep13) and some by their native flanking sequences (peptides 112c and d).

TABLE 24
Inhibition of Binding of E. coli α
to E. coli β by Peptides

| | | | | | |
|---|---|---|---|---|---|
| Source | Peptide | Seq. ID | | IC₅₀ α:β | IC₅₀ δ:β |
| Protein | Number | No. | Sequence | (μM) | (μM) |

| | | | | | |
|---|---|---|---|---|---|
| delta | 110a | 654 | IGQAMSL FGV | 27.0 | >100 |
| DinB1 | 110b | 655 | IGQ LVLGLGV | 9.3 | 6.8 |
| DnaA2 | 110c | 656 | IGQ LSLPLGV | 3.4 | 3.3 |
| UmuC2 | 110d | 657 | IGQ LNL FGV | 7.8 | 11.5 |
| MutS1 | 110e | 658 | IGQ MSL LGV | 9.7 | 7.0 |
| PolB2 | 110f | 659 | IGQ LGL FGV | 17.5 | 9.5 |
| DnaA2 | 112c | 660 | PAQ LSLPLYL | 1.2 | 2.1 |
| UmuC1 | 112d | 661 | EAQ LDL FDS | 1.0 | 3.6 |
| consensus 5-mer | 112f | 662 | Q LDL F | 2.8 | 6.1 |
| consensus 9-mer | pep13 | 663 | IGQ LSL FGV | 4.9 | 5.9 |

These results demonstrate that the pentapeptide motifs from E. coli UmuC1, UmuC2, MutS1 and PolB2 and the hexapeptide motifs from E. coli DinB1 and DnaA2 significantly inhibit the interaction of E. coli α:β and δ:β at levels similar to that observed for the consensus 9-mer (pep13). In addition, the consensus 5-mer (112f) exhibits a similar level of inhibition to the consensus 9-mer (pep13). Interestingly, the two most inhibitory peptides, DnaA2 and UmuC1, were flanked by their native flanking dipeptides suggesting the flanking amino acids may make contributions, albeit minor, to the binding ability of the peptides.

The comparable level of inhibitory activity of the pentapeptides and hexapeptides suggests that there are at least two, and from the bioinformatics analysis, possibly several more distinct families of β-binding peptides. The analysis of the consensus sequence for the hexapeptides suggests that the identity of the amino acid at position five, whilst small amino acids are favoured, is not critical and that the hydrophobic amino acid at position six is likely to be equivalent to the amino acid at position five in the pentapeptide motif.

It will be appreciated by one of skill in the art that many changes can be made to the aspects of the invention exemplified above without departing from the broad ambit and scope of the invention as defined in the following claims.

1. A method of identifying a modulator of the interaction between the β subunit of a eubacterial DNA polymerase III (β protein) and proteins that interact therewith by binding at a surface of said β protein defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:
X¹⁷⁰ is any one of V, I, A, T, S or E;
X¹⁷² is any one of T, S or I;
X¹⁷⁵ is any one of H, Y, F, K, I, Q or R;
X¹⁷⁷ is any one of L, M, I, F, V or A;
X²⁴¹ is any one of F, Y or L;
X²⁴² is any one of P, L or I;
X²⁴⁷ is any one of V, I, A, F, L or M;
X³⁴⁶ is any one of S, P, A, Y or K;
X³⁶⁰ is any one of I, L or V; and
X³⁶² is any one of M, L, V, S, T or R;
wherein said method comprises the steps of:
(a) forming a reaction mixture comprising:
(i) a ligand for eubacterial β protein that binds to at least part of said surface of β protein;
(ii) an interaction partner for said ligand; and
(iii) a test compound;
(b) incubating said reaction mixture under conditions which in the absence of said test compound allow interaction between said ligand and said interaction partner; and
(c) assessing the effect of said test compound on said interaction between said ligand and said interaction partner.

2. The method according to claim 1, wherein said ligand is selected from the group consisting of a protein, a peptide, an antibody, and a mimetic of said peptide.

3. The method according to claim 2, wherein said protein is selected from the group consisting of δ, DnaE1, DnaE2, PolC, PolB2, UmuC, DinB1, DinB2, DinB3, MutS1, RepA, Duf72 and DnaA2, and fragments thereof that bind to at least part of said surface of β protein.

4. The method according to claim 2, wherein said protein is selected from a fragment of δ, DnaE1, DnaE2, PolC, PolB2, UmuC, DinB1, DinB2, DinB3, MutS1, RepA, Duf72 and DnaA2 that binds to at least part of said surface of β protein, which fragment is fused to another protein.

5. The method according to claim 2, wherein said peptide selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P.

6. The method according to claim 2, wherein said ligand is a polypeptide or peptide that includes a sequence selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P.

7. The method according to claim 2, wherein said ligand is a polypeptide or peptide that includes any one of the motifs of Tables 1 to 13 and 15, or is a peptide comprising any one of the motifs of Tables 1 to 13 and 15.

8. The method according to claim 2, wherein said interaction partner is selected from the group consisting of eubacterial β protein, a fragment of eubacterial β protein that includes at least a functional portion of said surface of β protein, and a mimetic of said surface of β protein.

9. A method for the in vivo identification of a modulator of the interaction between the β subunit of a eubacterial DNA polymerase III (β protein) and proteins that interact therewith by binding at a surface of said β protein defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:
X¹⁷⁰ is any one of V, I, A, T, S or E;
X¹⁷² is any one of T, S or I;
X¹⁷⁵ is any one of H, Y, F, K, I, Q or R;
X¹⁷⁷ is any one of L, M, I, F, V or A;
X²⁴¹ is any one of F, Y or L;
X²⁴² is any one of P, L or I;
X²⁴⁷ is any one of V, I, A, F, L or M;
X³⁴⁶ is any one of S, P, A, Y or K;
X³⁶⁰ is any one of I, L or V; and
X³⁶² is any one of M, L, V, S, T or R;
wherein said method comprises the steps of:
(a) modifying a host to express or contain:
(i) a ligand for eubacterial β protein that binds to at least part of said surface of β protein; and
(ii) an interaction partner for said ligand;
(b) administering a test compound to said host and incubating the host under conditions which in the absence of said test compound allows interaction between said ligand and said interaction partner; and
(c) assessing the effect of said test compound on said interaction between said ligand and said interaction partner.

10. The method according to claim 9, wherein said host is selected from the group consisting of animal cells, plant cells, fungal cells, bacterial cells, bacteriophages and viruses.

11. The method according to claim 9, wherein said ligand is a protein selected from the group consisting of δ, DnaE1, DnaE2, PolC, PolB2, UmuC, DinB1, DinB2, DinB3, MutS1, RepA, Duf72 and DnaA2, and fragments thereof that bind to at least part of said surface of β protein.

12. The method according to claim 9, wherein said ligand is a peptide selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P.

13. The method according to claim 9, wherein said ligand is a polypeptide or peptide that includes a sequence selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P.

14. The method according to claim 9, wherein said ligand is a polypeptide or peptide that includes any one of the motifs of Tables 1 to 13 and 15, or is a peptide comprising any one of the motifs of Tables 1 to 13 and 15.

15. The method according to claim 9, wherein said interaction partner is selected from the group consisting of eubacterial β protein, and a fragment of eubacterial β protein that includes at least a functional portion of said surface of β protein.

16. A method of selecting a potential modulator of the interaction between the β subunit of a eubacterial DNA polymerase III (β protein) and proteins that interact therewith by binding at a surface of said β protein defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:
X¹⁷⁰ is any one of V, I, A, T, S or E;
X¹⁷² is any one of T, S or I;
X¹⁷⁵ is any one of H, Y, F, K, I, Q or R;
X¹⁷⁷ is any one of L, M, I, F, V or A;
X²⁴¹ is any one of F, Y or L;
X²⁴² is any one of P, L or I;
X²⁴⁷ is any one of V, I, A, F, L or M;
X³⁴⁶ is any one of S, P, A, Y or K;
X³⁶⁰ is any one of I, L or V; and
X³⁶² is any one of M, L, V, S, T or R;
wherein said method comprises the steps of:
(a) establishing a consensus sequence for peptides that bind to at least part of said surface of β protein;
(b) modelling the structure of at least a portion of said consensus sequence and searching compound databases for compounds having a similar structure; wherein said modelling is by:
(i) searching protein databases for occurrences of said consensus sequence or portion thereof, obtaining coordinates of residues of proteins comprising said consensus sequence or portion thereof, and superimposing said coordinates to produce a pharmacophore model; or
(ii) modelling or determining the structure of a peptide including said consensus sequence or a portion thereof when bound to β protein; and
(c) testing compounds identified in step (b) for their effect on said interaction.

17. The method according to claim 16, wherein said consensus sequence is selected from the sequence data of any one of Tables 1 to 13 and 15.

18. A method of reducing the effect of eubacterial infestation of a biological system, the method comprising delivering to a system infested with a eubacterial species a modulator of the interaction between the β subunit of eubacterial DNA polymerase III (β protein)and proteins that interact therewith by binding at a surface of said β protein defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:
X¹⁷⁰ is any one of V, I, A, T, S or E;
X¹⁷² is any one of T, S or I;
X¹⁷⁵ is any one of H, Y, F, K, I, Q or R;
X¹⁷⁷ is any one of L, M, I, F, V or A;
X²⁴¹ is any one of F, Y or L;
X²⁴² is any one of P, L or I;
X²⁴⁷ is any one of V, I, A, F, L or M;
X³⁴⁶ is any one of S, P, A, Y or K;
X³⁶⁰ is any one of I, L or V; and
X³⁶² is any one of M, L, V, S, T or R.

19. The method according to claim 18, wherein said modulator is a peptide selected from the group consisting of X¹X², X³X¹X², X³X¹X²X⁴, QX⁵X³X¹X², and QX⁵xX⁶X³X⁶, wherein: X is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P.

20. The method according to claim 18, wherein said modulator is a mimetic of any one of the peptides defined in claim 19.

21. The method according to claim 18, wherein said modulator is an inhibitor of the interaction between eubacterial β protein and proteins that interact therewith.

22. A method of selecting a potential modulator of the interaction between the β subunit of a eubacterial DNA polymerase III (β protein) and proteins that interact therewith by binding at a surface of said β protein defined by the residues X¹⁷⁰, X¹⁷², X¹⁷⁵, X¹⁷⁷, X²⁴¹, X²⁴², X²⁴⁷, X³⁴⁶, X³⁶⁰ and X³⁶², wherein the superscript numbers designate the position of residues in Escherichia coli β protein, or the equivalent residues in homologues from other species of eubacteria, and wherein:
X¹⁷⁰ is any one of V, I, A, T, S or E;
X¹⁷² is any one of T, S or I;
X¹⁷⁵ is any one of H, Y, F, K, I, Q or R;
X¹⁷⁷ is any one of L, M, I, F, V or A;
X²⁴¹ is any one of F, Y or L;
X⁴² is any one of P, L or I;
X²⁴⁷ is any one of V, I, A, F, L or M;
X³⁴⁶ is any one of S, P, A, Y or K;
X³⁶⁰ is any one of I, L or V; and
X³⁶² is any one of M, L, V, S, T or R;
wherein said method comprises the steps of:
(a) designing a mimetic of a peptide selected from the group consisting of X¹X², X³X¹X², X³X¹X²X⁴, QX⁵X³X¹X², and QX⁵xX⁶X³X⁶, wherein: x is any amino acid residue; X¹ is L, M, I, or F; X² is L, I, V, C, F, Y, W, P, D, A or G; X³ is A, G, T, N, D, S, or P; X⁴ is A or G; X⁵ is L; and, X⁶ is L, I, V, C, F, Y, W or P;
(b) testing said mimetic for its effect on said interaction.

23. The method according to claim 22, wherein said peptide is selected from the group consisting of: QLSLF (Seq. ID No. 622); QLSMF (Seq. ID No. 623); QLDMF (Seq. ID No. 624); QLDLF (Seq. ID No. 625); HLSLF (Seq. ID No. 626); HLSMF (Seq. ID No. 627); HLDMF (Seq. ID No. 628); HLDLF (Seq. ID No. 629); X³LFX⁴; SLF; SMF; DLF; DMF; LF; and MF.

24. The method according to claim 22, wherein said peptide is any one of the motifs of Tables 1 to 13 and 15.
